(19) United States
(12) Patent Application Publication
(10) Pub. No.: US 2022/0090127 A1
(43) Pub. Date: Mar. 24, 2022

(54) VIRAL VECTOR COMBINING GENE THERAPY AND GENOME EDITING APPROACHES FOR GENE THERAPY OF GENETIC DISORDERS

(71) Applicants: INSTITUT NATIONAL DE LA SANTE ET DE LA RECHERCHE MEDICALE (INSERM, Paris (FR); UNIVERSITE PARIS DESCARTES, Paris (FR); ASSISTANCE PUBLIQUE-HOPITAUX DE PARIS, Paris (FR); MAGINE - INSTITUT DES MALADIES GENETIQUES NECKER ENFANTS MALADES, Paris (FR)

(72) Inventors: Annarita MICCIO, Paris (FR); Vasco MENEGHINI, Bretigny-Sur-Orge (FR)

(21) Appl. No.: 16/618,251

(22) PCT Filed: Jun. 1, 2018
(86) PCT No.: PCT/EP2018/064532

§ 371 Date: Nov. 29, 2019

(30) Foreign Application Priority Data
Jun. 2, 2017 (EP) 17305649.0

(51) Int. Cl. C12N 15/86; C12N 15/11; C12N 15/90; C12N 9/22; A61K 35/28; A61P 7/00; A61K 31/713; A61K 38/46

(52) U.S. Cl. CPC C12N 15/86; C12N 15/11; C12N 15/907; C12N 9/22; A61K 35/28; C12N 2740/15043; A61K 31/713; A61K 38/465; C12N 2750/14343; C12N 2310/20; C12N 2800/80; A61P 7/00

(57) ABSTRACT

This invention relates to recombinant viral vectors, preferably retroviral (RV), lentiviral (LV) or adeno-associated viral (AAV) vectors, compositions thereof, the use of the recombinant viral vectors or the compositions thereof, kits of parts comprising said recombinant viral vectors or compositions thereof and a catalytically active Cas9 or Cpf1 protein, methods for modifying the genome of a cell, and the cells obtainable by such methods.

### BACKGROUND OF THE INVENTION

Many human disorders have a genetic component and are called “genetic disorders” (or “genetic diseases”). A genetic disorder is caused by one or more abnormalities in the genome, said abnormalities are generally gene mutations, and said mutations generally alter the function of a protein.

Genetic disorders may be hereditary and passed on from family members or non-heritable and acquired during a person's lifetime. Acquired genetic disorders refer to conditions caused by acquired abnormalities in the genome. These conditions only become heritable if the abnormalities occur in the germ line.

There are a number of different types of genetic disorders:
Single gene disorders (also called Mendelian or monogenic inheritance): this type of inherited disorder is caused by changes or mutations that occur in the DNA sequence of a single gene. There are more than 6,000 known single-gene disorders, which occur in about 1 out of every 200 births. Some examples are sickle cell disorder, immune-deficiencies, Marfan syndrome, Huntington's disease, and hereditary hemochromatosis type 4, congenital hyperinsulinism, hereditary spherocytosis, neutropenia-1, Li-Fraumeni syndrome. Single-gene disorders are inherited in recognizable patterns: autosomal dominant, autosomal recessive, and X-linked.
Multifactorial inheritance (also called complex or polygenic inheritance): this type of inheritance is caused by a combination of environmental factors and mutations in multiple genes. Some common chronic diseases are multifactorial disorders. Examples include heart disease, high blood pressure, Alzheimer disease, arthritis, diabetes, cancer, and obesity.
Chromosome abnormalities: chromosomes, distinct structures made up of DNA and protein, are located in the nucleus of each cell. Because chromosomes are the carriers of the genetic material, abnormalities in chromosome number or structure can result in disease. For example, Down's syndrome or trisomy 21 is a common disorder that occurs when a person has three copies of chromosome 21. There are many other chromosome abnormalities including Turner syndrome, Klinefelter syndrome, the cat cry syndrome.
Mitochondrial inheritance: this type of genetic disorder is caused by mutations in the nonchromosomal DNA of mitochondria. Mitochondria are small round or rod-like organelles that are involved in cellular respiration and found in the cytoplasm of plant and animal cells. Each mitochondrion may contain 5 to 10 circular pieces of DNA. Examples of mitochondrial disease include an eye disease called Leber's hereditary optic atrophy; a type of epilepsy called MERRF which stands for Myoclonus Epilepsy with Ragged Red Fibers; and a form of dementia called MELAS for Mitochondrial Encephalopathy, Lactic Acidosis and Stroke-like episodes.

Most genetic disorders are single gene disorders. A single gene disorder can be either dominant (Autosomal dominant) or recessive (Autosomal recessive).

If autosomal dominant, only one mutated copy of the gene will be necessary for a person to be affected by an autosomal dominant disorder. In general, each affected person usually has one affected parent. The chance a child will inherit the mutated gene is therefore 50%. Autosomal dominant conditions sometimes have reduced penetrance, which means although only one mutated copy is needed, not all individuals who inherit that mutation go on to develop the disease. Examples of this type of disorder are Huntington's disease, neurofibromatosis type 1, neurofibromatosis type 2, Marfan syndrome, hereditary nonpolyposis colorectal cancer, hereditary multiple exostoses (a highly penetrant autosomal dominant disorder), Tuberous sclerosis, Von Willebrand disease, and acute intermittent porphyria.

If autosomal recessive, two copies of the gene must be mutated for a person to be affected by an autosomal recessive disorder. An affected person usually has unaffected parents who each carry a single copy of the mutated gene. Two unaffected people who each carry one copy of the mutated gene have therefore a 25% risk with each pregnancy of having a child affected by the disorder. Example of this type of disorders is sickle-cell disease.

The treatments of genetic disorders have led to the development of new therapies, such as gene therapy. Gene therapy refers to a form of treatment where a functional gene (or a nucleotide sequence encoding a protein that has a therapeutic effect) is introduced into a patient's cells. This should alleviate the defect caused by an altered gene or slow the progression of disease. Gene therapy is defined by the precision of the procedure and the intention of direct therapeutic effects. Gene therapy is therefore a way to fix a genetic problem at its source.

Two main approaches have been considered for gene therapy: replacing defective genes or disrupting defective genes. In these approaches therapeutic DNA must enter the cell, replace/disrupt a gene and thus express/disrupt a protein. Thus, a major obstacle has been the delivery of genes to the appropriate cell, tissue, and organ affected by the disorder.

Multiple delivery techniques have been explored. The initial approach has been to incorporate the therapeutic DNA into an engineered virus to deliver the therapeutic DNA into the patient's cells. Naked DNA approaches for transfection into the patient's cells have also been explored in order to deliver the therapeutic DNA into the target cells.

More recently, new approaches have led to more direct DNA editing, using techniques such as zinc finger nucleases, TALENs or CRISPR. The aim of these approaches is to express nucleases that knock-out, correct or edit specific genes in the genome. These approaches involve: (i) ex vivo approaches based on removing cells from patients, editing a gene and returning the transformed cells to patients; (ii) in vivo approaches based on the delivery of nucleases through viral vectors or nanoparticles into target tissues.

Furthermore, the above mentioned approaches are only designed to either incorporate a therapeutic DNA into the patient's cells or to edit an altered gene in the patient's cells. These approaches are not designed to both incorporate a therapeutic DNA into the patient's cells and to knock-out an altered gene in the patient's cells. This double function may be particularly useful for treating genetic disorders, for example autosomal dominant genetic disorders or recessive genetic disorders, in which the expression of the endogenous mutated protein compromise the beneficial effects induced by the expression of the exogenous corrected protein.

Thus, there is a need to find new easy to practice approaches both for incorporating a therapeutic DNA into a patient's cell and to knock-out an altered gene in said patient's cells.

### SUMMARY OF THE INVENTION

The inventors propose here new recombinant viral vector and process for gene therapy that is particularly efficient and easy to practice for both incorporate a therapeutic DNA into a patient's cell (i.e. “gene addition”) and to knock-out an altered gene in said patient's cell (i.e. “gene editing”).

The invention relates to a recombinant viral vector comprising in its genome:
(i) a nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder; and.
(ii) a nucleotide sequence encoding a protein that has a therapeutic effect in said genetic disorder.

The invention also relates to a composition comprising a recombinant viral vector according to the invention or a plurality of recombinant viral vectors according to the invention.

The invention also relates to a kit of parts comprising:
a recombinant viral vector of the invention or a composition of the invention; and
a catalytically active Cas9 or Cpf1 protein or a nucleotide sequence encoding a catalytically active Cas9 or Cpf1 protein.

The invention also relates to the use of a recombinant viral vector of the invention or a composition of the invention for introducing into a cell (i) nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder and (ii) a nucleotide sequence encoding a protein that has a therapeutic effect in said genetic disorder.

The invention also relates to a method for modifying the genome of a cell in vitro, ex vivo or in vivo comprising the steps of:
a) contacting a cell with a recombinant viral vector of the invention or a composition of the invention to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

The invention also relates to a method for preparing a genetically modified cell in vitro, ex vivo or in vivo, comprising the steps of:
a) contacting a cell with a recombinant viral vector of the invention or a composition of the invention to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

The invention also relates to a cell obtainable by the methods of the invention.

### DETAILED DESCRIPTION OF THE INVENTION

### Recombinant Vector

The invention relates to a recombinant viral vector comprising in its genome:
(i) a nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder.
(ii) a nucleotide sequence encoding a protein that has a therapeutic effect in said genetic disorder.

The recombinant viral vector according to the invention, when transduced into a cell (transduced cell), provides expression of the protein that has a therapeutic effect and the gRNA into said transduced cell and/or into a differentiate progeny of the transduced cell. Viruses are commonly used as a vector or delivery system for the transfer of nucleotide sequences to a cell. The transfer can occur in vitro, ex vivo or in vivo. When used in this fashion, the viruses are typically called “viral vectors”. In a preferred embodiment of the present invention, the viral vector is a retroviral (RV) vector or an adeno-associated viral (AAV) vector. The retroviral vectors according to the invention are a virus particles that contain a retrovirus-derived viral genome, lack the self-renewal ability, and have the ability to introduce a nucleotide sequence into a cell. The AAV vectors according to the invention are virus particles that contain a AAV-derived genome, lack the self-renewal ability, and have the ability to introduce a nucleotide sequence into a cell.

“Recombinant” is used consistently with its usage in the art to refer to a nucleotide sequence that comprises portions that do not naturally occur together as part of a single sequence or that have been rearranged relative to a naturally occurring sequence. A recombinant nucleotide sequence (or transgene) is created by a process that involves the human intervention and/or is generated from a nucleic acid that was created by human intervention (e.g., by one or more cycles of replication, amplification, transcription, etc.). A recombinant virus is one that comprises a recombinant nucleotide sequence. A recombinant cell is one that comprises in its genome a recombinant nucleotide sequence. Thus, a “recombinant viral vector” (e.g. a “recombinant retroviral vector” or a “recombinant AAV vector”) according to the invention refers to a viral vector comprising in its genome a recombinant nucleotide sequence (or transgene).

The recombinant viral “genome”, as used herein, accordingly contains, apart from the so-called recombinant nucleotide sequences placed under control of proper regulatory sequences for its expression, the sequences of the original virus which are non-coding regions of said genome, and are necessary to provide recognition signals for DNA or RNA synthesis and processing (mini-viral genome). For example, for recombinant lentiviral vectors, these sequences are cis-acting sequences necessary for packaging, reverse transcription and transcription and furthermore for the particular purpose of the invention, they contain a functional sequence favoring nuclear import in cells and accordingly transgenes transfer efficiency in said cells, which element is described as a DNA Flap element.

The recombinant viral vector can be based on any suitable virus which is able to deliver genetic information to eukaryotic cells, in particular to mammalian cells, in particular to a human cell. In some embodiments, for in-vivo approaches, the cells are stem cells, progenitor cells or differentiated cells. In some embodiments, for ex-vivo and in vitro approaches, the cells are a stem cell, e.g. a human stem cell, progenitor cells or differentiated cells, e.g. T lymphocytes.

In some embodiment, the viral vector of the invention is a retroviral vector or an adeno-associated vector.

For example, the retroviral vector may be an alpha-retroviral vector, a gamma-retroviral vector, a lentiviral vector or a spuma-retroviral vector, preferably a lentiviral vector. Such vectors have been used extensively in gene therapy treatments and other gene delivery applications. In a preferred embodiment, the retroviral vector is a lentiviral vector. In some embodiment, the lentiviral vector is a “lentiviral integrative vector”.

The terms “lentiviral vector”, as used herein, refers to viral vector derived from complex retroviruses such as the human immunodeficiency virus (HIV). In the present invention, lentiviral vectors derived from any strain and subtype can be used. The lentiviral vector may be based on a human or primate lentivirus such as HIV or a non-non-human lentivirus such as Feline immunodeficiency virus, simian immunodeficiency virus and equine infectious anemia virus (EIAV). In a preferred embodiment, the lentiviral vector is a HIV-based vector and especially a HIV-1-based vector.

By an “AAV vector” is meant a viral vector derived from an adeno-associated virus serotype, including without limitation, AAV-1, AAV-2, AAV-3, AAV-4, AAV-5, AAV6, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e.g, by the insertion, deletion or substitution of nucleotides, so long as the sequences provide for functional rescue, replication and packaging. AAV vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is bounded (5′ and Y) with functional AAV ITR sequences. By “adeno-associated virus inverted terminal repeats” or “AAVITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e. g., Kotin, 1994; Berns, K I “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e. g., by the insertion, deletion or substitution of nucleotides. Additionally, the AAV ITR may be derived from any of several AAV serotypes, including without limitation, AAV-1, AAV-2, AAV-3, AAV-4, AAV-5, AAV6, etc. Furthermore, 5′ and 3′ITRs which flank a selected nucleotide sequence in an AAV vector need not necessarily be identical or derived from the same AAV serotype or isolate, so long as they function as intended, i. e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV6, etc. Furthermore, 5′ and 3′ITRs which flank a selected nucleotide sequence in an AAV vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell. The selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the phophoglycerate kinase (PKG) promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), rous sarcoma virus (RSV) promoter, synthetic promoters, hybrid promoters, and the like. In addition, sequences derived from non-viral genes, such as human beta AS3 globin gene or HTT, will also find use herein. Such promoter sequences are commercially available from, e. g., Stratagene (San Diego, Calif.). For purposes of the present invention, both heterologous promoters and other control elements, such as CNS-specific and inducible promoters, enhancers and the like, will be of particular use.

In the recombinant viral vectors of the present invention, the recombinant nucleotide sequences encode a protein that has a therapeutic effect and a gRNA that comprises a spacer (i.e. a gRNA spacer) adapted to bind to a target nucleotide sequence. The terms “protein that has a therapeutic effect” means a protein that provides an effect which is judged to be desirable and beneficial to a patient, in particular a patient with a genetic disorder. Examples of a protein that has a therapeutic effect in the present invention may be a protein that has become dysfunctional due to a genetic disease. Thus, in one embodiment, the term “protein that has a therapeutic effect” refers to a protein that does not produce a genetic disorder, and which is effective to provide therapeutic benefits to a patient, in particular a patient with a genetic disorder. The protein that has a therapeutic effect may be a wild-type (WT) protein appropriate for a patient with a genetic disorder to be treated, or it may be a mutant form of the WT protein (i.e. a variant of the WT protein) appropriate for a patient to be treated. The protein that has a therapeutic effect may also be a protein with similar or improved features compared to the “wild-type protein appropriate for a patient”.

In a specific embodiment, the intended patient is a mammalian being, preferably a human being, regardless of age and gender. In particular, the patient has a genetic disorder, said genetic disorder is disclosed below.

In some embodiment, the protein that has a therapeutic effect is an eukaryotic protein, preferably a mammalian protein, preferably a human protein.

According to the invention, the target gene is involved in a genetic disorder. In other words, the target gene is involved in a genetic disorder when the corresponding protein (target protein) is expressed in a subject. In one embodiment, the target gene causes a genetic disorder, for example the protein that has a therapeutic effect is involved in a genetic disorder when said protein is altered in a patient. The target protein is therefore an altered version of the protein that has a therapeutic effect. In another embodiment, the target gene is a genetic modifier of the genetic disorder but is not the gene that causes the genetic disorder.

The terms “protein is altered” or “altered protein” means a change (increase or decrease) in the expression levels or activity of the protein, or a change in the structural conformation or interaction properties of the protein. An altered protein may cause a genetic disorder.

In some embodiment, the genetic disorder is selected from the group consisting of:

| Disease | Inheritance |
|---|---|
| Achondroplasia | autosomal dominant |
| acute intermittent porphyria | autosomal dominant |
| Alpha-1 antitrypsin deficiency | autosomal codominant |
| Alport syndrome | autosomal dominant |
| Amyotrophic lateral sclerosis | autosomal dominant |
| autoimmune lymphoproliferative syndrome | autosomal dominant |
| type V | |
| autosomal dominant anhidrotic ectodermal | autosomal dominant |
| dysplasia with T-cell immunodeficiency | |
| Autosomal dominant congenital stationary | autosomal dominant |
| night blindness | |
| Autosomal dominant hyper-IgE syndrome | autosomal dominant |
| Charcot-Marie-Tooth | autosomal dominant |
| Chronic Mucocutaneous Candidiasis | autosomal dominant |
| Common variable immune deficiency 10 | autosomal dominant |
| Common variable immune deficiency 12 | autosomal dominant |
| Common variable immune deficiency 13 | autosomal dominant |
| Common variable immune deficiency 2 | autosomal dominant |
| Congenital hyperinsulinism | autosomal dominant |
| Cowden syndrome | autosomal dominant |
| Denys-Drash syndrome | autosomal dominant |
| Diffuse-type gastric carcinoma | autosomal dominant |
| dyskeratosis congenita-1 | autosomal dominant |
| Dystonia 6 | autosomal dominant |
| dystrophic epidermolysis bullosa pruriginosa | autosomal dominant |
| Early-onset primary dystonia | autosomal dominant |
| Ehlers-Danlos syndrome type IV | autosomal dominant |
| Ehlers-Danlos syndrome type VII | autosomal dominant |
| epidermolysis bullosa dystrophica | autosomal dominant |
| epidermolysis bullosa simplex | autosomal dominant |
| Familial adenomatous polyposis | autosomal dominant |
| familial breast-ovarian cancer-1 | autosomal dominant |
| familial retinoblastoma | autosomal dominant |
| Fragile X syndrome | X-linked dominant |
| Hereditary hemochromatosis type 4 | autosomal dominant |
| Hereditary hemorrhagic telangiectasia | autosomal dominant |
| Hereditary leiomyomatosis and renal cell cancer | autosomal dominant |
| Hereditary prostate cancer | autosomal dominant |
| hereditary spastic paraplegia type 31 | autosomal dominant |
| hereditary spastic paraplegia type 3A | autosomal dominant |
| hereditary spastic paraplegia type 4 | autosomal dominant |
| hereditary spastic paraplegia type 8 | autosomal dominant |
| Hereditary spherocytosis | autosomal dominant |
| Huntington disease | autosomal dominant |
| hyper-IgE recurrent infection syndrome | autosomal dominant |
| Hypercholesterolemia | autosomal dominant |
| Hyperkalemic periodic paralysis | autosomal dominant |
| Hypokalemic periodic paralysis | autosomal dominant |
| immunodeficiency-13 | autosomal dominant |
| immunodeficiency-14 | autosomal dominant |
| immunodeficiency-21 | autosomal dominant |
| immunodeficiency-27B | autosomal dominant |
| immunodeficiency-31A | autosomal dominant |
| immunodeficiency-31C | autosomal dominant |
| immunodeficiency-32A | autosomal dominant |
| immunodeficiency-36 | autosomal dominant |
| immunodeficiency-45 | autosomal dominant |
| immunodeficiency-49 | autosomal dominant |
| Immunoglobulin A (IgA) deficiency-2 | autosomal dominant |
| Incontinentia pigmenti | X-linked dominant |
| Infantile-onset spinocerebellar ataxia | autosomal dominant |
| Li-Fraumeni syndrome | autosomal dominant |
| Lynch syndrome | autosomal dominant |
| Marfan syndrome | autosomal dominant |
| maturity-onset diabetes of the young | autosomal dominant |
| mental retardation-43 | autosomal dominant |
| Multiple endocrine neoplasia | autosomal dominant |
| Multiple exostoses type I | autosomal dominant |
| Multiple exostoses type II | autosomal dominant |
| Myoclonus-dystonia | autosomal dominant |
| Myotonic dystrophy | autosomal dominant |
| Neurofibromatosis type 1 | autosomal dominant |
| Neurofibromatosis type 2 | autosomal dominant |
| neutropenia-1 | autosomal dominant |
| nevoid basal cell carcinoma syndrome | autosomal dominant |
| Osteogenesis imperfecta | autosomal dominant |
| Peutz-Jeghers syndrome | autosomal dominant |
| Polycystic kidney disease | autosomal dominant |
| Rapid-onset dystonia parkinsonism | autosomal dominant |
| Retinitis pigmentosa | autosomal dominant |
| Sickle cell disorder | autosomal recessive |
| Spinal muscular atrophy, lower extremity, | autosomal dominant |
| dominant (SMA-LED) and adult-onset | |
| form of spinal muscular atrophy | |
| Spinocerebellar ataxia type 1 | autosomal dominant |
| Spinocerebellar ataxia type 2 | autosomal dominant |
| Spinocerebellar ataxia type 3 | autosomal dominant |
| Spinocerebellar ataxia type 36 | autosomal dominant |
| Spinocerebellar ataxia type 6 | autosomal dominant |
| Tuberous sclerosis complex | autosomal dominant |
| Von Hippel-Lindau syndrome | autosomal dominant |
| Von Willebrand disease type I and II | autosomal dominant |

In some embodiment, the protein that has a therapeutic effect is:

| Disease | Protein that has a therapeutic effect |
|---|---|
| Achondroplasia | FGFR3 |
| acute intermittent porphyria | PBGD |
| Alpha-1 antitrypsin deficiency | SERPINA1 |
| Alport syndrome | COL4A3 or COL4A4 |
| Amyotrophic lateral sclerosis | C9orf72, SOD1, TARDBP, FUS, ALS2, ANG, ATXN2, |
| | CHCHD10, CHMP2B, DCTN1, ERBB4, FIG4, HNRNPA1, |
| | MATR3, NEFH, OPTN, PFN1, PRPH, SETX, SIGMAR1, |
| | SMN1, SPG11, SQSTM1, TBK1, TRPM7, TUBA4A, |
| | UBQLN2, VAPB or VCP |
| autoimmune lymphoproliferative | CTLA4 |
| syndrome type V | |
| autosomal dominant anhidrotic | NFKBIA |
| ectodermal dysplasia with T-cell | |
| immunodeficiency | |
| Autosomal dominant congenital | RHO, GNAT1 or PDE6B |
| stationary night blindness | |
| Autosomal dominant hyper-IgE | STAT3 |
| syndrome | |
| Charcot-Marie-Tooth | PMP22, MPZ, LITAF, EGR2, NEFL, MFN2, KIF1B, |
| | RAB7A, LMNA, TRPV4, BSCL2, GARS, HSPB1, MPZ, |
| | GDAP1, HSPB8, DNM2, YARS, GJB1 or PRPS1 |
| Chronic Mucocutaneous | STAT1 |
| Candidiasis | |
| Common variable immune | NFKB2 |
| deficiency 10 | |
| Common variable immune | NFKB1 |
| deficiency 12 | |
| Common variable immune | IKZF1 |
| deficiency 13 | |
| Common variable immune | TNFRSF13B |
| deficiency 2 | |
| Congenital hyperinsulinism | ABCC8, KCNJ11, GLUD1, HADH, HNF1A, HNF4A, |
| | SLC16A1 or UCP2 |
| Cowden syndrome | PTEN, SDHB, SDHD, or KLLN |
| Denys-Drash syndrome | WT1 |
| Diffuse-type gastric carcinoma | RHOA |
| dyskeratosis congenita-1 | TERC |
| Dystonia 6 | THAP1 |
| dystrophic epidermolysis bullosa | COL7A1 |
| pruriginosa | |
| Early-onset primary dystonia | TOR1A |
| Ehlers-Danlos syndrome type IV | COL3A1 |
| Ehlers-Danlos syndrome type VII | COL1A1 or COL1A2 |
| epidermolysis bullosa dystrophica | COL7A1 |
| epidermolysis bullosa simplex | KRT5, KRT15, PLEC1 or ITGB4 |
| Familial adenomatous polyposis | APC |
| familial breast-ovarian cancer-1 | BRCA1 |
| familial retinoblastoma | RB1 |
| Fragile X syndrome | FMR1 |
| Hereditary hemochromatosis | SLC40A1 |
| type 4 | |
| Hereditary hemorrhagic | ACVRL1, ENG or SMAD4 |
| telangiectasia | |
| Hereditary leiomyomatosis and | FH |
| renal cell cancer | |
| Hereditary prostate cancer | BRCA1, BRCA2 or HOXB13 |
| hereditary spastic paraplegia | REEP1 |
| type 31 | |
| hereditary spastic paraplegia | ATL1 |
| type 3A | |
| hereditary spastic paraplegia | SPAST |
| type 4 | |
| hereditary spastic paraplegia | WASHC5 |
| type 8 | |
| Hereditary spherocytosis | ANK1, EPB42, SLC4A1, SPTa1 or SPTB |
| Huntington disease | HTT |
| hyper-IgE recurrent infection | STAT3 |
| syndrome | |
| Hypercholesterolemia | LDLR, APOB or PCSK9 |
| Hyperkalemic periodic paralysis | SCN4A |
| Hypokalemic periodic paralysis | CACNA1S or SCN4A |
| immunodeficiency-13 | UNC119 |
| immunodeficiency-14 | PIK3CD |
| immunodeficiency-21 | GATA2 |
| immunodeficiency-27B | IFNGR1 |
| immunodeficiency-31A | STAT1 |
| immunodeficiency-31C | STAT1 |
| immunodeficiency-32A | IRF8 |
| immunodeficiency-36 | PIK3R1 |
| immunodeficiency-45 | IFNAR2 |
| immunodeficiency-49 | BCL11B |
| Immunoglobulin A (IgA) | TNFRSF13B |
| deficiency-2 | |
| Incontinentia pigmenti | IKBKG |
| Infantile-onset spinocerebellar | TWNK |
| ataxia | |
| Li-Fraumeni syndrome | p53 or CHEK2 |
| Lynch syndrome | MLH1, MSH2, MSH6, PMS2 or EPCAM |
| Marfan syndrome | FBN1 |
| maturity-onset diabetes of the | HNF4A, GCK, HNF1A, PDX1, TCF2, NEUROD1, KLF11, |
| young | CEL, PAX4, INS, BLK, KCNJ11 or APPL1 |
| mental retardation-43 | HIVEP2 |
| Multiple endocrine neoplasia | MEN1, RET or CDKN1B |
| Multiple exostoses type I | EXT1 |
| Multiple exostoses type II | EXT2 |
| Myoclonus-dystonia | SGCE |
| Myotonic dystrophy | DMPK or CNBP |
| Neurofibromatosis type 1 | NF1 |
| Neurofibromatosis type 2 | NF2 |
| neutropenia-1 | ELANE |
| nevoid basal cell carcinoma | PTCH1 |
| syndrome | |
| Osteogenesis imperfecta | COL1A1, COL1A2, CRTAP or P3H1 |
| Peutz-Jeghers syndrome | STK11 |
| Polycystic kidney disease | PKD1 or PKD2 |
| Rapid-onset dystonia parkinsonism | ATP1A3 |
| Retinitis pigmentosa | RHO, RP1, PRPH2RP9, IMPDH1, PRPF31, PRPF8, CA4, |
| | PRPF3, ABCA4, NRL, FSCN2, TOPORS, SNRNP200, |
| | SEMA4A, NR2E3, KLHL7, RGR, GUCA1B, BEST1, PRPF6 |
| | or PRPF4 |
| Sickle cell disorder | beta-globin, gamma-globin, delta-globin, beta-globin |
| | harboring one Thr87Gln mutation, beta-globin |
| | harboring three mutations Gly16Asp, Glu22Ala and |
| | Thr87Gln, gamma-globin harboring two mutations |
| | Gly16Asp and Glu22Ala or delta-globin harboring one |
| | mutation Gly16Asp |
| Spinal muscular atrophy, lower | VAPB |
| extremity, dominant (SMA-LED) | |
| and adult-onset form of spinal | |
| muscular atrophy | |
| Spinocerebellar ataxia type 1 | ATXN1 |
| Spinocerebellar ataxia type 2 | ATXN2 |
| Spinocerebellar ataxia type 3 | ATXN3 |
| Spinocerebellar ataxia type 36 | NOP56 |
| Spinocerebellar ataxia type 6 | CACNA1A |
| Tuberous sclerosis complex | SC1 or TSC2 |
| Von Hippel-Lindau syndrome | VHL |
| Von Willebrand disease type I and | VWF |
| II | |

The official symbols of beta-like globin genes are: HBB (beta-globin gene), HBD (delta-globin gene), HBG1 and HBG2 (gamma-globin genes), HBA1 and HBA2 (alpha-globin genes). The Greek symbols (e.g. α, β, γ and δ) and the corresponding denomination (e.g. alpha, beta, gamma, and delta) are used independently in the present description. Furthermore, the beta-like globin genes/mRNA/proteins are independently used in italic or not in the present description (e.g. HBB gene or HBB gene; HBB mRNA and HBB mRNA and HBB protein or HBB protein).

The term “gamma-globin target gene” means HBG1, HBG2 or both HBG1 and HGB2.

According to the invention, the gRNA comprises a spacer (said spacer is also called “CRISPR spacer” or “gRNA spacer” in the present description) adapted to bind to a target nucleotide sequence. The terms “target nucleotide sequence” means any endogenous nucleic acid sequence of the genome of a cell, such as, for example a gene or a non-coding sequence within or adjacent to a gene, in which it is desirable modify by targeted non-homologous end-joining (NHEJ) or MMEJ (Microhomology-mediated end-joining), in particular to disrupt (e.g. to knock-out) the expression and/or the function of said gene (also called “target gene”). The target nucleotide sequence can be present in a chromosome. In some embodiments, the target nucleotide sequence is within the coding sequence of the target gene or within a transcribed non-coding sequence of the target gene such as, for example, leader sequences, trailer sequence or introns. According to the invention, the target gene is known to be involved in a sickle cell disease (SCD) when said target gene is expressed in a patient.

Generally, the nucleotide sequence encoding the gRNA is designed to encode a gRNA that may disrupt the expression and/or the function of a target gene through the insertion of frameshift mutations in its coding sequence. Thus, the nucleotide sequence encoding the gRNA is designed to encode a gRNA that may disrupt the function and/or the expression of a target protein. This disruption takes place when said gRNA forms a complex with Cas9 or Cpf1 in the transduced cell through the CRISPR/Cas9 system or CRISPR/Cpf1 system respectively (see below).

Thus, according to the invention, the recombinant viral vector provides expression of the protein that has a therapeutic effect and the gRNA into a cell transduced by said recombinant viral vector (also called “transduced cell”). The transduced cell therefore expresses a gRNA that may disrupt the function and/or the expression of a target protein in the transduced cell by forming a complex with Cas9 or Cpf1. According to the invention, a non-transcribed sequence, either upstream or downstream of a target gene may be a region regulating the expression of a target gene, for example a promoter or an enhancer. In some embodiment, the target gene is involved in a genetic disorder when said target gene is expressed in a patient.

The terms “disrupt the function of a target protein” or “target protein is disrupted” or “disrupted target protein” means a decrease in the expression levels and/or activity of the target protein. Thus, the terms “disrupt the function of a target gene” or “target gene is disrupted” or “disrupted target gene” means a decrease in the expression level and/or function of the target gene.

The term “to disrupt” comprises “to knock out”. In a specific embodiment, the gRNA knocks-out the expression and/or the function of the target gene and therefore the gRNA knocks out the expression and/or the activity of the target protein.

In some embodiment, the target gene is selected from the group consisting of:

| Disease | Target gene |
|---|---|
| Achondroplasia | FGFR3 |
| acute intermittent porphyria | PBGD |
| Alpha-1 antitrypsin deficiency | SERPINA1 |
| Alport syndrome | COL4A3 or COL4A4 |
| Amyotrophic lateral sclerosis | C9orf72, SOD1, TARDBP, FUS, ALS2, ANG, ATXN2, |
| | CHCHD10, CHMP2B, DCTN1, ERBB4, FIG4, HNRNPA1, |
| | MATR3, NEFH, OPTN, PFN1, PRPH, SETX, SIGMAR1, |
| | SMN1, SPG11, SQSTM1, TBK1, TRPM7, TUBA4A, |
| | UBQLN2, VAPB or VCP |
| autoimmune lymphoproliferative | CTLA4 |
| syndrome type V | |
| autosomal dominant anhidrotic | NFKBIA |
| ectodermal dysplasia with T-cell | |
| immunodeficiency | |
| Autosomal dominant congenital | RHO, GNAT1 or PDE6B |
| stationary night blindness | |
| Autosomal dominant hyper-IgE | STAT3 |
| syndrome | |
| Charcot-Marie-Tooth | PMP22, MPZ, LITAF, EGR2, NEFL, MFN2, KIF1B, |
| | RAB7A, LMNA, TRPV4, BSCL2, GARS, HSPB1, MPZ, |
| | GDAP1, HSPB8, DNM2, YARS, GJB1 or PRPS1 |
| Chronic Mucocutaneous | STAT1 |
| Candidiasis | |
| Common variable immune | NFKB2 |
| deficiency 10 | |
| Common variable immune | NFKB1 |
| deficiency 12 | |
| Common variable immune | IKZF1 |
| deficiency 13 | |
| Common variable immune | TNFRSF13B |
| deficiency 2 | |
| Congenital hyperinsulinism | ABCC8, KCNJ11, GLUD1, HADH, HNF1A, HNF4A, |
| | SLC16A1 or UCP2 |
| Cowden syndrome | PTEN, SDHB, SDHD, or KLLN |
| Denys-Drash syndrome | WT1 |
| Diffuse-type gastric carcinoma | RHOA |
| dyskeratosis congenita-1 | TERC |
| Dystonia 6 | THAP1 |
| dystrophic epidermolysis bullosa | COL7A1 |
| pruriginosa | |
| Early-onset primary dystonia | TOR1A |
| Ehlers-Danlos syndrome type IV | COL3A1 |
| Ehlers-Danlos syndrome type VII | COL1A1 or COL1A2 |
| epidermolysis bullosa dystrophica | COL7A1 |
| epidermolysis bullosa simplex | KRT5, KRT15, PLEC1 or ITGB4 |
| Familial adenomatous polyposis | APC |
| familial breast-ovarian cancer-1 | BRCA1 |
| familial retinoblastoma | RB1 |
| Fragile X syndrome | FMR1 |
| Hereditary hemochromatosis | SLC40A1 |
| type 4 | |
| Hereditary hemorrhagic | ACVRL1, ENG or SMAD4 |
| telangiectasia | |
| Hereditary leiomyomatosis and | FH |
| renal cell cancer | |
| Hereditary prostate cancer | BRCA1, BRCA2 or HOXB13 |
| hereditary spastic paraplegia | REEP1 |
| type 31 | |
| hereditary spastic paraplegia | ATL1 |
| type 3A | |
| hereditary spastic paraplegia | SPAST |
| type 4 | |
| hereditary spastic paraplegia | WASHC5 |
| type 8 | |
| Hereditary spherocytosis | ANK1, EPB42, SLC4A1, SPTa1 or SPTB |
| Huntington disease | HTT |
| hyper-IgE recurrent infection | STAT3 |
| syndrome | |
| Hypercholesterolemia | LDLR, APOB or PCSK9 |
| Hyperkalemic periodic paralysis | SCN4A |
| Hypokalemic periodic paralysis | CACNA1S or SCN4A |
| immunodeficiency-13 | UNC119 |
| immunodeficiency-14 | PIK3CD |
| immunodeficiency-21 | GATA2 |
| immunodeficiency-27B | IFNGR1 |
| immunodeficiency-31A | STAT1 |
| immunodeficiency-31C | STAT1 |
| immunodeficiency-32A | IRF8 |
| immunodeficiency-36 | PIK3R1 |
| immunodeficiency-45 | IFNAR2 |
| immunodeficiency-49 | BCL11B |
| Immunoglobulin A (IgA) | TNFRSF13B |
| deficiency-2 | |
| Incontinentia pigmenti | IKBKG |
| Infantile-onset spinocerebellar | TWNK |
| ataxia | |
| Li-Fraumeni syndrome | TP53 or CHEK2 |
| Lynch syndrome | MLH1, MSH2, MSH6, PMS2 or EPCAM |
| Marfan syndrome | FBN1 |
| maturity-onset diabetes of the | HNF4A, GCK, HNF1A, PDX1, TCF2, NEUROD1, KLF11, |
| young | CEL, PAX4, INS, BLK, KCNJ11 or APPL1 |
| mental retardation-43 | HIVEP2 |
| Multiple endocrine neoplasia | MEN1, RET or CDKN1B |
| Multiple exostoses type I | EXT1 |
| Multiple exostoses type II | EXT2 |
| Myoclonus-dystonia | SGCE |
| Myotonic dystrophy | DMPK or CNBP |
| Neurofibromatosis type 1 | NF1 |
| Neurofibromatosis type 2 | NF2 |
| neutropenia-1 | ELANE |
| nevoid basal cell carcinoma | PTCH1 |
| syndrome | |
| Osteogenesis imperfecta | COL1A1, COL1A2, CRTAP or P3H1 |
| Peutz-Jeghers syndrome | STK11 |
| Polycystic kidney disease | PKD1 or PKD2 |
| Rapid-onset dystonia parkinsonism | ATP1A3 |
| Retinitis pigmentosa | RHO, RP1, PRPH2RP9, IMPDH1, PRPF31, PRPF8, CA4, |
| | PRPF3, ABCA4, NRL, FSCN2, TOPORS, SNRNP200, |
| | SEMA4A, NR2E3, KLHL7, RGR, GUCA1B, BEST1, PRPF6 |
| | or PRPF4 |
| sickle cell disorder | beta-globin, BCL11A |
| Spinal muscular atrophy, lower | VAPB |
| extremity, dominant (SMA-LED) | |
| and adult-onset form of spinal | |
| muscular atrophy | |
| Spinocerebellar ataxia type 1 | ATXN1 |
| Spinocerebellar ataxia type 2 | ATXN2 |
| Spinocerebellar ataxia type 3 | ATXN3 |
| Spinocerebellar ataxia type 36 | NOP56 |
| Spinocerebellar ataxia type 6 | CACNA1A |
| Tuberous sclerosis complex | SC1 or TSC2 |
| Von Hippel-Lindau syndrome | VHL |
| Von Willebrand disease type I and | VWF |
| II | |

In a preferred embodiment, the recombinant viral vector further comprises the elements 1, 2, 3, 4 and 5 below, or elements 1, 2, 3, 4, 5, and 6 below:
1) An expression cassette encoding the protein that has a therapeutic effect;
2) A self-inactivating (SIN) LTR configuration;
3) A packaging signal;
4) A Rev Responsive Element (RRE) to enhance nuclear export of unspliced recombinant viral vector RNA;
5) A central polypurine tract (cPPT) to enhance nuclear import of recombinant viral vector genomes; and
6) A post-transcriptional regulatory element (PRE) to enhance recombinant viral vector genome stability and to improve recombinant viral vector titers (e.g., WPRE).

As indicated above, in various embodiments the recombinant viral vector described herein comprises an expression cassette encoding the protein that has a therapeutic effect, under the control of tissue-specific or ubiquitous transcriptional control elements (e.g. promoter or enhancer) able to ensure the expression of the therapeutic protein in the disease target cells. For example, the expression cassette encodes a beta-like globin gene (i.e. gamma-globin, beta-globin, delta-globin. For example, the expression cassette encodes a human gamma-globin gene, for example the expression cassette comprises ˜1.95 kb recombinant human gamma-beta-globin gene (i.e. gamma-globin exons and beta-globin introns, where beta-globin intron 2 has a 600-bp RsaI to SspI deletion) under the control of transcriptional control elements (e.g., the human beta-globin gene promoter (e.g., −265 bp/+50 bp)), and a 2.7 kb composite human beta-globin locus control region (e.g., HS2 −1203 bp; HS3 −1213 bp and/or HS4 −954 bp).

The beta-like globin gene (gamma-globin, beta-globin, delta-globin,) cassette, however, is illustrative and need not be limiting. Using the known cassette described herein, numerous variations will be available to one of skill in the art. Such variations include, for example, further and/or alternative mutations to the beta-globin to further enhance non-sickling properties (e.g., PAS3 cassette is described by Levasseur (2003) Blood 102: 4312-4319), alterations in the transcriptional control elements (e.g., promoter and/or enhancer such as HS4), variations on the intron size/structure, and the like. In a preferred embodiment the cassette lacks HS4 (i.e. the recombinant viral vector lacks HAS). The inventors showed that the absence of HS4 increases recombinant viral vector titer and therefore efficiency and efficacy of the recombinant viral vector; and the absence of HS4 does not affect the therapeutic potential of the recombinant viral vectors.

### Self Inactivating (SIN) LTR Configuration

To further improve safety, in various embodiments, the recombinant lentiviral vectors described herein comprise a TAT-independent, self-inactivating (SIN) configuration. Thus, in various embodiments it is desirable to employ in the LVs described herein an LTR region that has reduced promoter activity relative to wild-type LTR. Constructs can be provided that are effectively “self-inactivating” (SIN), which provides a biosafety feature. SIN vectors are ones in which the production of full-length recombinant viral vector RNA in transduced cells is greatly reduced or abolished altogether. This feature minimizes the risk that replication-competent recombinants (RCRs) will emerge. Furthermore, it reduces the risk that that cellular coding sequences located adjacent to the recombinant viral vector integration site will be aberrantly expressed. The SIN configurations are well known in the art.

### Packaging Signal

In various embodiments the recombinant viral vectors described herein further comprise a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art. In a specific embodiment, the packaging sequence is the naturally occurring packaging sequences.

### Rev Responsive Element (RRE).

In certain embodiments the recombinant viral vectors described herein comprise a Rev Response Element (RRE) to enhance nuclear export of unspliced RNA. RREs are well known to those of skill in the art.

### Expression-Stimulating Posttranscriptional Regulatory Element (PRE)

In certain embodiments the recombinant viral vectors described herein may comprise any of a variety of posttranscriptional regulatory elements (PREs) whose presence within a transcript increases expression of the heterologous nucleic acid (e.g., gamma-beta-globin gene) at the protein level. PREs may be particularly useful in certain embodiments, especially those that involve viral constructs with poorly efficient promoters.

One type of PRE is an intron positioned within the expression cassette, which can stimulate gene expression. However, introns can be spliced out during the life cycle events of a lentivirus. Hence, if introns are used as PREs they are typically placed in an opposite orientation to the recombinant viral vector genomic transcript. PREs are well known to those of skill in the art.

The invention also relates to a composition comprising a recombinant viral vector of the invention or a plurality of recombinant viral vectors of the invention. The recombinant viral vector or a plurality of recombinant viral vectors of the invention can be purified to become substantially pure. The phrase “substantially pure” means that the recombinant viral vectors contain substantially no replicable virus other than the recombinant viral vectors. The purification can be achieved using known purification and separation methods such as filtration, centrifugation and column purification. If necessary, the recombinant viral vector or a plurality of recombinant viral vectors of the invention can be prepared as compositions by appropriately combining them with desired pharmaceutically acceptable carriers or vehicles. The term “pharmaceutically acceptable carrier” refers to a material that can be added to the recombinant viral vector or the plurality of recombinant viral vectors of the invention and does not significantly inhibit recombinant viral vector-mediated gene transfer. Specifically, the recombinant viral vector or the plurality of recombinant viral vectors can be appropriately combined with, for example, sterilized water, physiological saline, culture medium, serum, and phosphate buffered saline (PBS). The recombinant viral vector or the plurality of recombinant viral vectors can also be combined with a stabilizer, biocide, etc. Compositions containing a recombinant viral vector or a plurality of recombinant viral vectors of the present invention are useful as reagents or pharmaceuticals. For example, compositions of the present invention can be used as reagents for gene transfer into a cell, preferably for transduction of a cell, in particular a stem cell, more particularly a human stem cell.

The invention also relates to a kit of parts comprising:
a recombinant viral vector of the invention or a composition of the invention; and
a catalytically active Cas9 or Cpf1 protein or a nucleotide sequence encoding a catalytically active Cas9 or Cpf1 protein.

According to the invention, a complex gRNA/Cas9 or gRNA/Cpf1 induces the target nucleotide sequence to be disrupted and/or new ones added through a system called “CRISPR/Cas9 system” or “CRISPR/Cpf1 system”. CRISPR means Clustered Regularly Interspaced Short Palindromic Repeats.

The CRISPR/Cas system is a prokaryotic immune system that confers resistance to foreign genetic elements such as those present within plasmids and phages, and provides a form of acquired immunity. CRISPR associated proteins (Cas), e.g. Cas9 use the CRISPR spacers to recognize and cut a target nucleotide sequence. By delivering into a cell the Cas9 and gRNA that comprises a spacer adapted to bind to a target nucleotide sequence, the cell genome can be cut at a desired location, inducing a target nucleotide sequence to be removed and/or new ones added (Mandal et al., Cell Stem Cell, 2014, 15(5):643-52). The term “Cas9” comprises Cas9 variants such as saCas9, spCAS9, esp-CAS9 or spCas9-HF1.

In a specific embodiment of the invention, said target nucleotide sequence is within the coding sequence of a target gene, within a transcribed non-coding sequence of a target gene or within a non-transcribed sequence, either upstream or downstream of a target gene. Therefore, the complex gRNA/Cas9 or gRNA/Cpf1 may disrupt (e.g. may knock-out of) the expression and/or the function of the target gene.

In some embodiment, the target gene is involved in a genetic disorder when said target gene is expressed in a patient. In some embodiment, the target gene may be selected from the group consisting of:

| Disease | Target gene |
|---|---|
| Achondroplasia | FGFR3 |
| acute intermittent porphyria | PBGD |
| Alpha-1 antitrypsin deficiency | SERPINA1 |
| Alport syndrome | COL4A3 or COL4A4 |
| Amyotrophic lateral sclerosis | C9orf72, SOD1, TARDBP, FUS, ALS2, ANG, ATXN2, |
| | CHCHD10, CHMP2B, DCTN1, ERBB4, FIG4, |
| | HNRNPA1, MATR3, NEFH, OPTN, PFN1, PRPH, SETX, |
| | SIGMAR1, SMN1, SPG11, SQSTM1, TBK1, TRPM7, |
| | TUBA4A, UBQLN2, VAPB or VCP |
| autoimmune lymphoproliferative | CTLA4 |
| syndrome type V | |
| autosomal dominant anhidrotic | NFKBIA |
| ectodermal dysplasia with T-cell | |
| immunodeficiency | |
| Autosomal dominant congenital | RHO, GNAT1 or PDE6B |
| stationary night blindness | |
| Autosomal dominant hyper-IgE | STAT3 |
| syndrome | |
| Charcot-Marie-Tooth | PMP22, MPZ, LITAF, EGR2, NEFL, MFN2, KIF1B, |
| | RAB7A, LMNA, TRPV4, BSCL2, GARS, HSPB1, MPZ, |
| | GDAP1, HSPB8, DNM2, YARS, GJB1 or PRPS1 |
| Chronic Mucocutaneous Candidiasis | STAT1 |
| Common variable immune | NFKB2 |
| deficiency 10 | |
| Common variable immune | NFKB1 |
| deficiency 12 | |
| Common variable immune | IKZF1 |
| deficiency 13 | |
| Common variable immune | TNFRSF13B |
| deficiency 2 | |
| Congenital hyperinsulinism | ABCC8, KCNJ11, GLUD1, HADH, HNF1A, HNF4A, |
| | SLC16A1 or UCP2 |
| Cowden syndrome | PTEN, SDHB, SDHD, or KLLN |
| Denys-Drash syndrome | WT1 |
| Diffuse-type gastric carcinoma | RHOA |
| dyskeratosis congenita-1 | TERC |
| Dystonia 6 | THAP1 |
| dystrophic epidermolysis bullosa | COL7A1 |
| pruriginosa | |
| Early-onset primary dystonia | TOR1A |
| Ehlers-Danlos syndrome type IV | COL3A1 |
| Ehlers-Danlos syndrome type VII | COL1A1 or COL1A2 |
| epidermolysis bullosa dystrophica | COL7A1 |
| epidermolysis bullosa simplex | KRT5, KRT15, PLEC1 or ITGB4 |
| Familial adenomatous polyposis | APC |
| familial breast-ovarian cancer-1 | BRCA1 |
| familial retinoblastoma | RB1 |
| Fragile X syndrome | FMR1 |
| Hereditary hemochromatosis type 4 | SLC40A1 |
| Hereditary hemorrhagic | ACVRL1, ENG or SMAD4 |
| telangiectasia | |
| Hereditary leiomyomatosis and | FH |
| renal cell cancer | |
| Hereditary prostate cancer | BRCA1, BRCA2 or HOXB13 |
| hereditary spastic paraplegia type | REEP1 |
| 31 | |
| hereditary spastic paraplegia type | ATL1 |
| 3A | |
| hereditary spastic paraplegia type 4 | SPAST |
| hereditary spastic paraplegia type 8 | WASHC5 |
| Hereditary spherocytosis | ANK1, EPB42, SLC4A1, SPTa1 or SPTB |
| Huntington disease | HTT |
| hyper-IgE recurrent infection | STAT3 |
| syndrome | |
| Hypercholesterolemia | LDLR, APOB or PCSK9 |
| Hyperkalemic periodic paralysis | SCN4A |
| Hypokalemic periodic paralysis | CACNA1S or SCN4A |
| immunodeficiency-13 | UNC119 |
| immunodeficiency-14 | PIK3CD |
| immunodeficiency-21 | GATA2 |
| immunodeficiency-27B | IFNGR1 |
| immunodeficiency-31A | STAT1 |
| immunodeficiency-31C | STAT1 |
| immunodeficiency-32A | IRF8 |
| immunodeficiency-36 | PIK3R1 |
| immunodeficiency-45 | IFNAR2 |
| immunodeficiency-49 | BCL11B |
| Immunoglobulin A (IgA) | TNFRSF13B |
| deficiency-2 | |
| Incontinentia pigmenti | IKBKG |
| Infantile-onset spinocerebellar | TWNK |
| ataxia | |
| Li-Fraumeni syndrome | TP53 or CHEK2 |
| Lynch syndrome | MLH1, MSH2, MSH6, PMS2 or EPCAM |
| Marfan syndrome | FBN1 |
| maturity-onset diabetes of the | HNF4A, GCK, HNF1A, PDX1, TCF2, NEUROD1, KLF11, |
| young | CEL, PAX4, INS, BLK, KCNJ11 or APPL1 |
| mental retardation-43 | HIVEP2 |
| Multiple endocrine neoplasia | MEN1, RET or CDKN1B |
| Multiple exostoses type I | EXT1 |
| Multiple exostoses type II | EXT2 |
| Myoclonus-dystonia | SGCE |
| Myotonic dystrophy | DMPK or CNBP |
| Neurofibromatosis type 1 | NF1 |
| Neurofibromatosis type 2 | NF2 |
| neutropenia-1 | ELANE |
| nevoid basal cell carcinoma | PTCH1 |
| syndrome | |
| Osteogenesis imperfecta | COL1A1, COL1A2, CRTAP or P3H1 |
| Peutz-Jeghers syndrome | STK11 |
| Polycystic kidney disease | PKD1 or PKD2 |
| Rapid-onset dystonia parkinsonism | ATP1A3 |
| Retinitis pigmentosa | RHO, RP1, PRPH2RP9, IMPDH1, PRPF31, PRPF8, |
| | CA4, PRPF3, ABCA4, NRL, FSCN2, TOPORS, |
| | SNRNP200, SEMA4A, NR2E3, KLHL7, RGR, GUCA1B, |
| | BEST1, PRPF6 or PRPF4 |
| sickle cell disorder | beta-globin, BCL11A |
| Spinal muscular atrophy, lower | VAPB |
| extremity, dominant (SMA-LED) and | |
| adult-onset form of spinal muscular | |
| atrophy | |
| Spinocerebellar ataxia type 1 | ATXN1 |
| Spinocerebellar ataxia type 2 | ATXN2 |
| Spinocerebellar ataxia type 3 | ATXN3 |
| Spinocerebellar ataxia type 36 | NOP56 |
| Spinocerebellar ataxia type 6 | CACNA1A |
| Tuberous sclerosis complex | SC1 or TSC2 |
| Von Hippel-Lindau syndrome | VHL |
| Von Willebrand disease type I and | VWF |
| II | |

It is well known that, CRISPR/Cas9 system, when utilized for genome editing, may include Cas9, CRISPR RNA (crRNA) and/or trans-activating crRNA (tracrRNA):
crRNA comprises the RNA that binds to a target nucleotide sequence, said RNA is along with a tracrRNA (generally in a hairpin loop form);
tracrRNA and crRNA form an active complex, named guide RNA (gRNA). Because eukaryotic systems lack some of the proteins required to process crRNA, the synthetic construct gRNA was created to combine the essential pieces of RNA for Cas9 targeting into a single RNA. Commonly, the gRNA is expressed with the RNA polymerase type III promoter U6 (promoter U6);
Cas9 is a nuclease protein whose active form is able to modify DNA. Many variants exist with differing functions (i.e. single strand nicking, double strand break, DNA binding) due to Cas9's DNA site recognition function. In a preferred embodiment of the invention, Cas9 has a double strand break function. The term “Cas9” comprises Cas9 variants. Among the variants we can list, but not limited to, spCAS9, esp-CAS9, spCas9-HF1.

The nucleic acid cleavages caused by Cas9 or Cpf1 are commonly repaired through the distinct mechanisms of homologous recombination or non-homologous end joining (NHEJ). NHEJ is an imperfect repair process that often results in changes to the nucleotide sequence at the site of the cleavage (i.e. the target nucleotide sequence). Repair via non-homologous end joining (NHEJ) often results in small insertions or deletions and can be used for the creation of specific gene knock-outs.

In one aspect of the present invention, CRISPR/Cas9 or CRISPR/Cpf1 system modifies the genome of an eukaryotic cell, preferably an eukaryotic stem cell, e.g. a human stem cell. Thus, in one aspect of the present invention, CRISPR/Cas9 or CRISPR/Cpf1 system aims to induce knock-out of a target nucleotide sequence in the transduced eukaryotic cell, and therefore to disrupt (e.g. to induce a knock-out of) the target gene in the transduced eukaryotic cell, and therefore to disrupt (e.g. to suppress) the expression and/or the activity of the target protein in the transduced eukaryotic cell and/or in the differentiated progeny of the transduced eukaryotic cell.

The invention also relates to the use of a recombinant viral vector of the invention or a composition of the invention for introducing into a cell (i) nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder and (ii) a nucleotide sequence encoding a protein that has a therapeutic effect in said genetic disorder. In some embodiment, the use is in vitro, ex vivo or in vivo.

The invention also relates to a method for modifying the genome of a cell in vitro, ex vivo or in vivo, comprising the steps of:
a) contacting the cell with a recombinant viral vector of the invention or a composition of the invention to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

The invention also relates to a method for preparing a genetically modified cell in vitro, ex vivo or in vivo, comprising the steps of:
a) contacting the cell with a recombinant viral vector of the invention or a composition of the invention to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

The term “transduction”, according to the invention, means the process by which a foreign nucleotide sequence is introduced into the genome of a cell by a recombinant viral vector. According to the invention, a cell transduced by the recombinant viral vector of the invention, also referred as a “transduced cell”, encodes (i.e. comprises in its genome) the nucleotide sequence encoding a protein that has a therapeutic effect and the nucleotide sequence encoding a gRNA that comprises a spacer adapted to bind to a target nucleotide sequence. Thus, according to a preferred embodiment, a transduced cell expresses the protein that has a therapeutic effect and the gRNA that comprises a spacer adapted to bind to a target nucleotide sequence.

The methods of the invention involve introducing a catalytically active Cas9 or Cpf1 protein (hereafter “Cas9” or “Cpf1”) or a nucleotide sequence encoding Cas9 or Cpf1, preferably a RNA encoding Cas9 or Cpf1, into the transduced cell. The following paragraphs only refer to “Cas9”, however, “Cas9” can be replaced by “Cfp1”.

According to the invention, Cas9 can be optimized for the organism in which it is being introduced. Thus, for example Cas9 polynucleotide sequence derived from the pyogenes or S. Thermophilus codon optimized for use in human is set forth in Cong et al., Science, 2013, 339(6121):819-23; Mali et al., Science, 2013, 339(61210):823-6; Kleinstiver et al., Nature, 2015, 523(7561):481-5; Hou et al., Proc Natl Acad Sci USA, 2013, 110(39):11644-9; Ran et al., Nature, 2015, 520(7546):186-191.

Cas9 may be directly introduced into the transduced cell as a protein or may be synthesized (or expressed) in situ in the cell as a result of the introduction of a nucleotide sequence encoding Cas9, for example a DNA or a RNA encoding Cas9, preferably a RNA encoding Cas9.

Cas9 or a nucleotide sequence encoding Cas9 can be produced outside the cell and then introduced thereto.

Methods for introducing a nucleotide sequence into cells are known in the art and including, as non-limiting examples, stable transduction methods wherein the nucleotide sequence is integrated into the genome of the cell (recombinant viral vector-mediated methods) or transient transfection methods wherein the nucleotide sequence is not integrated into the genome of the cell (recombinant viral vector-mediated methods, liposomes, microinjection, electroporation, particle bombardment and the like). Said nucleotide sequence may be included in a vector, more particularly a plasmid or a viral vector, in view of being expressed in the cells. In a preferred embodiment, the method for introducing a nucleotide sequence encoding Cas9 into cells is a transient transfection method.

In a specific embodiment, the nucleotide sequence encoding Cas9 is a DNA encoding Cas9. In this embodiment, the transient transfection is particularly advantageous because the DNA sequence encoding Cas9 is not integrated into the genome of the cell and therefore Cas9 is thus produced transiently in a limited period of time. After the transient production, given that the cell does not comprise in its genome a nucleotide sequence encoding Cas9, the cell does not produce Cas9 anymore. This is particularly advantageous when the cell is then used as a medicament in ex vivo treatments. Furthermore, the rapid gRNA degradation in absence of Cas9 nuclease will avoid interferon response and apoptosis, therefore improving safety issues.

In another specific embodiment, the nucleotide sequence encoding Cas9 is a RNA encoding Cas9. The RNA also has the advantage of not being integrated into the genome of the cell. For example, a RNA encoding Cas9 is introduced by electroporation or liposomes.

Methods for introducing a protein into cells are known in the art and include as non-limiting examples the use of liposomes, microinjection, electroporation or particle bombardment. For example, Cas9 is introduced into the cell by electroporation or liposomes.

In a particular embodiment, Cas9 is introduced into the cell as a protein. In this embodiment, Cas9 has the advantage of not being integrated into the genome of the cell and to be rapidly degraded. For example, Cas9 is introduced by electroporation or nanoparticles.

According to the invention, Cas9 may form a complex with the gRNA in the transduced HSPC. Said Cas9/gRNA complex may bind to the target nucleotide sequence and may therefore disrupt the expression or the function of the target gene. In a preferred embodiment, the Cas9/gRNA complex induces a knock-out of the expression or the function of the target gene. In a specific embodiment, the methods of the invention are particularly advantageous because the only cells that are able to survive after the disruption of the target gene are those that comprise in their genome the nucleotide sequence encoding the protein that has a therapeutic effect and that express said protein that has a therapeutic effect. In this specific embodiment, the protein that has a therapeutic effect is needed by the cell to survive after the disruption of the target gene.

In a particular embodiment, the cell is an eukaryotic cell, preferably a mammalian cell, preferably a human cell. In some embodiments, for in vivo approaches, the cells are stem cells, progenitor cells or differentiated cells. In some embodiments, for ex-vivo and in vitro approaches, the cells are a stem cell, e.g. a human stem cell, progenitor cells or differentiated cells, e.g. T lymphocytes.

The invention also relates to a genetically modified cell obtainable by the methods according the invention and said genetically modified cell for use as a medicament.

In one embodiment, the invention relates to a genetically modified cell obtainable by the methods according the invention for use in the treatment of a disorder, in particular an autosomal dominant disorders which require the alteration (e.g. disruption) of a dominant allele or a recessive genetic disorder in which the expression of an endogenous mutated protein compromise the beneficial effects induced by the expression of an exogenous corrected protein (e.g. sickle cell disease).

In another embodiment, the invention relates to a genetically modified cell obtainable by the methods according the invention for use in the treatment of an autosomal dominant blood disorder, in particular an autosomal dominant blood disorder which requires the alteration (e.g. disruption) of the dominant allele. In some embodiments, the autosomal dominant blood disorder is selected from the group consisting of a primary immunodeficiency, neutropenia-1, hyper-IgE recurrent infection syndrome, Hereditary spherocytosis. In some embodiment, the primary immunodeficiency is selected from the group consisting of immunodeficiency-13, immunodeficiency-14, immunodeficiency-21, immunodeficiency-27B, immunodeficiency-31A, immunodeficiency-31C, immunodeficiency-32A, immunodeficiency-36, immunodeficiency-45, immunodeficiency-49 and immunoglobulin A (IgA) deficiency-2.

In another embodiment, the invention relates to a genetically modified cell obtainable by the methods according the invention for use in the treatment of a hemoglobinopathy, in particular sickle cell disease or disorder (SCD).

In one embodiment, the cell is a human stem cell, e.g. a human HSC, or a differentiated cell, e.g. T lymphocyte, can be removed from a human, e.g. a human patient, using methods well known to those of skill in the art and modified as noted above. The genetically modified cell is then reintroduced into the same or a different human, preferably the same human. The human stem cell may be obtained from the bone marrow, the peripheral blood or the umbilical cord blood. Particularly preferred human stem cells are CD34+ cells.

Accordingly, the invention also relates to a method of treating a genetic disorder in a patient comprising the steps of:
a) obtaining a cell from the patient;
b) contacting the cell with a recombinant viral vector of the invention or a composition of the invention to obtain a transduced cell;
c) introducing a catalytically active Cas9 or Cpf1 protein or a nucleotide sequence encoding a catalytically active Cas9 or Cpf1 protein into the transduced cell, said catalytically active Cas9 or Cpf1 protein disrupts the expression and/or the function of the target gene when introduced or expressed into the transduced cell, to obtain a genetically modified cell;
d) administrating the genetically modified cell into the patient.

In some embodiments, the administration may be a transplantation or an inoculation, in particular a transplantation or an inoculation in the bone narrow.

According to the invention, when the protein that has a therapeutic effect is a functional version (e.g. the wild-type version) of the target protein, the design of the nucleotide(s) sequence(s) (e.g. the nucleotide sequence encoding the protein that has a therapeutic effect and/or the nucleotide sequence encoding the gRNA) will be easily adapted by the skilled person in order to avoid that the gRNA targets the nucleotide sequence encoding the protein that has a therapeutic effect (i.e. codon design). For example, the recombinant viral vector comprises a nucleotide sequence encoding beta-globin (e.g. PAS3 beta-globin cassette, described by Levasseur et al., Blood, 2003, 102(13):4312-9) and a nucleotide sequence encoding a gRNA targeting the sickle beta-globin. In this case, to avoid the unwanted disruption of the nucleotide sequence encoding beta-globin (i.e. the beta-globin transgene), the nucleotide sequence encoding beta-globin will be modified introducing silent mutations in the transgene sequence, so that it will not be recognized by the gRNA (see FIG. 14). To this aim, the skilled person commonly uses synonymous codons (coding for the same amino acids), allowing the change of the nucleotide sequence and the production of an identical beta-globin protein. Generally, synonymous codons will be chosen amongst the most frequently used codons in the beta- and alpha-globin genes.

### FIGURES

FIG. 1: Construction of a recombinant lentiviral vector encoding a beta-like globin gene

FIG. 2: Evaluation of genome editing efficiency in hematopoietic cells using the CRISPR-Cas9 system

FIG. 3: Construction and screening of a gRNA for beta-globin gene inactivation: design of gRNAs targeting HBB gene.

FIG. 4: Selection of gRNAs targeting the beta-globin gene: design of novel gRNAs

FIG. 5: Cleavage efficiency of gRNAs A, B, D and E in K562 and HUDEP-2 erythroid cell lines

FIG. 6: Down regulation of beta-globin expression in HUDEP-2

FIG. 7: Cleavage efficiency of selected gRNA (B, D and E) in HSPCs

FIG. 8: Down regulation of beta-globin expression in HSPC-derived erythroid cells

FIG. 9: Optimization of gRNA-mediated disruption of the target site

FIG. 10: Construction of a recombinant lentiviral vector according to the invention

FIG. 11: Transduction of HSPC with a recombinant lentiviral vector according to the invention and introduction of Cas9 into the transduced cell.

FIG. 12: Genetic modification of patient SCD HSPC in vitro

FIG. 13: Genetic modification of patient SCD HSC in vivo

FIG. 14: nucleotide sequences encoding globin variants that have a therapeutic effect according to the invention. The gRNA D target site is underlined. The nucleotides changes in the Beta AS3 (modified to avoid targeting by gRNA D) and Beta AS1 (T87Q) (modified to avoid targeting by gRNA D) transgenes are highlighted in grey/green.

FIG. 15: Assessment of globin mRNAs expression in mature erythroblasts (day 9 of differentiation) derived from control and genetically modified HUDEP-2 cell lines. UT: mature erythroblasts derived from non-transduced and non-transfected HUDEP-2 cells: “normal” level of globin β, δ and γ globin (negative control); VCN: «vector copy number»; Not transfected: mature erythroblasts derived from non-transfected HUDEP-2 cells; GFP+ (Cas9 plasmid): mature erythroblasts derived from HUDEP-2 cells expressing Cas9-GFP fusion protein, selected by FACS upon transfection with GFP-Cas9 plasmid; Cas9 protein: mature erythroblasts derived from HUDEP-2 cells transfected with Cas9-GFP protein without using selection-based strategies; when transduced, cells were treated with a lentiviral vector expressing beta-globin AS3mod transgene and a gRNA selected from: “D” lentiviral vector encoding optimized gRNA D, “luc” lentiviral vector encoding an optimized gRNA targeting the luciferase gene, which is not present in the human genome (negative control), “BCL11A” lentiviral vector encoding an optimized gRNA targeting the intronic erythroid-specific enhancer of BCL11A gene, “13bpdel” lentiviral vector encoding a gRNA designed to reproduce the 13 bp small HPFH deletion within the promoters of HBG1 and HBG2 genes; β: endogenous beta-globin mRNA; β-AS3: AS3 beta-globin transgene mRNA; Aγ+Gγ: gamma-globin mRNA; δ: delta-globin mRNA.

FIG. 16: Reverse phase HPLC profile of single globin chains in mature erythroblasts (day 9 of differentiation) derived from control and genetically modified HUDEP-2 cell lines. (A) mature erythroblasts derived from WT (wild-type) HUDEP-2 UT cells: not transduced and not transfected cells expressing “normal” level of globin β, δ and γ globin (negative control); (B) mature erythroblasts derived from HUDEP-2 cells transduced with LV.GLOBE.AS3mod-beta-globin.gRNA D (lentiviral GLOBE vector encoding the AS3modified beta-globin and the optimized gRNA D) but not transfected with Cas9-GFP plasmid: cells express the AS3modified beta-globin transgene and the endogenous beta-globin chain (no modification of the endogenous HBB gene); (C) mature erythroblasts derived from HUDEP-2 cells transduced cells with the LV.GLOBE.AS3mod-beta-globin.gRNA D (lentiviral GLOBE vector encoding the AS3modified beta-globin and the optimized gRNA D) and transfected with the GFP-Cas9 plasmid: cells express the AS3modified beta-globin transgene but not endogenous beta-globin chain because of the high rate of genome editing in the exon 1 of the endogenous HBB gene.

FIG. 17: Assessment of BCL11A mRNA expression (time-point analyses during differentiation) in HUDEP-2 cells transduced with a lentiviral vector encoding beta-globin AS3mod and a gRNA targeting the intronic erythroid-specific enhancer of BCL11A gene with (“+”) or without (“−”) transfection with Cas9-GFP plasmid.

FIG. 18: Reverse phase HPLC analysis of single globin chains in mature erythroblasts (day 9 of differentiation) derived from control and genetically modified HUDEP-2 cell lines. UT: mature erythroblasts derived from non-transduced and non-transfected HUDEP-2 cells: “normal” level of globin β, δ and γ globin (negative control); VCN: «vector copy number»; Not transfected: mature erythroblasts derived from non-transfected HUDEP-2 cells; GFP+ (Cas9 plasmid): mature erythroblasts derived from HUDEP-2 cells expressing Cas9-GFP fusion protein, selected by FACS upon transfection with GFP-Cas9 plasmid; Cas9 protein: mature erythroblasts derived from HUDEP-2 cells transfected with Cas9-GFP protein without using selection-based strategies; when transduced, cells were treated with a lentiviral vector expressing AS3mod beta-globin transgene and a gRNA selected from: “D” lentiviral vector encoding optimized gRNA D, “luc” lentiviral vector encoding an optimized gRNA targeting the luciferase gene (negative control), “BCL11A” lentiviral vector encoding an optimized gRNA targeting the intronic erythroid-specific enhancer of BCL11A gene, “13bpdel” lentiviral vector encoding a gRNA designed to reproduce the 13 bp small HPFH deletion within the promoters of HBG1 and HBG2 genes; β: endogenous beta-globin chain; β-AS3: AS3 beta-globin chain; Aγ+Gγ: gamma-globin chains; δ: delta-globin chain

FIG. 19: Cation-exchange HPLC profile of hemoglobin tetramers in mature erythroblasts (day 9 of differentiation) derived from control and genetically modified HUDEP-2 cell line (A) WT (wild-type) HUDEP-2 UT cells: mature erythroblasts derived from non-transduced and non-transfected HUDEP-2 cells: “normal” level of globin HbA (hemoglobin tetramer containing the endogenous beta-globin chain), HbA2 (hemoglobin tetramer containing the endogenous delta-globin chain) and HbF (hemoglobin tetramer containing the endogenous gamma-globin chain) (negative control); mature erythroblasts derived from HUDEP-2 cells transduced with LV.GLOBE.AS3mod-beta-globin.gRNA D (lentiviral GLOBE vector encoding the AS3modified beta-globin and the optimized gRNA D) but not transfected with Cas9-GFP plasmid: cells express the Hb tetramer containing the AS3modified beta-globin transgene (HbAS3) and HbA containing the endogenous beta-globin chain (no modification of the endogenous HBB gene); (C) mature erythroblasts derived from HUDEP-2 cells transduced with the LV.GLOBE.AS3mod-beta-globin.gRNA D (lentiviral GLOBE vector encoding the AS3modified beta-globin and the optimized gRNA D) and transfected with the GFP-Cas9 plasmid: cells express HbAS3 but not HbA because of the high rate of genome editing in the exon 1 of the endogenous HBB gene; (D) mature erythroblasts derived from HUDEP-2 cells transduced cells with the LV.GLOBE.AS3mod-beta-globin.gRNA 13 bp-del (lentiviral GLOBE vector encoding the AS3modified beta-globin and the optimized gRNA “13bpdel” encoding a gRNA designed to reproduce the 13 bp small HPFH deletion within the promoters of HBG1 and HBG2 genes) and transfected with the GFP-Cas9 plasmid: cells express the HbAS3, HbA and high levels of HbF upon genome editing of the promoters of HBG1 and HBG2 genes. HbA: α₂β₂ tetramers; HbAS3: α₂β-AS3₂ tetramers; HbA2: α₂β₂ tetramers; HbF: α₂γ₂ tetramers.

FIG. 20: Quantification of hemoglobin tetramers by HPLC, as in FIG. 19, in mature erythroblasts (day 9 of differentiation) from control and genetically modified HUDEP-2 cell line. UT: mature erythroblasts derived from non-transduced and non-transfected HUDEP-2 cells: “normal” level of globin HbA, HbA2 and HbF (negative control); VCN: «vector copy number»; Not transfected: mature erythroblasts derived from HUDEP-2 cells non-transfected with GFP-Cas9 plasmid or Cas9-GFP protein; GFP+ (Cas9 plasmid): mature erythroblasts derived from HUDEP-2 cells expressing Cas9-GFP fusion protein, selected by FACS upon transfection with GFP-Cas9; Cas9 protein: mature erythroblasts derived from HUDEP-2 cells transfected with Cas9-GFP protein without using selection-based strategies; when transduced, cells were treated with a lentiviral vector expressing beta-globin AS3mod transgene and a gRNA selected from: “D” lentiviral vector encoding optimized gRNA D, “luc” lentiviral vector encoding an optimized gRNA targeting the luciferase gene (negative control), “BCL11A” lentiviral vector encoding an optimized gRNA targeting the intronic erythroid-specific enhancer of BCL11A gene, “13bpdel” lentiviral vector encoding a gRNA designed to reproduce the 13 bp small HPFH deletion within the promoters of HBG1 and HBG2 genes. HbA: α₂β₂ tetramers; HbAS3: α₂β-AS3₂ tetramers; HbA2: α₂δ₂ tetramers; HbF: α₂γ₂ tetramers.

FIG. 21: HbF expression in mature erythroblasts (flow cytometry analysis on GPA(glycophorinA)^{high} populations) derived from control and genetically modified HUDEP-2 cells (day 9 of differentiation)

### EXAMPLES

### Example 1: Construction of a Recombinant Lentiviral Vector Encoding a Beta-Like Globin Gene

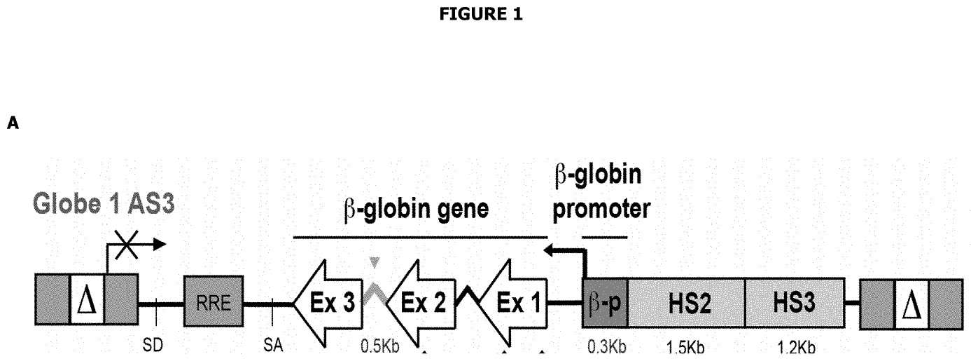

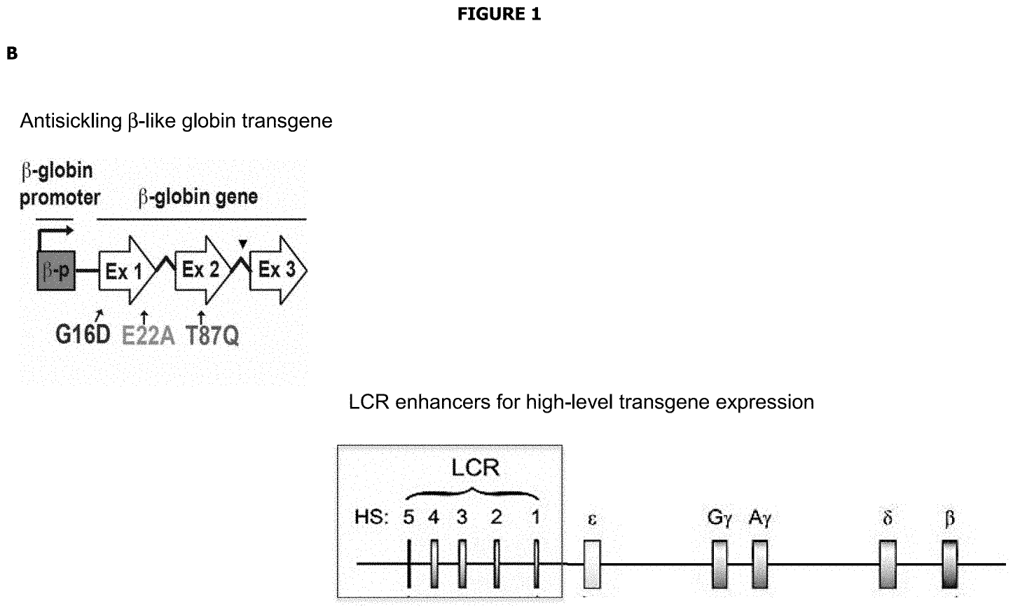

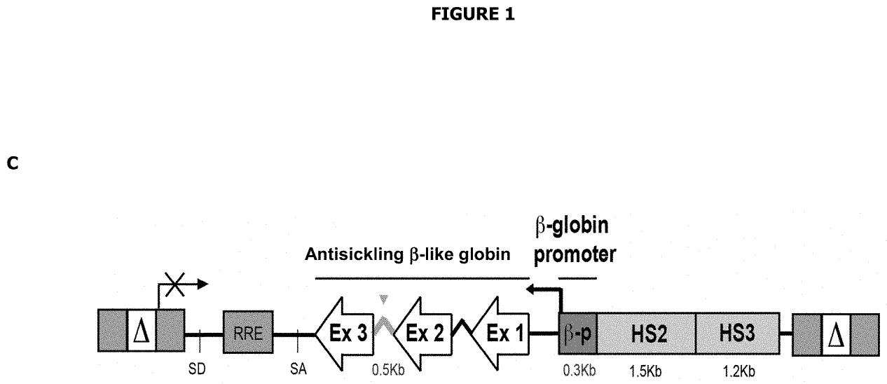

A recombinant lentiviral vector able to express at high levels a beta-like globin gene has been produced using the GLOBE lentiviral vector (Miccio et al., Proc Natl Acad Sci USA, 2008, 105(30):10547-52, Roselli et al., EMBO Mol Med, 2010, 2(8):315-28). The GLOBE lentiviral vector in its proviral form contains LTRs deleted of 400 bp in the HIV U3 region (Δ), rev-responsive element (RRE), splicing donor (SD) and splicing acceptor (SA) sites, human beta-globin gene (exons and introns), beta-globin promoter (βp), and DNase I-hypersensitive sites HS2 and HS3 from beta-globin LCR (FIGS. 1A and B). The construction of the recombinant lentiviral vector is detailed in FIG. 1C. An anti-sickling transgene (e.g. Beta AS3 (not modified), SEQ ID NO: 2; FIG. 1B) is included in the GLOBE lentiviral vector (FIG. 1C). The exons of the human beta-globin gene are replaced by exons of different anti-sickling transgenes (e.g. selected from SEQ ID NO: 1 to 8) by site-directed mutagenesis.

### Example 2: Evaluation of Genome Editing Efficiency in Hematopoietic Cells Using the CRISPR-Cas9 System

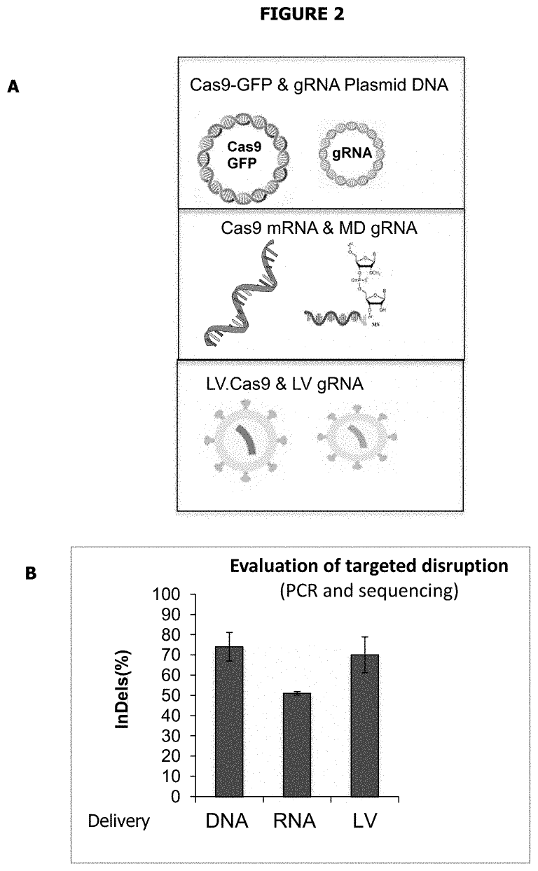

One million K562 hematopoietic cells were transfected with:
(i) 4 μg of a Cas9-GFP expressing plasmid (pMJ920, Addgene plasmid #42234) and 0.8 μg of a unrelated gRNA-expressing plasmid (MLM3636, Addgene plasmid #43860),
(ii) 20 μg of Cas9 mRNA modified with pseudouridine and 5-methylcytidine to reduce immune stimulation (Trilink, #L-6125) and 15 μg of chemically modified gRNAs (MD gRNA, 2′ O-Methyl unrelated gRNA, resistant to general base hydrolysis, Trilink); or
(iii) lentiviral vectors expressing Cas9 (Addgene, #52962) and an unrelated gRNA under the control of the human U6 promoter (FIG. 2A).
The above mentioned gRNAs were unrelated gRNAs, i.e. gRNAs binding regions which are not related to beta-globin gene or gamma-globin gene. In fact, the gRNA targets the gamma-delta intergenic region in the beta-globin locus (e.g. SEQ ID NO: 48).
K562 cells were transfected in a 100 μl volume using Nucleofector I (Lonza), the AMAXA Cell Line Nucleofector Kit V (Lonza, VCA-1003) and the T16 program.
After transfection, K562 cells were maintained in RPMI 1640 medium (Lonza) containing 2 mM glutamine and supplemented with 10% fetal bovine serum (FBS, BioWhittaker, Lonza), HEPES (20 mM, LifeTechnologies), sodium pyruvate (1 mM, LifeTechnologies) and penicillin and streptomycin (100 U/ml each, LifeTechnologies).
One week after transfection, DNA was extracted using PURE LINK Genomic DNA Mini kit (LifeTechnologies) following manufacturer's instructions. The genomic region encompassing the gRNA target site was amplified by PCR and subjected to Sanger sequencing. The genome editing efficiency (% InDels, frequency of small insertions and deletions), evaluated using TIDE (Tracking of In/Dels by Decomposition; (Brinkman et al., Nucleic Acids Res, 2014, 42(22):e168)) was higher than 50% for all the delivery systems (FIG. 2B).
These results showed that the use of DNA, RNA and lentiviral (LV) delivery systems for gRNA and Cas9 leads to a good editing efficiency in K562 hematopoietic cells.

### Example 3: Construction and Screening of a gRNA for Beta-Globin Gene Inactivation

1. Selection of gRNAs Targeting the Beta-Globin Gene

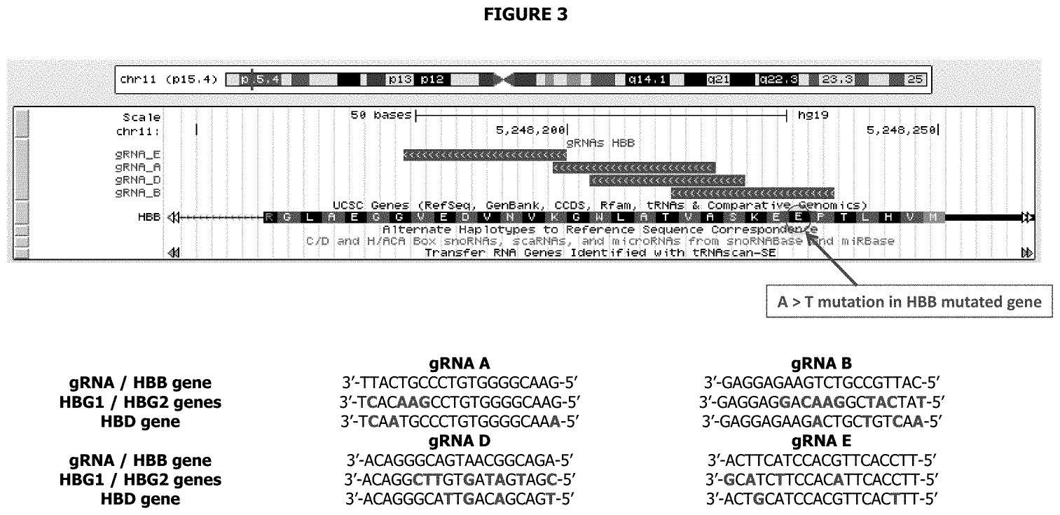

To reduce the expression of the sickle beta-globin gene (i.e. BetaS-globin gene), we selected 4 publicly available gRNAs targeting the exon 1 of the beta-globin gene (Cradick et al., Nucleic Acids Res, 2013, 41(20):9584-92; Liang et al., Protein Cell, 2015, 6(5):363-72) (gRNA spacer-encoding sequences A, B, D and E, FIG. 3, respectively SEQ ID NO: 23 to 26).

Bioinformatic prediction using COSMID (CRISPR Off-target Sites with Mismatches, Insertions, and Deletions; https://crispr.bme.gatech.edu/; Cradick et al, MolTher Nucleic Acids, 2014, 3(12):e214) showed a low number of predicted off-targets, all of them harboring ≥2 mismatches with the delta-globin target sequence (FIG. 3).

Importantly, HBG1/2 genes (coding for gamma-globins) were not included in the list of potential off-targets, the selected gRNAs displaying low similarity with the sequence of gamma-globin genes. Amongst the 4 gRNA spacers, only gRNA spacer E displays less than 3 mismatches with the sequence of exon 1 of the delta-globin gene. Bioinformatic prediction of off-target activity indicates this gene as a potential off-target of gRNA E.

The gRNA-encoding sequences A, B, D and E were cloned in MLM3636 plasmids (MLM3636, Addgene plasmid #43860), generating the following plasmids:
MLM3636 gRNA A coding for gRNA A
MLM3636 gRNA B coding for gRNA B
MLM3636 gRNA D coding for gRNA D
MLM3636 gRNA E coding for gRNA E

For the generation of MLM3636 plasmids carrying the gRNA-encoding sequences A, B, D and E, the following protocol was applied:

a. Annealing gRNA Oligos

### Oligonucleotide Sequences:

| | | SEQ |
|---|---|---|
| | | ID |
| Oligo Name | Sequence 5′ to 3′ (*) | NO: |
| Oligo FOR-gRNA A | ACACCGCTTGCCCCACAGGGCAGTAAG | 37 |
| Oligo REV-gRNA A | AAAACTTACTGCCCTGTGGGGCAAGCG | 38 |
| Oligo FOR-gRNA B | ACACCGTAACGGCAGACTTCTCCTCG | 39 |
| Oligo REV-gRNA B | AAAACGAGGAGAAGTCTGCCGTTACG | 40 |
| Oligo FOR-gNA D | ACACCGTCTGCCGTTACTGCCCTGTG | 41 |
| Oligo REV-gRNA D | AAAACACAGGGCAGTAACGGCAGACG | 42 |
| Oligo FOR-gRNA E | ACACCGAAGGTGAACGTGGATGAAGTG | 43 |
| Oligo REV-gRNA E | AAAACACTTCATCCACGTTCACCTTCG | 44 |

(*)In bold: nucleotide sequence encoding the gRNA spacer

Preparation of 10× annealing Buffer [400 μl 1M Tris HCl pH8, 200 μl 1M MgCl2, 100 μl 5M NaCl, 20 μl 0.5M EDTA pH8, 280 μl DEPC-water]. Preparation of MIX 1 for gRNA oligo annealing [1 μl 100 μM gRNA oligo FOR, 1 μl 100 μM gRNA oligo REV, 5 μl 10× annealing Buffer, 43 μl DEPC-water]. Annealing reaction in PCR machine with gradient annealing temperature: from 95° C. to 4° C. in 60 minutes, thus decreasing the annealing temperature of −1.5° C. each minute.

b. Digestion of MLM3636 Plasmid

Incubate the digestion mix reaction [x μl (2.5 μg) of MLM3636 plasmid (Addgene plasmid #43860), 5 μl of BSMB I enzyme (50 U), 5 μl of enzyme buffer 10×, (50−x) μl of DEPC-water] over-night at 55° C. Purify from low melting agarose (0.8%) gel the linearized MLM3636 plasmid (size: 2265 bp) with QIAquick Gel Extraction Kit (QIAGEN).

c. Insertion of gRNA within MLM3636 Plasmid

Incubation of ligation mix [x μl (10 ng) linearized MLM3636 plasmid, 1.1 μl of annealed gRNA-encoding sequence (diluted 1:10), 5 μl of 2× Ligase Buffer, 1 μl of Ligase (QUICK LIGASE NEB-Biolabs-M2200), (10-x) μl of DEPC-water] for 15 minutes at room temperature.

d. Transformation of Bacteria and Amplification of Plasmid

Chemical competent E. coli bacteria (One Shot TOP10 Chemically competent E. Coli-Invitrogen-C4040) are transformed with 5 μl of ligation products, following manufacturer's instruction, and plated in LB AGAR+100 μg/ml Ampicillin over-night at 37° C.

Single-colonies of transformed E. coli bacteria are picked from LB AGAR plate and grown in 3 ml of LB medium+100 μg/ml Ampicillin (inoculation culture) over-night at 37° C. For maxiprep cultures, 0.5 ml of inoculation culture is grown in 250 ml of LB medium+100 μg/ml Ampicillin.

e. Purification of Plasmid DNA

Plasmid DNA is isolated from 250 ml of maxiprep culture of transformed E. coli bacteria by using PureLink HiPure Plasmid DNA Purification Kit (Invitrogen—K2100) applying manufacturer's instruction.

2. Selection of gRNAs Targeting β-Globin Gene: Design of Novel gRNAs

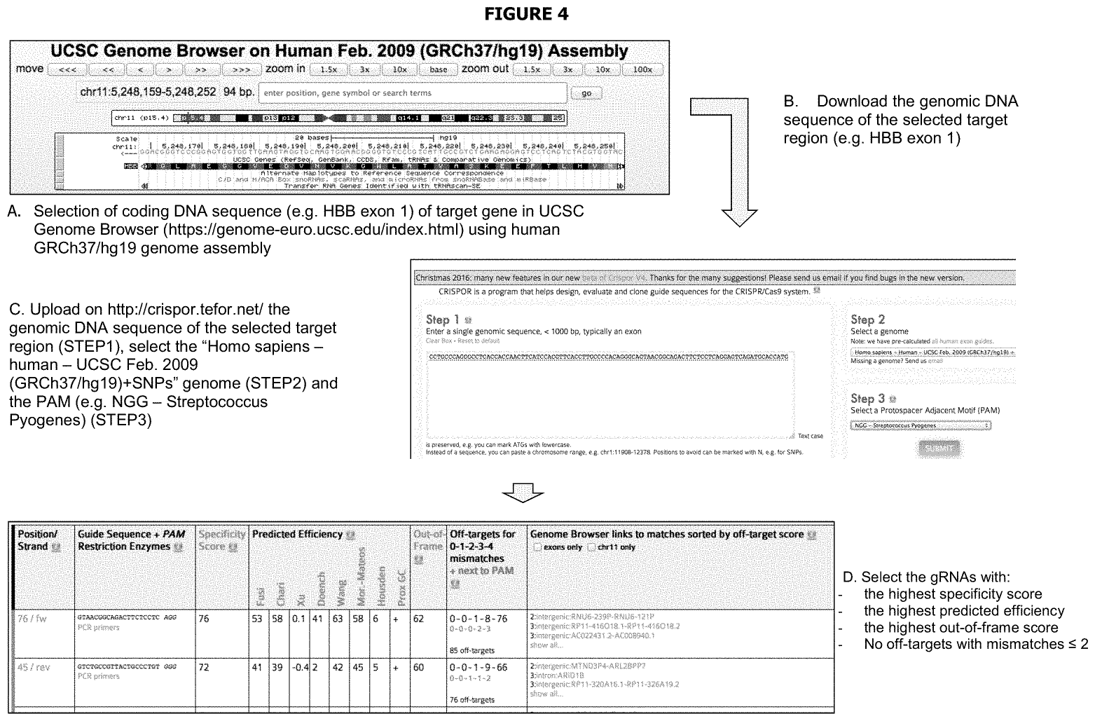

Novel gRNAs spacer-encoding sequences (F, G, H, I, J, K, L, M, N and O—respectively SEQ ID NOs: 27 to 36) were designed by using CRISPOR tool (http://crispor.tefor.net/). The genomic DNA sequence of the target region (e.g. exon 1 or exon 2 of HBB gene) was selected (FIG. 4A) using human GRCh37/hg19 genome assembly and downloaded (FIG. 4B) from UCSC Genome Browser (https://enone-euro.ucsc.edu/index.html). The genomic DNA sequence of the target region was uploaded on http://crispor.tefor.net/and gRNAs associated with a specific PAM (e.g. NGG—Streptococcus Pyogenes or NGA—S. Pyogenes mutant VQR) were designed based on the “Homo sapiens—human—UCSC February 2009 (GRCh37/hg19)+SNPs” genome (FIG. 4C). From the list of the resulting gRNAs, we selected the gRNAs with a highest (i) specificity score (cfdSpecScore ≥85), (ii) predicted efficiency (ChariEffScore ≥38) and (iii) out-of-frame score (≥60) and no off-targets with mismatches ≤2 in delta- and gamma-globin genes (FIG. 4D).

3. Cleavage Efficiency of gRNAs a, B, D and E in K562 and HUDEP-2 Erythroid Cell Lines

Fetal K562 and adult HUDEP-2 erythroid cells are known to naturally comprise the beta-globin gene in their genome. Therefore, we tested the gRNAs targeting the beta-globin gene in these cell lines.

One million cells were transfected with 4 μg of a Cas9-GFP expressing plasmid (pMJ920, Addgene plasmid #42234) and 0.8 μg of each gRNA-containing plasmid (MLM3636 gRNA A, MLM3636 gRNA B, MLM3636 gRNA D and MLM3636 gRNA E) in a 100 μl volume using Nucleofector I (Lonza). Control cells were treated with 4 μg of a Cas9-GFP expressing plasmid (pMJ920, Addgene plasmid #42234). We used AMAXA Cell Line Nucleofector Kit V (VCA-1003) for K562 and HUDEP-2 (T16 and L-29 programs). After transfection, K562 were maintained in RPMI 1640 medium (Lonza) containing 2 mM glutamine and supplemented with 10% fetal bovine serum (FBS, BioWhittaker, Lonza), HEPES (20 mM, LifeTechnologies), sodium pyruvate (1 mM, LifeTechnologies) and penicillin and streptomycin (100 U/ml each, LifeTechnologies) and HUDEP-2 were maintained as described in Canver et al., Nature, 2015, 527(7577):192-7. One week after transfection, DNA was extracted using PURE LINK Genomic DNA Mini kit (LifeTechnologies) following manufacturer's instructions.

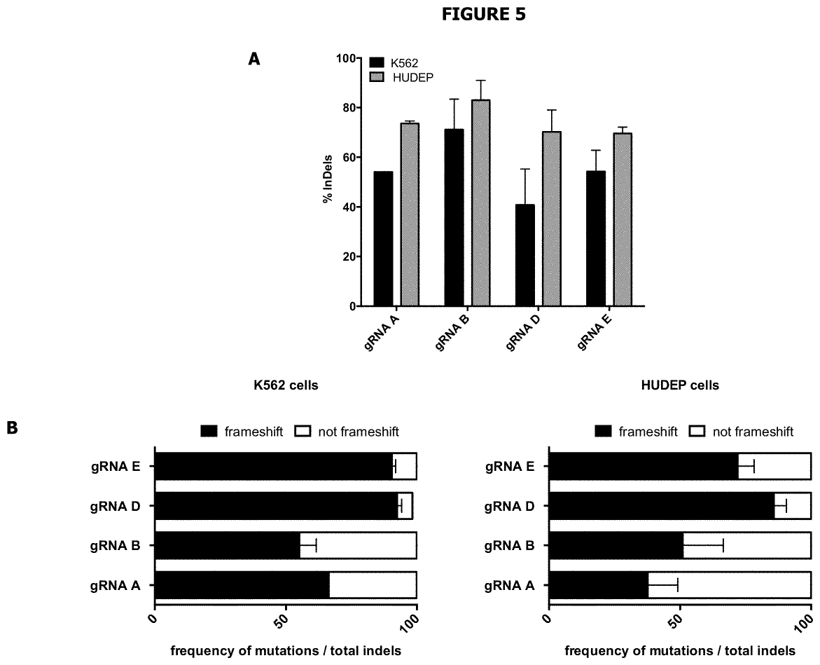

The genomic region of fetal K562 and adult HUDEP-2 erythroid cells encompassing the gRNA target sites was amplified by PCR. PCR was performed using primers HBBex1 F (5′-CAGCATCAGGAGTGGACAGA-3′, SEQ ID NO: 9) and HBBex1 R (5′-AGTCAGGGCAGAGCCATCTA-3′, SEQ ID NO: 10). We performed Sanger sequencing and TIDE analysis to evaluate the frequency of InDels and frameshift mutations. All the screened gRNAs (i.e. A, B, D, E) were able to cut at >35% of the genomic loci in transfected K562 and HUDEP-2 cells (FIG. 5A). The cells transfected with gRNA D led to the highest frequency of frameshift mutations, which resulted in the generation of stop-codons in the exon 1 (FIG. 5B). These results showed that gRNA A, B, D and E are particularly efficient to generate frameshift mutations of beta-globin gene in fetal K562 and adult HUDEP-2 erythroid cells resulting in the generation of stop codon in Exon 1.

### 4. Down-Regulation of Beta-Globin Expression in HUDEP-2

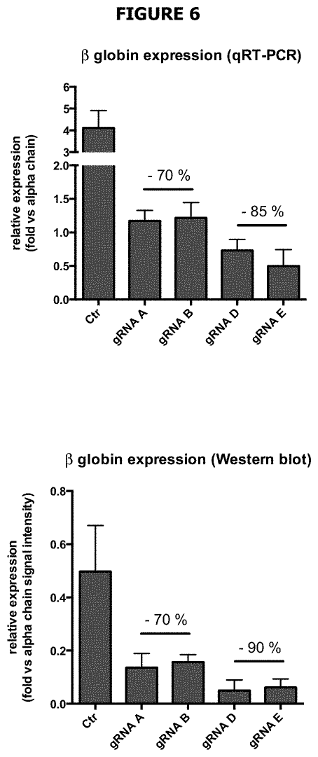

The efficiency of beta-globin knock-down was evaluated in HUDEP2 cells, which express high levels of the beta-globin chain (Kurita et al., PLoS One, 2013, 8(3):e59890). HUDEP-2 cells were transfected with 4 μg of a Cas9-GFP expressing plasmid (pMJ920, Addgene plasmid #42234) and 0.8 μg of each gRNA-containing plasmid (MLM3636 gRNA A, MLM3636 gRNA B, MLM3636 gRNA C and MLM3636 gRNA D), as described above (Example 3). Control cells were treated with 4 μg of a Cas9-GFP expressing plasmid (pMJ920, Addgene plasmid #42234). After one week, total RNA was extracted using RNeasy micro kit (QIAGEN) following manufacturer's instructions. Mature transcripts were reverse-transcribed using SuperScript First-Strand Synthesis System for RT-PCR (Invitrogen) with oligo(dT) primers. qRT-PCR was performed using SYBR green (Applied Biosystems). Primers HBB F (5′-GCAAGGTGAACGTGGATGAAGT-3′, SEQ ID NO: 11) and HBB R (5′-TAACAGCATCAGGAGTGGACAGA-3′, SEQ ID NO: 12) were used to amplify the beta-globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) were used to amplify the alpha-globin transcripts. Beta-globin expression results were normalized to alpha-globin. In parallel, total proteins were extracted in lysis buffer [PBS 1×, 50 mM, TriS-HCl PH 7.4-7.5, 150 mM NaCl, 0,5% DOC, 0,1% SDS, 2 mM EDTA, 1% Triton, protease inhibitor 7× (EDTA-Free Protease Inhibitor Cocktail, Roche) and phosphatase inhibitor 10× (PhosphoSTOP, Roche)], subjected to 3 rounds of sonication (three cycles of 10 pulses, Amplitude 0.7, 0.5 s oscillation) and to 3 freeze/thaw cycles (3 min each). Lysates were centrifuged at 12.000×g for 12 min at 4° C., and supernatants were used for western blot analysis. We measured protein content using the Bradford Protein Assay kit with bovine serum albumin (BSA) as reference standard. After boiling for 5 min in loading buffer (30% glycerol, 5% SDS, 9.25% Dithiothreitol, 1 μl of Bromophenol Blue, Tris-HCl 0.5 M, pH 6.8). samples containing 20-50 μg protein were separated using a 15% acrylamide gel SDS-PAGE electrophoresis. The transfer was performed at 250 mA for 2 hour at 4° C. or room temperature (RT). The PDVF membranes were dried and then incubated in blocking solution TBS-Tween 0.1% (Tris-Buffered Saline+Tween 20; TBS-T; Sigma Aldrich) 5% milk over-night at 4° C., and stained for 1-2 hours at RT with primary antibodies diluted in TBS-Tween 5% milk solution. The primary antibodies are specific for beta-globin (dilution 1:200; hemoglobin beta (37-8), sc-21757, Santa Cruz Biotechnology) and alpha-globin (dilution 1:200; hemoglobin alpha (D-16), sc-31110, Santa Cruz Biotechnology). After 3 washes (10 minutes each) in TBS-Tween, antibody staining was revealed using HRP-conjugated anti-mouse (1:5.000; Thermo Scientific) and HRP-conjugated anti-goat (1:5.000; Thermo Scientific) for 1 hour at RT in TBS-T 5% milk solution. Blots were developed with ECL system (Immobilon Western, Millipore) and were exposed to x-ray films (different exposure times according to the intensity of signals). Membranes were stripped for 15′ with Stripping Buffer (Thermo Scientific). The bands corresponding to beta-globin were quantified by using ImageJ software and/or Gel Pro software and the values (in pixels) obtained were normalized to those of the alpha-globin bands. Both qRT-PCR (FIG. 6) and Western Blot (FIG. 6) analysis showed a reduction in the beta-globin expression in cells treated with Cas9+gRNAs targeting HBB gene, which was more pronounced in cells electroporated in the presence of the gRNAs allowing the highest frequency of frameshift mutations (gRNA D and E).

These results showed that gRNA A, B, D and E are particularly efficient to disrupt the expression of beta-globin in HUDEP-2 cells.

5. Cleavage Efficiency of Selected gRNAs in HSPCs
5.1 Transfection of Primary HSPCs with gRNA B, D and E: Editing Efficiency

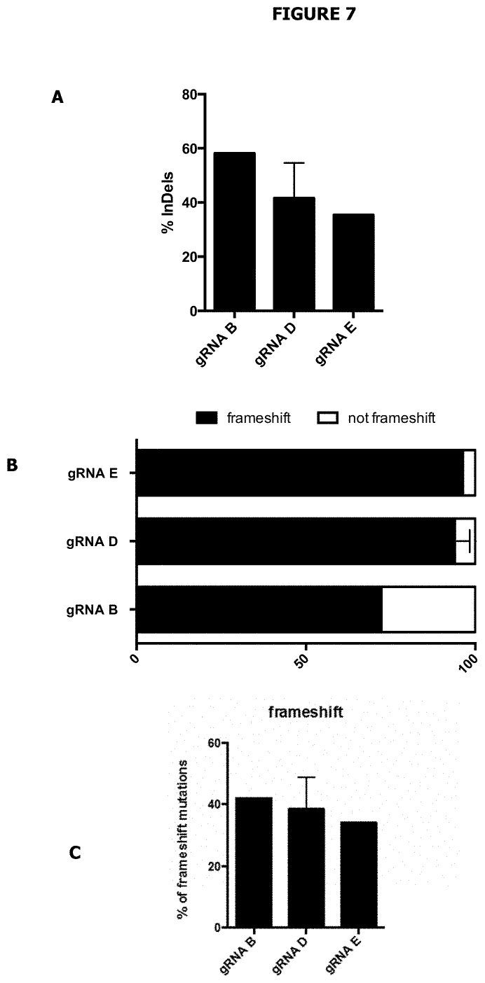

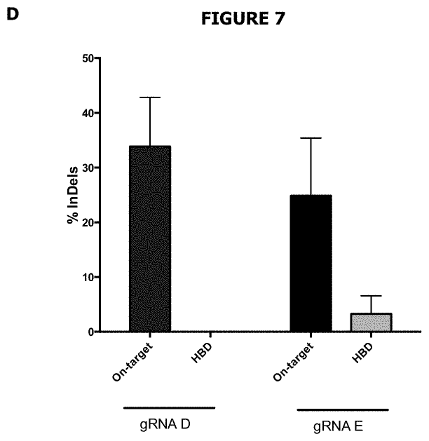

gRNAs allowing the highest frequency of frameshift mutations (B, D and E) were tested in adult HSPC from a healthy donor. HSPC were cultured in expansion medium: StemSpan SFEM medium (StemCell Technologies), containing 2 mM glutamine, penicillin and streptomycin (100 U/ml each, Gibco, LifeTechnologies), Flt3-Ligand (300 ng/ml, Peprotech), SCF (300 ng/ml, Peprotech), TPO (100 ng/ml, Peprotech) and IL3 (60 ng/ml, Peprotech). 48 hours after thawing, one million cells were transfected with 4 μg of a Cas9-GFP expressing plasmid (pMJ920, Addgene plasmid #42234) and 1.6 μl of each gRNA-containing plasmid (MLM3636 gRNA B, MLM3636 gRNA C and MLM3636 gRNA D) in a 100 μl volume using Nucleofector I (Lonza). Control cells were treated with 4 μg of a Cas9-GFP expressing plasmid (pMJ920, Addgene plasmid #42234). We used AMAXA Human CD34 Cell Nucleofector Kit (VPA-1003) for HSPC (U-08 program). After transfection, HSPC were maintained in the same medium supplemented with Z-VAD-FMK (120 uM, InvivoGen) and StemRegenin 1 (750 uM, Stem Cell Technologies). On day 5 after transfection, DNA was extracted to evaluate the editing efficiency, as described above for K562 and HUDEP-2 cells (Example 3). Genome editing efficiency was higher for gRNA B (FIG. 7A), however the rate of frameshift mutations generated by gRNA B was lower compared to gRNA D and E (FIG. 7B). Overall, gRNA B and D allowed the highest absolute frequency of frameshift mutations (FIG. 7C) in HSPC. However, gRNA D was selected for the following experiments, because it generated non-frameshift mutations at a lower frequency (FIG. 7B) and did not have predicted off-targets in the beta-like globin genes.

These results showed that gRNA B, D and E are particularly efficient to generate frameshift mutations of beta-globin gene in HSPC.

### 5.2 Transfection of Primary HSPC Cells: Off Target Analysis

To evaluate off-target activity in primary HSPCs, plasmids encoding the selected gRNAs were individually delivered together with a Cas9-GFP-expressing plasmid to cord blood-derived CD34+ HSPCs. Protocol is slightly different from 5.1. Cells were transfected with 4 μg of Cas9-GFP expressing plasmid and 3.2 μg of each gRNA-containing vector using Nucleofector I (Lonza), AMAXA Human CD34 Cell Nucleofector Kit (VPA-1003) and U08 program. Transfection efficiency was verified by flow cytometry analyses 18 hours after electroporation (30-50% of GFP+ Cas9-expressing cells).

TIDE (Tracking of Indels by Decomposition) analysis (Brinkman E K et al., 2014) of the genomic region containing HBB exon 1 and amplified from genomic DNA extracted 4 days after transfection showed that gRNA D and E display a cleavage efficiency of ≈35% and ≈25%, respectively, with a frequency of frameshift mutations of 90-95% for both the gRNAs (not shown). Conversely, gRNA B displays an editing efficiency of ≈60% with a lower frequency of frameshift mutations in comparison with gRNA D and E (not shown). TIDE analysis the genomic region containing HBD exon 1 showed absence of InDels in samples treated with gRNA D, whereas ≈3% of HBD alleles are edited (“off-target”) upon treatment with gRNA E (FIG. 7D). This result can be explained by the low number of mismatches (2) between gRNA E sequence and the corresponding off-target in HBD exon 1 (FIG. 3), whereas a higher number of mismatches is observed for gRNA D (4; FIG. 3), which likely decreases the probability of off-target activity in the HBD gene.

### 6. Down-Regulation of Beta-Globin Expression in HSPC-Derived Erythroid Cells

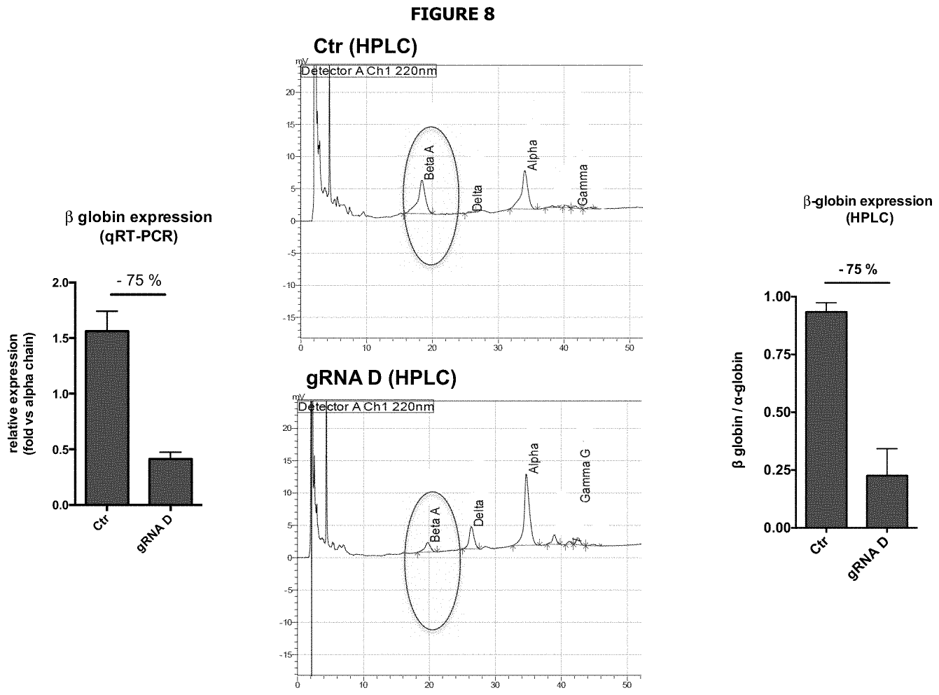

Cas9 and gRNA D were delivered by plasmid transfection in adult HSPC derived from a healthy donor (plasmids pMJ920 Cas9-GFP and MLM3636 gRNA D) as described above (Example 5). Control cells were electroporated in the presence of the plasmid pMJ920. One day after, GFP-positive HSPC were sorted by FACS 2 days after transfection, HSPC were differentiated towards the erythroid lineage in liquid culture as previously described (Sankaran, Science, 2008, 322(5909):1839-42). After 11 days, RNA was extracted from mature erythroid cells to evaluate the beta-globin expression levels. Total RNA was extracted using RNeasy micro kit (QIAGEN) following manufacturer's instructions. Mature transcripts were reverse-transcribed using SuperScript First-Strand Synthesis System for RT-PCR (Invitrogen) with oligo(dT) primers. qRT-PCR was performed using SYBR green (Applied Biosystems). Primers HBB F (5′-GCAAGGTGAACGTGGATGAAGT-3′, SEQ ID NO: 11) and HBB R (5′-TAACAGCATCAGGAGTGGACAGA-3′, SEQ ID NO: 12) were used to amplify the beta-globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) were used to amplify the alpha-globin transcripts. Beta-globin expression results were normalized to alpha-globin. In parallel, reverse phase HPLC (RP-HPLC) analysis of globin chains was performed using a NexeraX2 SIL-30AC chromatograph (Shimadzu) and the LC Solution software. Globin chains from in vitro differentiated mature erythroblasts were separated by HPLC using a 250×4.6 mm, 3.6 μm Aeris Widepore column (Phenomenex). Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm. Both qRT-PCR and RP-HPLC analyses showed a dramatic down-regulation of beta-globin expression in mature erythroblasts electroporated with plasmid MLM3636 gRNA D (FIG. 8).

These results showed that gRNA D is particularly efficient to disrupt the expression of beta-globin in HSPC-derived erythroblasts.

### Example 4: Optimization of gRNA Activity

The original gRNA scaffold developed by Cong et al., Science, 2013, 339(6121):819-23 was recently optimized by Dang et al., Genome Biol, 2015, 16:280 to increase knock-out efficiency.

The gRNA spacer-encoding sequences B, D and E (respectively SEQ ID NOs: 24, 25 and 26) were cloned in Dang p.hU6 gRNA plasmids (Addgene #53188), generating the following plasmids:
Dang p.hU6 gRNA B coding for gRNA B
Dang p.hU6 gRNA D coding for gRNA D
Dang p.hU6 gRNA E coding for gRNA E

For the generation of Dang p.hU6 plasmids (Addgene #53188) carrying gRNA B, D and E, the following protocol was applied:

a. Annealing gRNA Oligos

### Oligonucleotide Sequences:

| | | SEQ |
|---|---|---|
| | | ID |
| Oligo Name | Sequence 5′ to 3′ (*) | No: |
| Oligo FOR-Opt_ | CACCGTAACGGCAGACTTCTCCTC | 15 |
| gRNA B | | |
| Oligo REV-Opt_ | AAACGAGGAGAAGTCTGCCGTTAC | 16 |
| gRNA B | | |
| Oligo FOR-Opt_ | CACCGTCTGCCGTTACTGCCCTGT | 17 |
| gRNA D | | |
| Oligo REV-Opt_ | AAACACAGGGCAGTAACGGCAGAC | 18 |
| gRNA D | | |
| Oligo FOR-Opt_ | CACCGAAGGTGAACGTGGATGAAGT | 19 |
| gRNA E | | |
| Oligo REV-Opt_ | AAACACTTCATCCACGTTCACCTTC | 20 |
| gRNA E | | |

(*)In bold: nucleotide sequence encoding the gRNA spacer

Preparation of MIX 1 for gRNA oligo annealing [8 μl 10 μM gRNA oligo FOR-Opt, 8 μl 10 μM gRNA oligo REV-Opt, 2 μl 10×NEB Ligase buffer (Biolabs—M22OO), 2 μl DEPC-water]. Annealing reaction in PCR machine, following this PCR program: from 96° C. 300 seconds, 85° C. 20 seconds, 75° C. 20 seconds, 65° C. 20 seconds, 55° C. 20 seconds, 45° C. 20 seconds, 35° C. 20 seconds, 25° C. 20 seconds

b. Digestion of Dang p.hU6 Plasmid

Incubate the digestion mix reaction [x μl (20 μg) of Dang p.hU6 plasmid (Addgene #53188), 10 μl of BbsI enzyme (100 U), 10 μl of enzyme buffer 10×, (100−x) μl of DEPC-water] over-night at 37° C. Purify from low melting agarose (0.8%) gel the linearized Dang p.hU6 plasmid (size: 3515 bp) with QIAquick Gel Extraction Kit (QIAGEN).

c. Insertion of gRNA within Dang p.hU6 Plasmid

Incubation of ligation mix [x μl (50 ng) linearized MA128.hU6 plasmid, 1 μl of annealed gRNA oligos, 1 μl of 10× Ligase Buffer, 1 μl of Ligase (QUICK LIGASE NEB—M2200), (10−x) μl of DEPC-water] for 15 minutes at room temperature.

d. Transformation of Bacteria and Amplification of Plasmid

Chemical competent E. coli bacteria (One Shot TOP10 Chemically competent E. Coli—Invitrogen—C4040) are transformed with 5 μl of ligation products, following manufacturer's instruction, and plated in LB AGAR+100 μg/ml Ampicillin over-night at 37° C.

Single-colonies of transformed E. coli bacteria are picked from LB AGAR plate and grown in 3 ml of LB medium+100 μg/ml Ampicillin (inoculation culture) over-night at 37° C. For maxiprep cultures, 0.5 ml of inoculation culture is grown in 250 ml of LB medium+100 μg/ml Ampicillin.

e. Purification of Plasmid DNA

Plasmid DNA is isolated from 250 ml of maxiprep culture of transformed E. coli bacteria by using PureLink HiPure Plasmid DNA Purification Kit (Invitrogen—K2100) applying manufacturer's instruction.

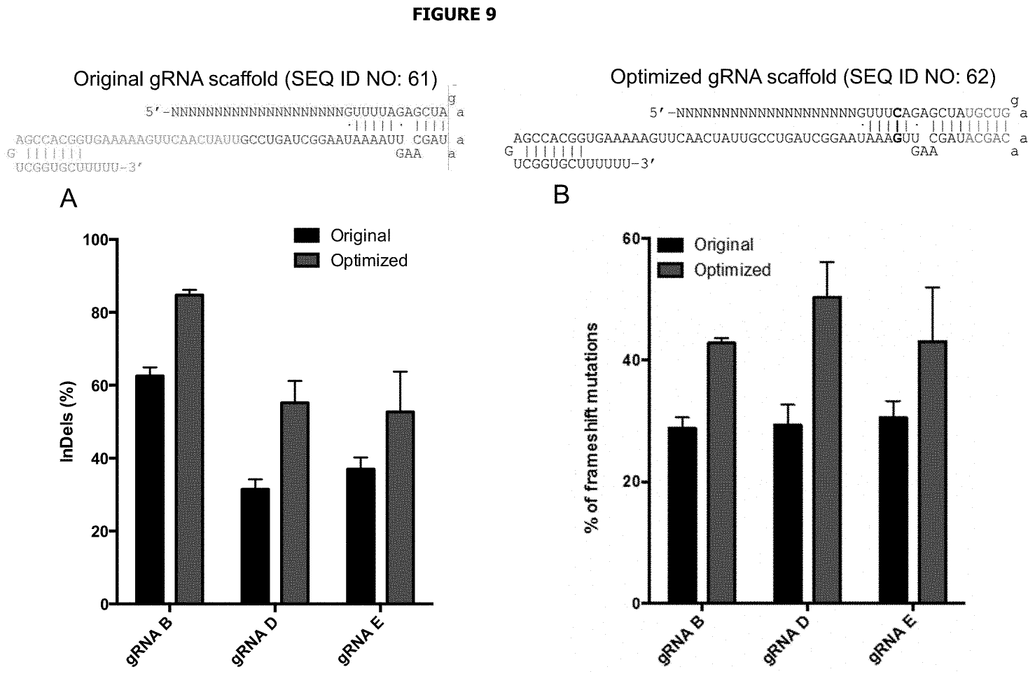

One million of K562 cells were transfected with 4 μg of a Cas9-GFP expressing plasmid (pMJ920, Addgene plasmid #42234) and 0.8 of each gRNA-containing plasmid (MLM3636 gRNA B, MLM3636 gRNA C and MLM3636 gRNA D, Dang p.hU6 gRNA B, Dang p.hU6 gRNA C and Dang p.hU6 gRNA D) in a 100 μl volume using Nucleofector I (Lonza). Control cells were treated with 4 μg of a Cas9-GFP expressing plasmid (pMJ920, Addgene plasmid #42234). We used AMAXA Cell Line Nucleofector Kit V (VCA-1003) for K562 cells (T16 program). After transfection, K562 were maintained in RPMI 1640 medium (Lonza) containing 2 mM glutamine and supplemented with 10% fetal bovine serum (FBS, BioWhittaker, Lonza), HEPES (20 mM, LifeTechnologies), sodium pyruvate (1 mM, LifeTechnologies) and penicillin and streptomycin (100 U/ml each, LifeTechnologies). One week after transfection, DNA was extracted using PURE LINK Genomic DNA Mini kit (LifeTechnologies) following manufacturer's instructions. All the gRNAs with the optimized structure (Dang p.hU6 gRNA B, Dang p.hU6 gRNA C and Dang p.hU6 gRNA D; Dang et al., Genome Biol, 2015, 16:280) show higher InDels efficiency (FIG. 9A) and frequency of frameshift mutation in HBB gene (FIG. 9B) compared to the corresponding gRNAs with original structure (MLM3636 gRNA B, MLM3636 gRNA C and MLM3636 gRNA D; Cong et al., Science, 2013, 339(6121):819-23).

These results showed that the modification of the scaffold in the gRNAs targeting the beta-globin gene (see Example 3) can further increase their frequency of gene disruption.

### Example 5: Construction of a Recombinant Viral Vector (i.e. Lentivector) According to the Invention

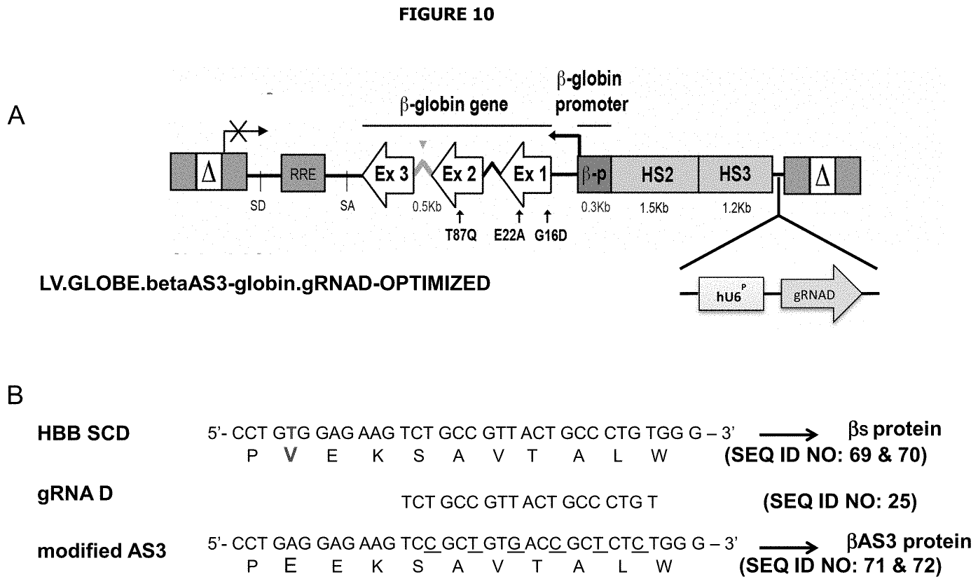

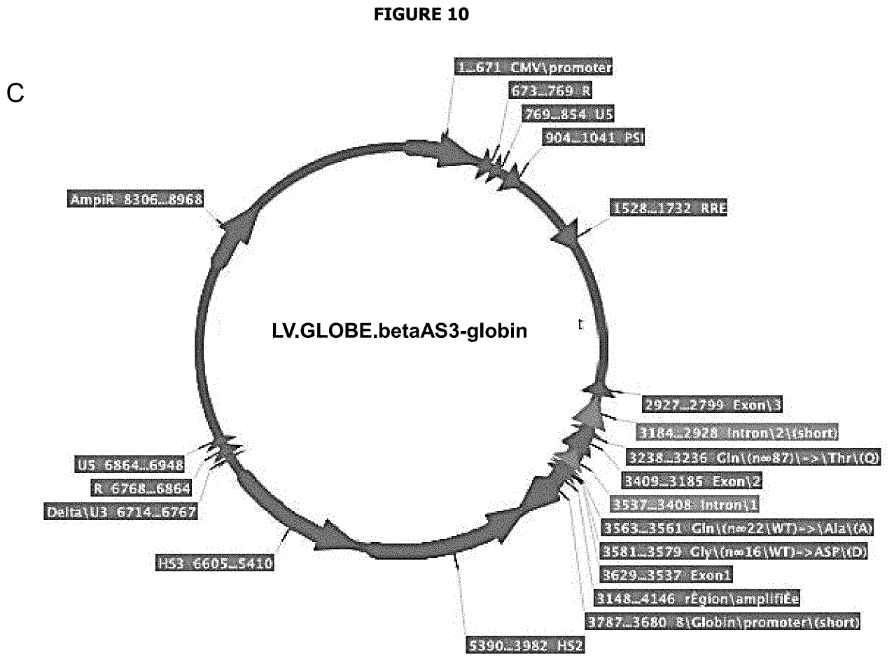

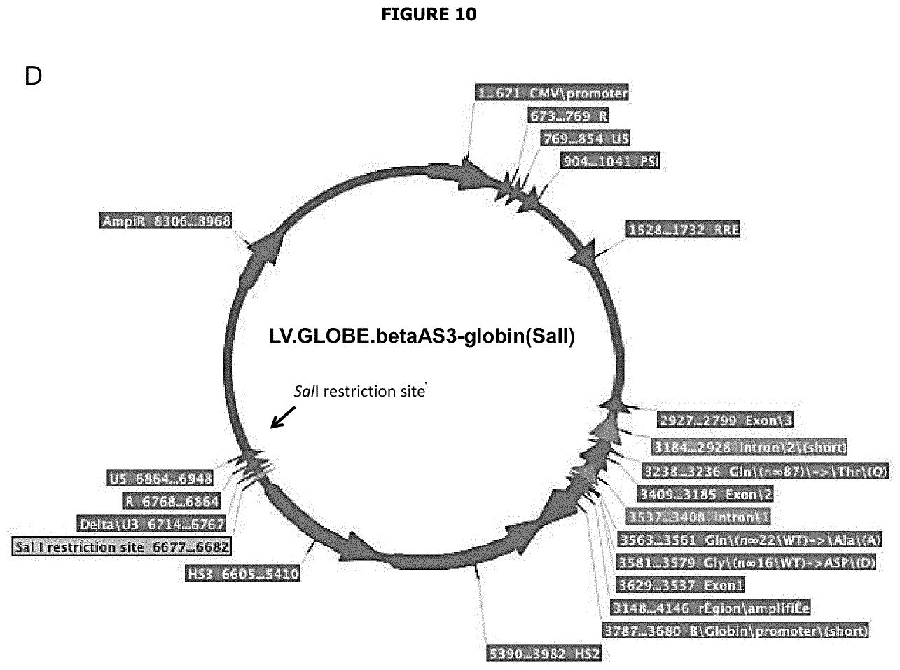

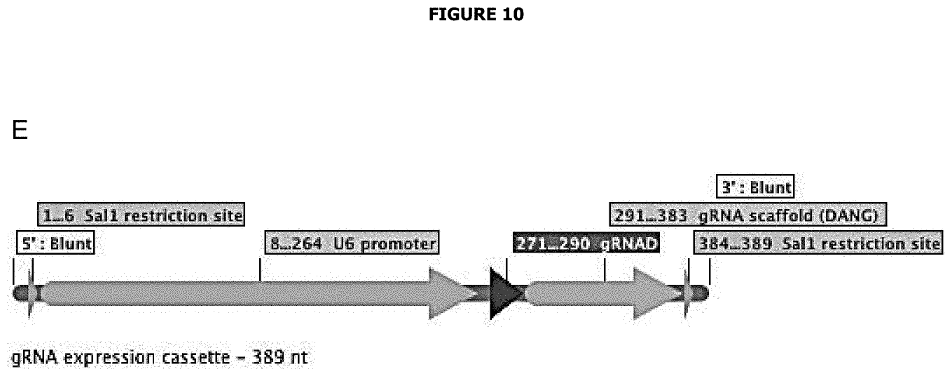

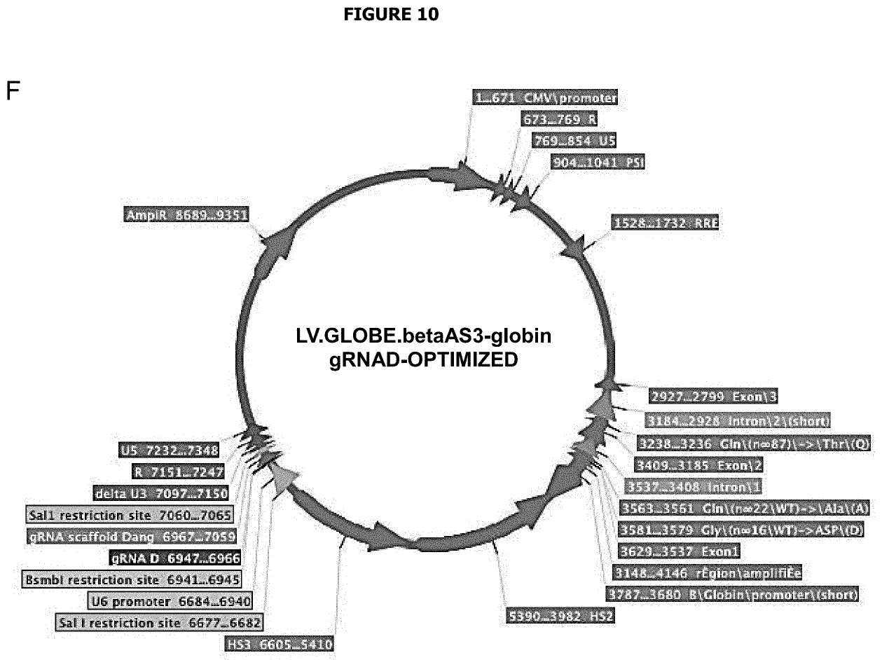

The LV.GLOBE.betaAS3-globin.gRNA D-OPTIMIZED lentiviral construct (FIG. 10A, such as SEQ ID NO: 47) carries: (1) an anti-sickling gene (FIG. 10B, e.g. modified Beta AS3 SEQ ID NO: 8) harboring silent mutations (indicated as underscored letters in FIG. 10B) inserted by site-directed mutagenesis in order to impair the gRNA binding to the transgene and the three antisickling mutations [Gly16Asp (G16D), Glu22Ala (E22A) and Thr87Gln (T87Q)] in the exons 1 and 2 (FIG. 10A); (2) a gRNA showing (i) a high efficiency of beta-globin gene disruption; (ii) a high rate of frameshift mutations; (iii) a low off-target activity (e.g. no off-targets in the beta like-globin genes), such as gRNA D (FIG. 10B), under the control of the human U6 promoter (FIG. 10A).

In FIGS. 10C, 10D, 10E and 10F, (A.) the restriction site SalI is inserted between HS3 and DeltaU3 elements of the LV.GLOBE.betaAS3-globin plasmid (FIG. 10C; SEQ ID NO: 45) by site-directed mutagenesis to generate the LV.GLOBE. betaAS3-globin (SalI) plasmid (SEQ ID NO: 46). (B.) A DNA fragment containing the hU6 promoter and the gRNA-encoding sequence (e.g. gRNA D) flanked by SalI restriction sites (called “gRNA expression cassette”; FIG. 10E) is synthesized. (C.) LV.GLOBE. betaAS3-globin (SalI) plasmid (SEQ ID NO: 46) is digested [digestion mix reaction: x μl (20 μg) of LV.GLOBE. betaAS3-globin (SalI) plasmid (SEQ ID NO: 46), 10 μl of SalI enzyme (100 U), 10 μl of enzyme buffer 10×, (100−x) μl of DEPC-water] over-night at 37° C. The linearized LV.GLOBE. betaAS3-globin-globin(SalI) plasmid (size: 10195 bp) is purified by low melting agarose (0.8%) gel using QIAquick Gel Extraction Kit (QIAGEN). In parallel, the gRNA expression cassette is digested [digestion mix reaction: x μl (20 μg) of gRNA expression cassette, 10 μl of SalI enzyme (100 U), 10 μl of enzyme buffer 10×, (100−x) μl of DEPC-water] over-night at 37° C. The linearized gRNA expression cassette (size: 383 bp) is purified by low melting agarose (1.5%) gel using QIAquick Gel Extraction Kit (QIAGEN). (D.) The gRNA expression cassette is inserted within LV.GLOBE. betaAS3-globin—globin(SalI) plasmid through incubation of ligation mix [x μl (50 ng) linearized gRNA expression cassette, y μl (50 ng) linearized LV.GLOBE. betaAS3-globin-globin(SalI) plasmid, 1 μl of 10× Ligase Buffer, 1 μl of Ligase (QUICK LIGASE NEB—M2200), (10−x−y) μl of DEPC-water] for 15 minutes at room temperature. Chemical competent E. coli bacteria (One Shot TOP10 Chemically competent E. Coli—Invitrogen—C4040) are transformed with 5 μl of ligation products, following manufacturer's instruction, and plated in LB AGAR+100 μg/ml Ampicillin over-night at 32° C. Single-colonies of transformed E. coli bacteria are picked from LB AGAR plate and grown in 50 ml of LB medium+100 μg/ml Ampicillin (miniprep cultures) over-night at 32° C. Plasmid DNA is isolated from 10 ml of miniprep culture of transformed E. coli bacteria by using PureLink HiPure Plasmid DNA Purification Kit (Invitrogen—K2100) applying manufacturer's instruction. Plasmid DNA will be analyse by Sanger-sequencing to verify that gRNA expression cassette is inserted in the opposite orientation compare to betaAS3-globin expression cassette. Miniprep cultures (10 ml) derived from colonies containing plasmids fitting these criteria are grown in 250 ml of LB medium+100 μg/ml Ampicillin over-night at 32° C. Plasmid DNA is isolated from 250 ml of maxiprep culture of transformed E. coli bacteria by using PureLink HiPure Plasmid DNA Purification Kit (Invitrogen—K2100) applying manufacturer's instruction. The isolated plasmid DNA (LV.GLOBE.betaAS3-globin.gRNA D-OPTIMIZED; FIG. 10F, SEQ ID NO: 47) is used as backbone for recombinant lentiviral vector production.

### Example 6: Transduction of HSPC with a Recombinant Lentiviral Vector According to the Invention and Introduction of Cas9 into the Transduced Cell

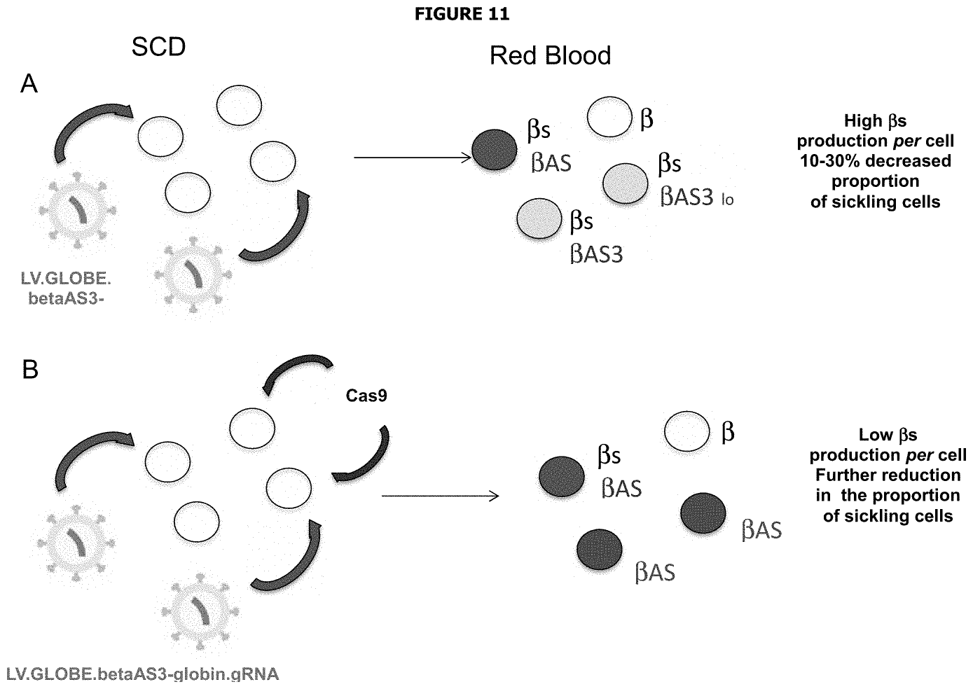

(A) In the classical gene therapy approach the lentiviral vector expressing an anti-sickling gene (e.g. LV.GLOBE.beta-globin and LV.AS3 (Romero et al., JCI, 2016)) does not strongly reduce the sickle beta-globin expression in the erythroid progeny of SCD HSPC and allows the correction of only 10% to 30% of mature Red Blood Cells (FIG. 11A).

(B) SCD HSPC are transduced with the gamma-beta hybrid globin and gRNA expressing lentiviral vector (e.g. LV.GLOBE.gamma-beta-globin.gRNA) and Cas9 is delivered transiently. This approach allows the expression of an anti-sickling transgene and the concomitant reduction of the sickle beta-globin levels, which will lead to an increase frequency of corrected Red Blood Cells Importantly, Cas9-mediated disruption of the sickle beta-globin gene will be observed only in transduced SCD cells where the knock out of the sickle beta-globin is compensated by the expression of the anti-sickling gene, thus avoiding an absence of Beta like chain leading to the risk of alpha-chain precipitation, leading to cell death and anemia, as observed in beta-thalassemia (FIG. 11B).

### Example 7: Genetic Modification of Patient SCD HSPC In Vitro

SCD CD34⁺ HSPC are transduced with lentiviral vectors expressing an anti-sickling gene and a gRNA targeting the beta-globin gene (e.g. LV.GLOBE.betaAS3-globin.gRNAD-OPTIMIZED, SEQ ID NO: 47 or LV.GLOBE-AS3modified.gRNAD, SEQ ID NO: 94) or the intronic erythroid-specific BCL11A enhancer (e.g. LV.GLOBE-AS3modified.gRNA-BCL11Aenhancer, SEQ ID NO: 75) or the gamma-globin promoters (e.g. LV.GLOBE-AS3modified.gRNA-13 bp-del, SEQ ID NO: 76) and Cas9 is delivered transiently (DNA-, RNA-, protein- or lentiviral-delivery).

HSPC derived from bone marrow or mobilized peripheral blood of SCD patients are cultured in RetroNectin (20 μg/ml, Takara Shuzo Co.)-coated plates in expansion medium (pre-activation step): StemSpan SFEM medium (StemCell Technologies), containing 2 mM glutamine, penicillin and streptomycin (100 U/ml each, Gibco, LifeTechnologies), Flt3-Ligand (300 ng/ml, Peprotech), SCF (300 ng/ml, Peprotech), TPO (100 ng/ml, Peprotech) and IL3 (60 ng/ml, Peprotech). 24 hours after thawing (day 1), 200.000 cells are transduced with LV.GLOBE.betaAS3-globin.gRNAD-OPTIMIZED (SEQ ID NO: 47) (MOI 20-100) in expansion medium+protein sulfate (4 μg/ml) and plated in RetroNectin (20 μg/ml, Takara Shuzo Co.)-coated 96-well plates. Control cells are transduced with LV.GLOBE. betaAS3-globin. (SalI) (SEQ ID NO: 46) (MOI 20-100) or LV.GLOBE.gRNAD (MOI 20-100) (LV.GLOBE vector carrying gRNA expression cassette without beta AS3 globin transgene). Medium is change 24 hours after transduction (day 2) and 1-3*10⁶ cells are transfected with 20 μg of Cas9 mRNA modified with pseudouridine and 5-methylcytidine to reduce immune stimulation (Trilink, #L-6125) in a 100 μl volume using Nucleofector 4D (Lonza). Alternatively, 1-3*10⁵ cells are transfected with 30-180 Cas9 pmol in a 20 μl volume using Nucleofector 4D (Lonza). We use AMAXA Human CD34 Cell Nucleofector Kit (VPA-1003) for HSPC (CA137 program). After transfection, HSPC were maintained in the same medium supplemented with Z-VAD-FMK (120 uM, InvivoGen) and StemRegenin 1 (750 uM, Stem Cell Technologies). The day after (day 3), treated HSPC are either in vitro differentiated towards the erythroid lineage using a 3-phase liquid erythroid culture system (Giarratana et al., Blood, 2011, 118(19):5071-9) or plated in a semi-solid medium containing cytokines supporting the growth of erythroid and myeloid hematopoietic progenitors (Clonal progenitor assay; medium GFH4435, Stem Cell Technologies). On day 13 of liquid culture and clonal progenitor assay, samples are collected for DNA extraction to evaluate the editing efficiency, as described above for K562 and HUDEP-2 cells (example 3), and the frequency of transduced cells in bulk (erythroid) and clonal culture by PCR followed by Tracking of In/Dels by Decomposition (Brinkman E K, Chen T, Amendola M, and van Steensel B. Easy quantitative assessment of genome editing by sequence trace decomposition. Nucleic acids research. 2014; 42(22):e168.) also called TIDE analysis (as described in example 3) and qPCR (using primers recognizing specifically the lentiviral vector; Miccio et al., Proc Natl Acad Sci USA, 2008, 105(30):10547-52), respectively.

A genome-wide analysis of Double Strand Breaks using Genome-wide, unbiased identification of DSBs enabled by sequencing, also called GUIDE-seq (Tsai et al., Nat Biotechnol, 2015, 33(2):187-97) is performed to detect and quantify off-target cleavage sites in HSPC and their differentiated progeny (DNA extracted from samples collected at day 13 of clonal progenitor assay). LV integration sites in SCD HSPC are analyzed in order to evaluate the potential genotoxic risk of globin-expressing LV vectors. Integration sites are amplified by ligation-mediated PCR, sequenced and mapped to the human genome, as previously described (Romano et al., Sci Rep, 2016, 6:24724). The anti-sickling globin and betaS-globin expression are evaluated by qRT-PCR in samples collected upon 13, 16, 18 and 21 days of liquid culture differentiation. Total RNA is extracted using RNeasy micro kit (QIAGEN) following manufacturer's instructions. Mature transcripts are reverse-transcribed using SuperScript First-Strand Synthesis System for RT-PCR (Invitrogen) with oligo(dT) primers. qRT-PCR was performed using SYBR green (Applied Biosystems). Primers HBB F (5′-GCAAGGTGAACGTGGATGAAGT-3′, SEQ ID NO: 11) and HBB R (5′-TAACAGCATCAGGAGTGGACAGA-3′, SEQ ID NO: 12) are used to amplify the beta-globin transcripts and primers HBB-AS3 F (5′-AAGGGCACCTTTGCCCAG-3′, SEQ ID NO: 21) and HBB-AS3 R (5′-GCCACCACTTTCTGATAGGCAG-3′, SEQ ID NO: 22) are used to amplify the beta AS3 globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) are used to amplify the alpha-globin transcripts. Beta-globin expression results are normalized to alpha-globin. In parallel, reverse phase HPLC (RP-HPLC) analysis is performed (as described above in Example 6) in genetically modified HSPC differentiated in vitro into fully mature, enucleated Red Blood Cells (day 21 of liquid culture differentiation). The recovery of functional RBC properties is assessed enucleated Red Blood Cells (day 21 of liquid culture differentiation) by evaluating the reversion of the sickling and the correction of the increased adhesiveness and rigidity of SCD cells, features involved in the pathological occurrence of vaso-occlusive events (Picot et al., Am J Hematol, 2015, 90(4):339-45). Sickling dynamics is evaluated in enucleated Red Blood Cells (day 21 of liquid culture differentiation) exposing the cells to an oxygen-deprived atmosphere (0% O₂). Time-course of sickling is monitored in real-time by video microscopy for 1 hour, capturing images every 5 minutes using the AxioObserver Z1 microscope (Zeiss) and a 40× objective.

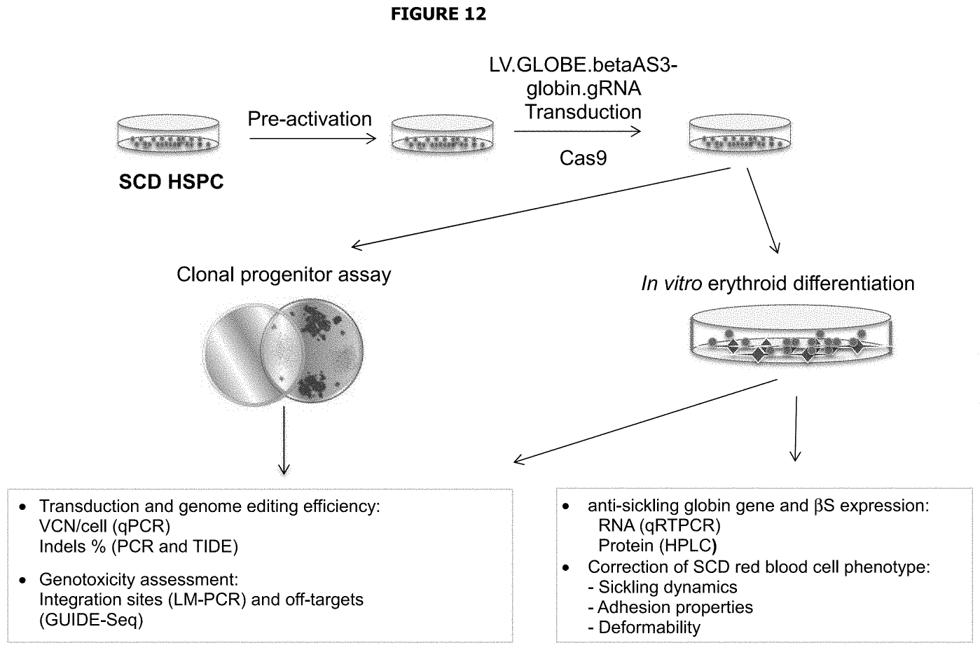

This process is illustrated in FIG. 12.

Such method is applied mutatis mutandis when using any of lentiral vectors of the invention.

### Example 8: Genetic Modification of Patient SCD HSC In Vivo

The engraftment capability of genetically modified patient SCD HSC and the efficacy of the therapeutic approach in Red Blood Cells derived from engrafting SCD HSC are assessed in in vivo mouse experiments. The in vivo frequency of modified HSC and the efficacy of the therapeutic strategy have to be similar to the same parameters measured in vitro in HSPC to exclude any HSC impairment due to our treatment.

HSPC derived from bone marrow or mobilized peripheral blood of SCD patients are cultured in RetroNectin (20 μg/ml, Takara Shuzo Co.)-coated plates in expansion medium (pre-activation step): StemSpan SFEM medium (StemCell Technologies), containing 2 mM glutamine, penicillin and streptomycin (100 U/ml each, Gibco, LifeTechnologies), Flt3-Ligand (300 ng/ml, Peprotech), SCF (300 ng/ml, Peprotech), TPO (100 ng/ml, Peprotech) and IL3 (60 ng/ml, Peprotech). 24 hours after thawing (day 1), 1-2*10⁶ cells are transduced with a lentiviral vector expressing an anti-sickling gene and a gRNA targeting the beta-globin gene (e.g. LV.GLOBE.betaAS3-globin.gRNAD-OPTIMIZED, SEQ ID NO: 47 or LV.GLOBE-AS3modified.gRNAD, SEQ ID NO: 94) or a gRNA targeting the intronic erythroid-specific BCL11A enhancer (LV.GLOBE-AS3modified.gRNA-BCL11Aenhancer, SEQ ID NO: 75) or a gRNA targeting the gamma-globin promoters (LV.GLOBE-AS3modified.gRNA-13 bp-del, SEQ ID NO: 76) (MOI 20-100) in expansion medium+protein sulfate (4 μg/ml) and plated in RetroNectin (20 μg/ml, Takara Shuzo Co.)-coated 96-well plates. Control cells are transduced with LV.GLOBE.gamma-beta-globin(SalI) (MOI 20-100) and LV.GLOBE.gRNAD (MOI 20-100) (LV.GLOBE vector carrying gRNA expression cassette without beta AS3 globin transgene). Medium is change 24 hours after transduction (day 2) and 1-3*10⁶ cells are transfected with 20 μg of Cas9 mRNA modified with pseudouridine and 5-methylcytidine to reduce immune stimulation (Trilink, #L-6125) in a 100 μl volume using Nucleofector 4D (Lonza). Alternatively, 1-3*10⁵ cells are transfected with 30-180 Cas9 pmol in a 20 μl volume using Nucleofector 4D (Lonza). We use AMAXA Human CD34 Cell Nucleofector Kit (VPA-1003) for HSPC (CA137 program). After transfection, HSPC were maintained in the same medium supplemented with Z-VAD-FMK (120 uM, InvivoGen) and StemRegenin 1 (750 uM, Stem Cell Technologies). The day after (day 3), cells are injected (0.5-1*10⁶ cells per mouse) i.v. in 9 to 10-week-old partially myeloablated immunodeficient NSG (NOD SCID GAMMA; NOD.Cg-Prkdc^{scid}Il2rg^{tm1Wjl}/SzJ) mice. After 16 weeks, mice are euthanized and bone marrow, thymus and spleen are analyzed for engraftment of human cells by flow cytometry using anti-human CD45 vs. anti-murine CD45 antibodies. The percentage of engrafted human cells is defined as follows: % huCD45+/(% huCD45++% muCD45+). Analysis of the different hematopoietic cell types present was performed by cell-specific staining for human CD34, human CD45, human CD19, human CD33, human CD71, human CD36 and human CD235a. Transduction efficiency and genome editing efficiency is determined in the purified HSPC and lymphoid and myeloid progeny, as described above in example 7.

Human CD34+ HSPC is isolated from bone marrow of engrafted mice using immunomagnetic separation (CD34 MicroBeads kit human; Miltenyi Biotech). The hCD34-positive fraction is cultured in 3-phase liquid erythroid culture system (Giarratana et al., Blood, 2011, 118(19):5071-9) or plated in a semi-solid medium containing cytokines supporting the growth of erythroid and myeloid hematopoietic progenitors (Clonal progenitor assay; medium GFH4435, Stem Cell Technologies). Given the low number of erythroid cells obtained in vivo in NSG mice, the expression of the anti-sickling transgene, the down-regulation of sickle beta-globin expression and the functional correction of the SCD phenotype are assessed ex vivo in the erythroid progeny of modified SCID-Repopulating cells, as describe above (example 7).

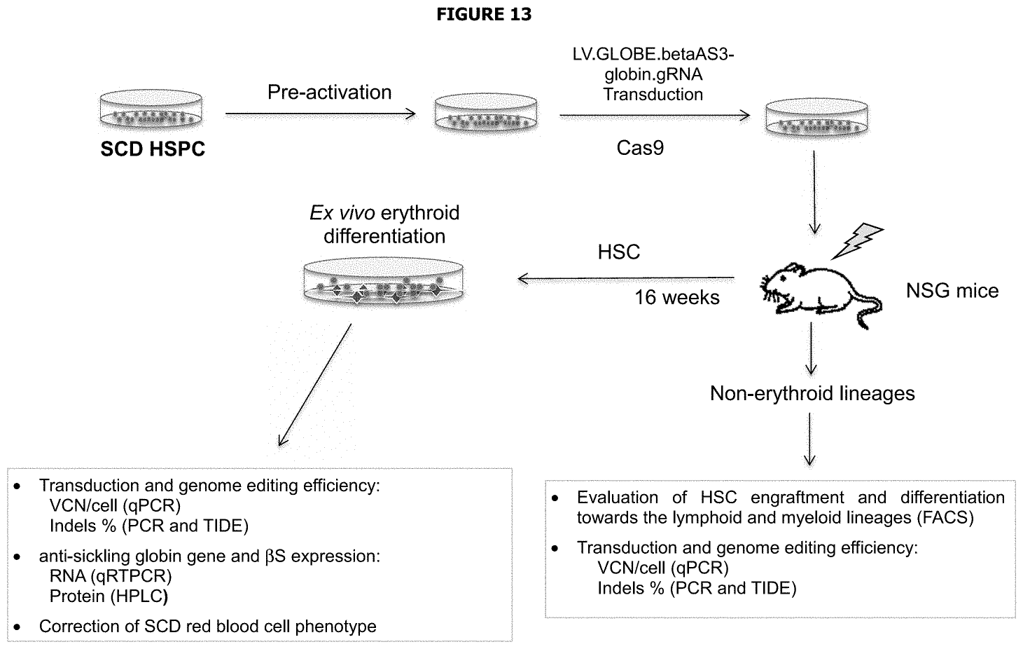

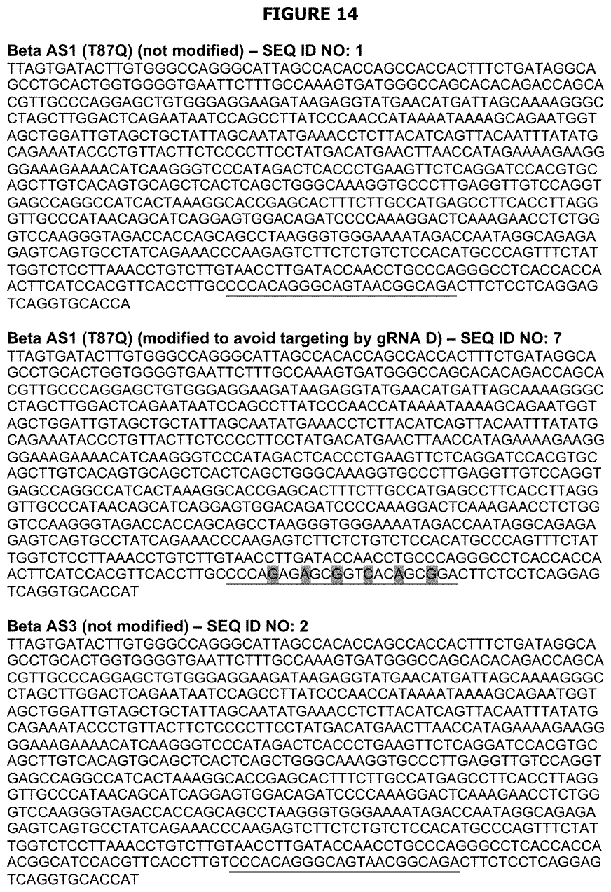

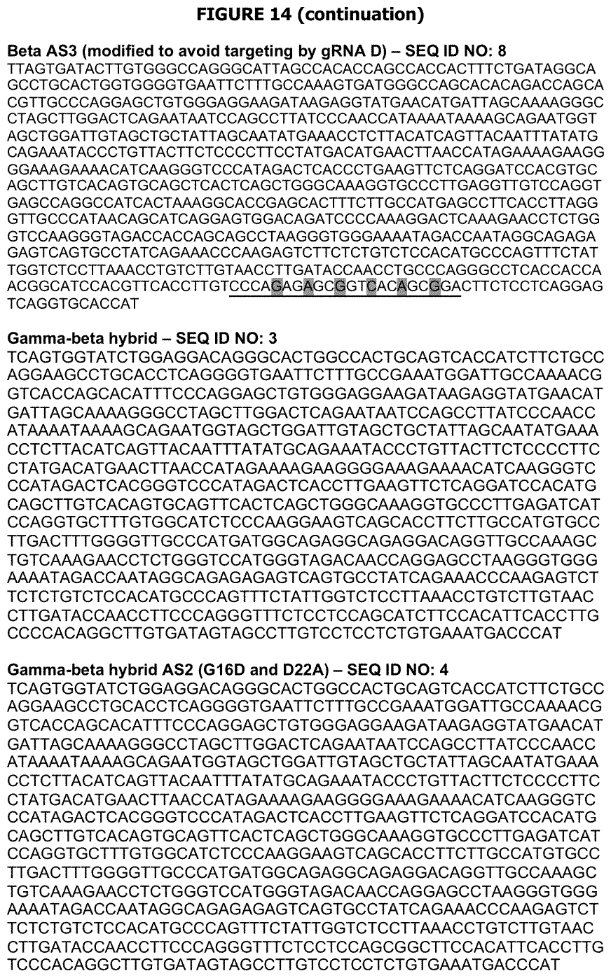

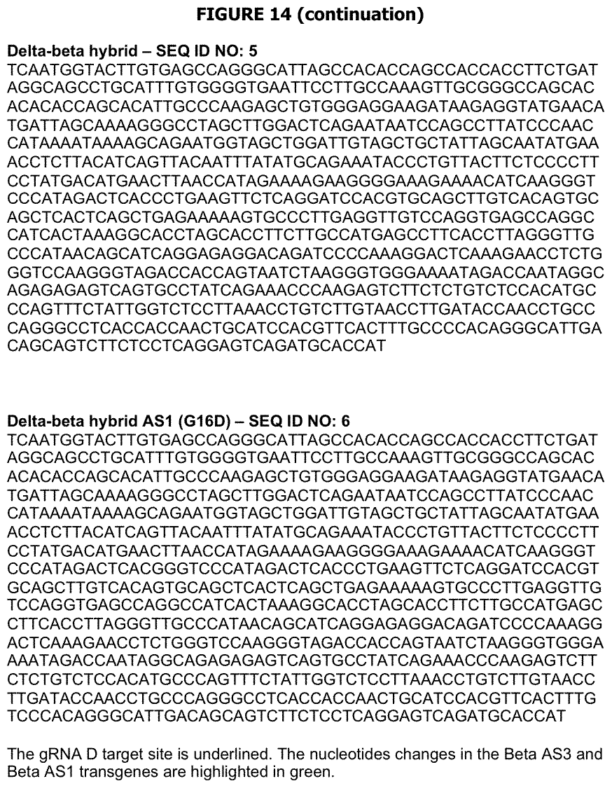

This process is illustrated in FIG. 13.

### Example 9: Evaluation of Transgene Expression, Genome Editing Efficiency and (i) Beta-Globin Down-Regulation (gRNA D) or (ii) Gamma-Globin Re-Activation (gRNA-13 bp-del and gRNA-BCL11Aenhancer)

### Protocols

### Lentiviral Vectors Used

LV.GLOBE-AS3modified (LV.GLOBE.betaAS3-globin plasmid (SEQ ID NO: 45): lentiviral vector harboring only a Beta-AS3 transgene modified by inserting silent mutations in the sequence of exon 1 targeted by gRNA-D (AS3modified transgene), does not express gRNAD

LV.GLOBE-AS3modified.gRNAD (LV.GLOBE-AS3modified.gRNAD, SEQ ID NO: 94): lentiviral vector expressing AS3modified transgene and optimized gRNA D.

LV.GLOBE-AS3modified.gRNA-luciferase (SEQ ID NO: 93): lentiviral vector expressing AS3modified transgene and optimized gRNA targeting the luciferase gene, which is not present in the human genome.

LV.GLOBE-AS3modified.gRNA-BCL11Aenhancer (SEQ ID NO: 75): lentiviral vector expressing AS3modified transgene and optimized BCL11A gRNA (5′-CACAGGCTCCAGGAAGGGTT-3′—SEQ ID NO: 74) targeting the intronic erythroid-specific enhancer of BCL11A gene. To evaluate the editing efficiency of BCL11A gRNA by TIDE the following primers were used:

BCL11A-TIDE FORWARD:
(SEQ ID NO: 77)
5′-TGGACAGCCCGACAGATGAA-3′
BCL11A-TIDE REVERSE:
(SEQ ID NO: 78)
5′-AAAAGCGATACAGGGCTGGC-3′

LV.GLOBE-AS3modified.gRNA-13 bp-del (SEQ ID NO: 76): lentiviral vector expressing AS3modified transgene and optimized 13 bp-del gRNA (SEQ ID NO: 71) designed to reproduce the 13 bp small HPFH deletion within the promoters of HBG1 and HBG2 genes. To evaluate the editing efficiency of 13 bp-del gRNA by TIDE the following primers were used:

13 bp-del-TIDE FORWARD:
(SEQ ID NO: 79)
5′-AAAAACGGCTGACAAAAGAAGTCCTGGTAT-3′
13 bp-del-TIDE REVERSE:
(SEQ ID NO: 80)
5′-ATAACCTCAGACGTTCCAGAAGCGAGTGTG-3′

### Transduction of HUDEP-2 Cells

HUDEP-2 WT cells were transduced at MOI 50 with LVs LV.GLOBE-AS3modified.gRNAD (D, SEQ ID NO: 94), LV.GLOBE-AS3modified.gRNA-BCL11Aenhancer (BCL11A, SEQ ID NO: 75) and LV.GLOBE-AS3modified.gRNA-13 bp-del (13bpdel, SEQ ID NO: 76).

Untransduced (UT) samples or cells transduced with LV.GLOBE-AS3modified (AS3, SEQ ID NO: 45) and LV.GLOBE-AS3modified.gRNA-luciferase (Luc) LVs were used as controls.

10 days after transduction, transduced cells were transfected using 4 μg GFP-Cas9 plasmid (pMJ920, Addgene plasmid #42234). After 18 hours plasmid-transfected Cas9-GFP+ cells (29%-45%, not shown) were sorted by FACS.

In parallel, an LVs LV.GLOBE-AS3modified.gRNAD-transduced sample was electroporated using 10 μg (60 pmol) of Cas9-GFP protein by using Nucleofector 4D (CA-137 program), achieving ≈90% of GFP+Cas9-expressing cells (not shown).

Sorted plasmid-transfected and unsorted Cas9-protein-transfected D samples, as well as non-transduced and non-transfected cells (UT) and transduced but non-transfected samples used as controls were then differentiated in mature erythroblasts.

mRNAs Quantification

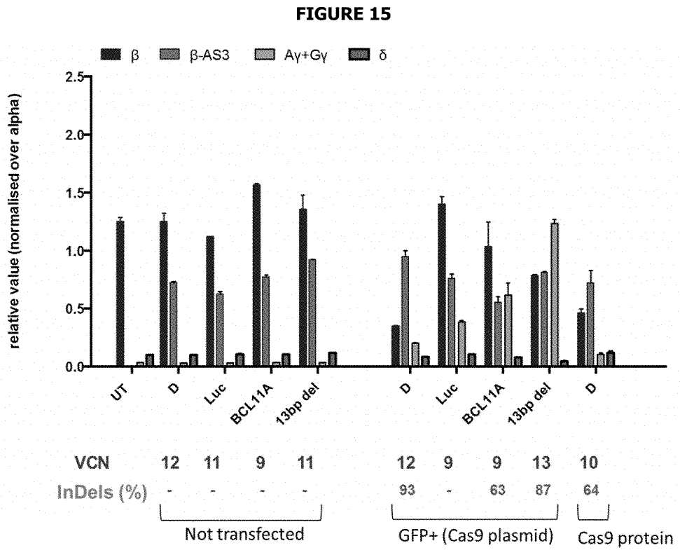

Globin mRNA expression in mature erythroblasts (day 9 of differentiation) is presented in FIG. 15.

Globin expression was evaluated by qRT-PCR in samples collected at day 9 of differentiation. Total RNA was extracted using RNeasy micro kit (QIAGEN) following manufacturer's instructions. Mature transcripts were reverse-transcribed using SuperScript First-Strand Synthesis System for RT-PCR (Invitrogen) with oligo(dT) primers. qRT-PCR was performed using SYBR green (Applied Biosystems).

Primers HBG1+HBG2 FORWARD: 5′-CCTGTCCTCTGCCTCTGCC-3′ (SEQ ID NO: 81) and HBG1+HBG2 REVERSE: 5′-GGATTGCCAAAACGGTCAC-3′ (SEQ ID NO: 82) were used to amplify the γ-globin transcripts. Primers HBB-AS3 FORWARD 5′-AAGGGCACCTTTGCCCAG-3′, (SEQ ID NO: 21) and HBB-AS3 REVERSE 5′—GCCACCACTTTCTGATAGGCAG-3′ (SEQ ID NO: 22) were used to amplify exclusively the beta AS3 globin transcripts. Primers HBB FORWARD: 5′-AAGGGCACCTTTGCCACA-3′, (SEQ ID NO: 81) and HBB REVERSE: 5′-gccaccactttctgataggcag-3′ (SEQ ID NO: 82) were used to amplify the endogenous β-globin transcripts. Primers HBD FORWARD: 5′-CAAGGGCACTTTTTCTCAG-3′ (SEQ ID NO: 85) and HBD REVERSE: 5′-AATTCCTTGCCAAAGTTGC-3′ (SEQ ID NO: 86) were used to amplify the δ-globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) were used to amplify the alpha-globin transcripts. Endogenous beta-globin, AS3 beta-globin, gamma-globin and delta-globin results were normalized to alpha-globin.

BCL11A mRNA expression in undifferentiated (day 0) HUDEP WT cells and in differentiated erythroblasts at different days of differentiation (day 5, day 7 and day 9) was evaluated by qRT-PCR (as described above) in samples transduced with LV.GLOBE-AS3modified.gRNA-BCL11Aenhancer with or without transfection with Cas9-GFP plasmids followed by flow cytometry-based selection of GFP+ cells. Time-course analysis of the total BCL11A mRNA isoforms and of the BCL11A isoform XL, mainly involved in the regulation of gamma-globin expression, was performed by using qRT-PCR with the following primers:

BCL11A FORWARD:
(SEQ ID NO: 87)
5′-AACCCCAGCACTTAAGCAAA-3′
BCL11A REVERSE:
(SEQ ID NO: 88)
5′-GGAGGTCATGATCCCCTTCT-3′
BC L11AXL FORWARD:
(SEQ ID NO: 89)
5′-ATGCGAGCTGTGCAACTATG-3′
BCL11AXL REVERSE:
(SEQ ID NO: 90)
5′-GTAAACGTCCTTCCCCACCT-3′
GAPDH FORWARD:
(SEQ ID NO: 91)
5′-CTTCATTGACCTCAACTACATGGTTT-3′
GAPDH REVERSE:
(SEQ ID NO: 92)
5′-TGGGATTTCCATTGATGACAAG-3′

### HPLC Analyses of Globin Chains and Hemoglobin Tetramers

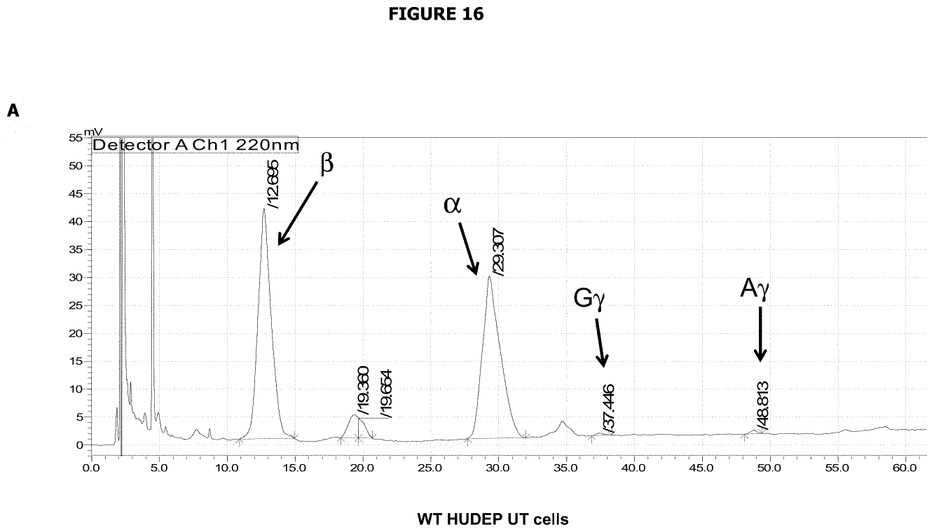

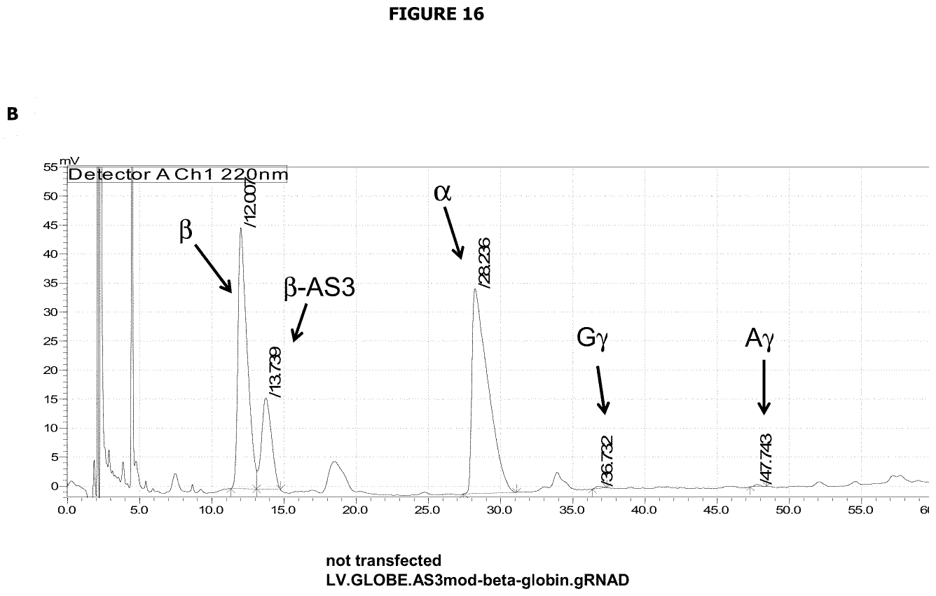

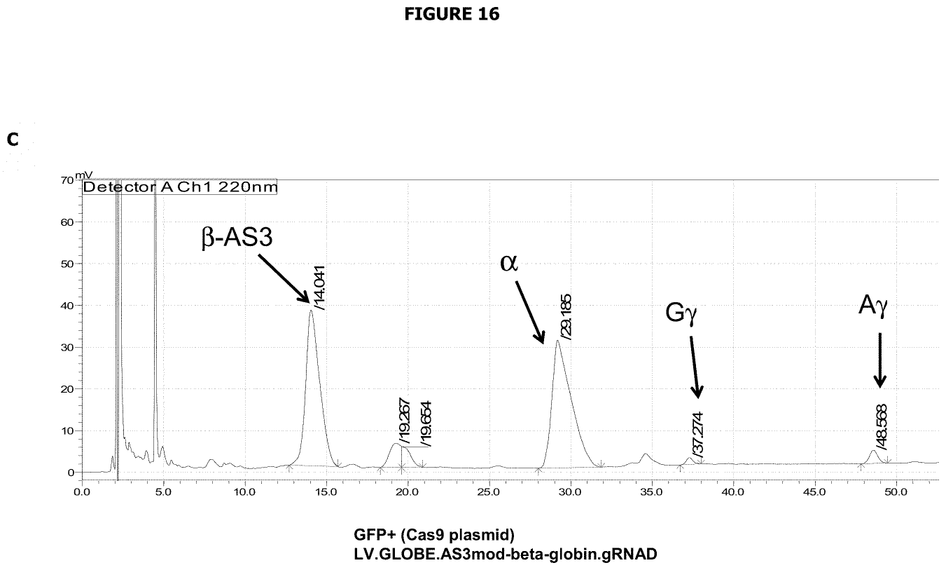

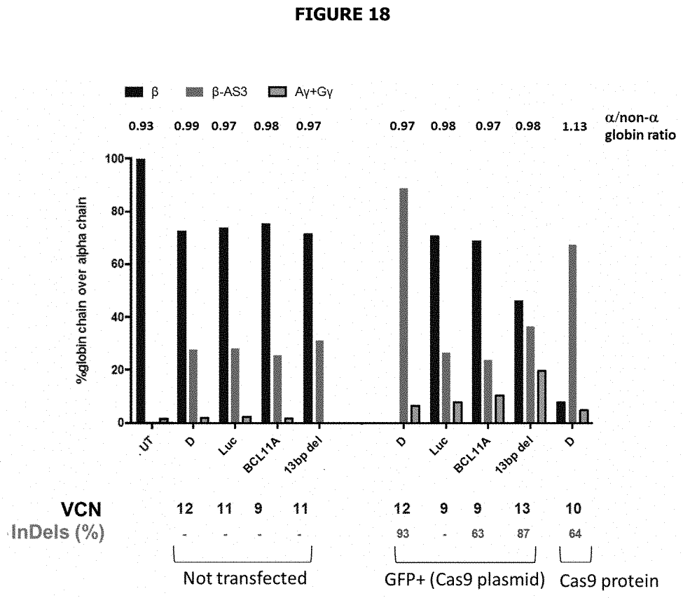

Globin chain profiles obtained using reverse phase HPLC in mature erythroblasts derived from control or genetically modified HUDEP cells (day 9 of differentiation) are presented in FIG. 16. Quantification of beta-like globin protein levels normalized to alpha-globin levels are shown in FIG. 18.

Briefly, reverse phase HPLC (RP-HPLC) analysis of globin chains was performed using a NexeraX2 SIL-30AC chromatograph (Shimadzu) and the LC Solution software. Globin chains from in vitro differentiated mature erythroblasts were separated by HPLC using a 250×4.6 mm, 3.6 μm Aeris Widepore column (Phenomenex). Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm.

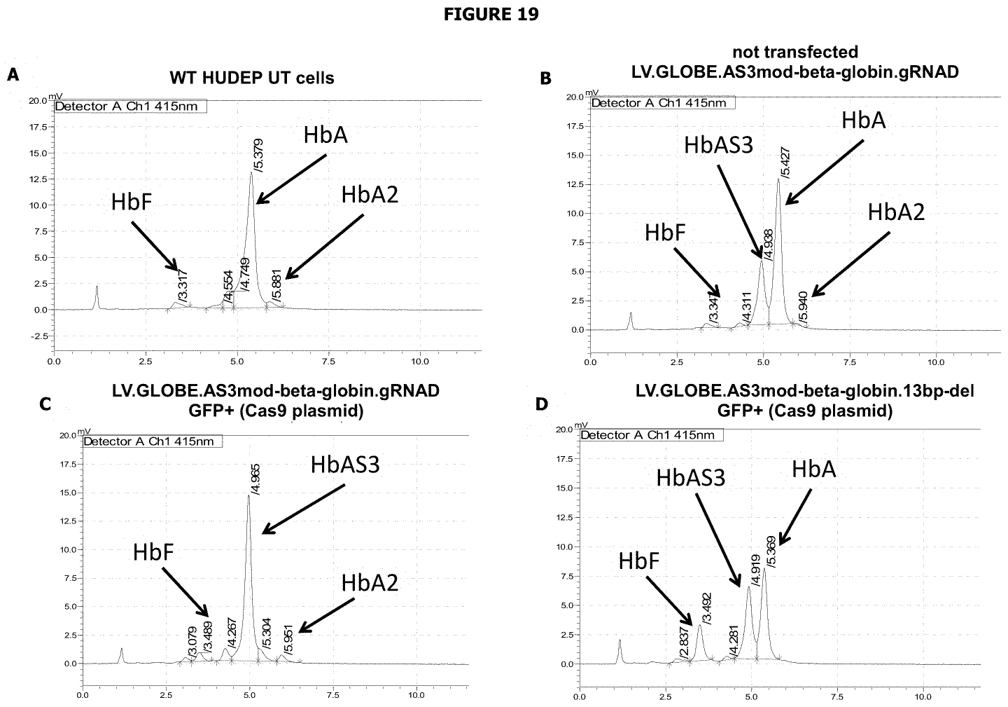

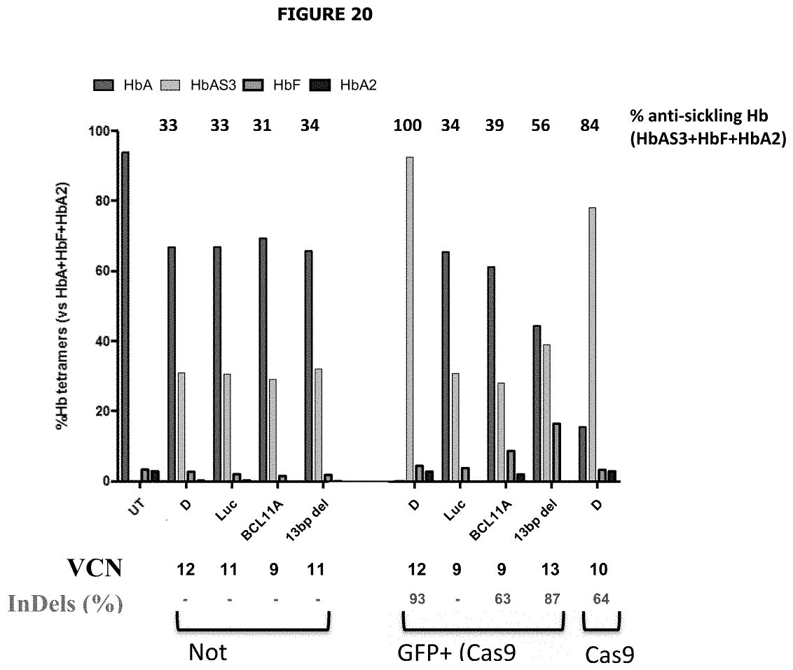

Hemoglobin profiles obtained using cation-exchange HPLC in mature erythroblasts derived from unmodified or genetically modified HUDEP cells (day 9 of differentiation) are presented in FIG. 19. Results of the quantification of each hemoglobin tetramer (HbA, HbAS3, HbF and HbA2) were reported as percentage over the total amount of hemoglobin tetramers and are shown in FIG. 20.

Analysis of hemoglobin tetramers was performed by cation-exchange HPLC using a NexeraX2 SIL-30AC chromatograph (Shimadzu) and the LC Solution software. Hemoglobin tetramers from mature erythroblasts were separated using a 2 cation-exchange column (PolyCAT A, PolyLC, Columbia, Md.). Samples were eluted with a gradient mixture of solution A (20 mM bis Tris, 2 mM KCN, pH=6.5) and solution B (20 mM bis Tris, 2 mM KCN, 250 mM NaCl, pH=6.8). The absorbance was measured at 415 nm.

### Results:

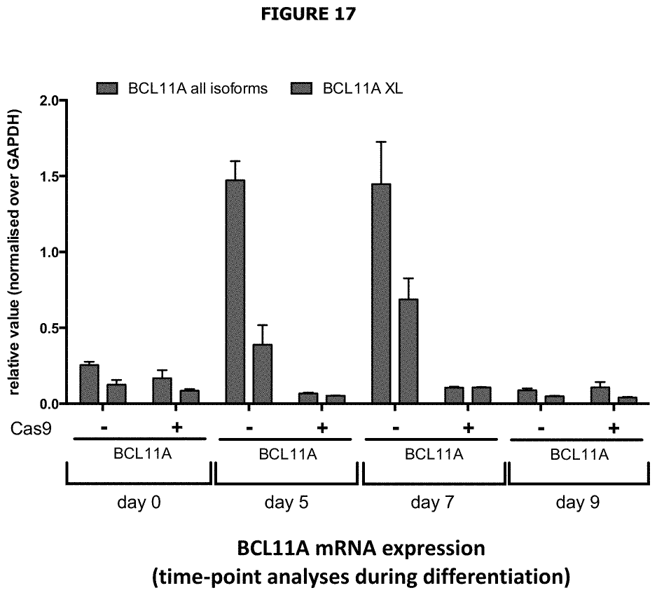

A) Globin (FIG. 15) and BCL11A mRNA Expression (FIG. 17)
1) Cells not transfected (Not transfected)

AS3mod (not shown in the figures): higher expression level of β-AS3 associated with the higher VCN compared to other samples.

“Luc” transduced cells: Similar expression level of endogenous HBB mRNA compared to controls (UT) and lower expression of AS3 beta-globin mRNA transgene compared to AS3mod due to lower VCN (FIG. 15).

“D” transduced cells: no inactivation of endogenous β-globin gene (i.e. HBB), due to the absence of Cas9 delivery. Similar expression level of endogenous HBB mRNA compared to controls (UT and “luc”). “D” also expresses AS3 beta-globin mRNA transgene at similar level compared to control (“luc”) with similar VCN (FIG. 15).

“BCL11A” and “13 bp del” transduced cells: no inactivation of endogenous β-globin gene (i.e. HBB), because of the expression of gRNAs that do not target HBB. Similar expression level of endogenous HBB mRNA in the BCL11A and 13 bp del samples compared to controls (UT and “luc”). Similar levels of expression of AS3 beta-globin mRNA transgene for both BCL11A and 13 bp del samples in comparison with control (“luc”) with similar VCN (FIG. 15).

Note that BCL11A/BCL11AXL mRNA expression levels are increased over-time with a peak at days 5 and 7 of differentiation in non-transfected BCL11A sample (used as control in FIG. 17).
2) Cells transfected with GFP-Cas9 plasmid (GFP+ (Cas9 plasmid)) or with Cas9-GFP protein (Cas9 protein)

AS3mod and Luc transduced cells: no genome editing in the exon 1 of endogenous HBB gene, as well as in the gamma-globin promoters or in the intronic enhancer of BCL11A gene, due to the absence of gRNAs in the LV vector (AS3mod) or the presence of a gRNA targeting the luciferase gene (Luc). Similar expression levels of endogenous beta-, AS3 beta-, gamma- and delta-globin chains compared to samples transduced with the same LV but «not transfected» with Cas9-GFP plasmid.

“D” transduced cells: down-regulation of endogenous β-globin gene expression in comparison with D «not transfected» sample and controls samples, due to the targeting of endogenous HBB gene by gRNA D and plasmid or protein delivery of Cas9. The expression of β-AS3 transgene and gamma-globin chains (Aγ+Gγ) tend to increase maybe as a consequence of HBB downregulation.

“BCL11A” and “13 bp del” transduced cells: an up-regulation of gamma-globin chains (Aγ+Gγ) expression is observed in comparison with “BCL11A” and “13 bp del” «not transfected» samples and controls, due to the disruption of the erythroid-specific BCL11A enhancer (BCL11A sample) or to the deletion of the 13-bp region in gamma-globin promoters (13 bp del sample) as a consequence of gRNA expression and plasmid delivery of Cas9. Indeed, the treatment with Cas9 strongly downregulated the expression of BCL11A, including XL isoform, in mature erythroblasts derived from Cas9-GFP+ BCL11A sample demonstrating that gRNA targeting the BCL11A enhancer is effective in decreasing BCL11A expression in erythroid cells and consequently implying a deregulation of γ-globin gene expression (see for example FIG. 15 or protein expression levels below). The 13 bp del sample showed reduced expression of the endogenous beta-globin gene. Similar expression levels of β-AS3- and delta-globin chains compared to samples transduced with the same LVs but «not transfected» with Cas9-GFP plasmid.

B) Protein Expression

HPLC analyses showed a dramatic down-regulation of endogenous beta-globin expression (“β”) and HbA tetramers (FIGS. 18 and 20) and increased amounts of exogenous β-AS3-globin and HbAS3 tetramers (FIGS. 18 and 20) in mature erythroblasts derived from HUDEP-2 cells transduced with LV.AS3-beta-globin.gRNAD and transfected with Cas9-GFP plasmid or Cas9 protein (FIG. 16 panel C and FIGS. 18 and 20), when compared LV.AS3-beta-globin.gRNAD transduced but non-transfected cells (FIG. 16 panel B and FIGS. 18 and 20).

In particular mature erythroblasts derived from HUDEP-2 cells transduced with LV.AS3-beta-globin.gRNA-D and transfected with Cas9-GFP plasmid or Cas9 protein showed almost a complete knock-down of endogenous beta-globin chain expression (“β”) and HbA tetramers compensated by the expression of exogenous β-AS3-globin expression and HbAS3 tetramers as demonstrated by the alpha/not-alpha ratio that is similar in control samples (FIGS. 18 and 20). Genome editing at HBB target site and, as a consequence, the reduction in endogenous beta-globin chain/HbA and the increase in beta-globin AS3/HbAS3, is VCN-dependent (not shown) but significant even at low VCN (VCN=3).

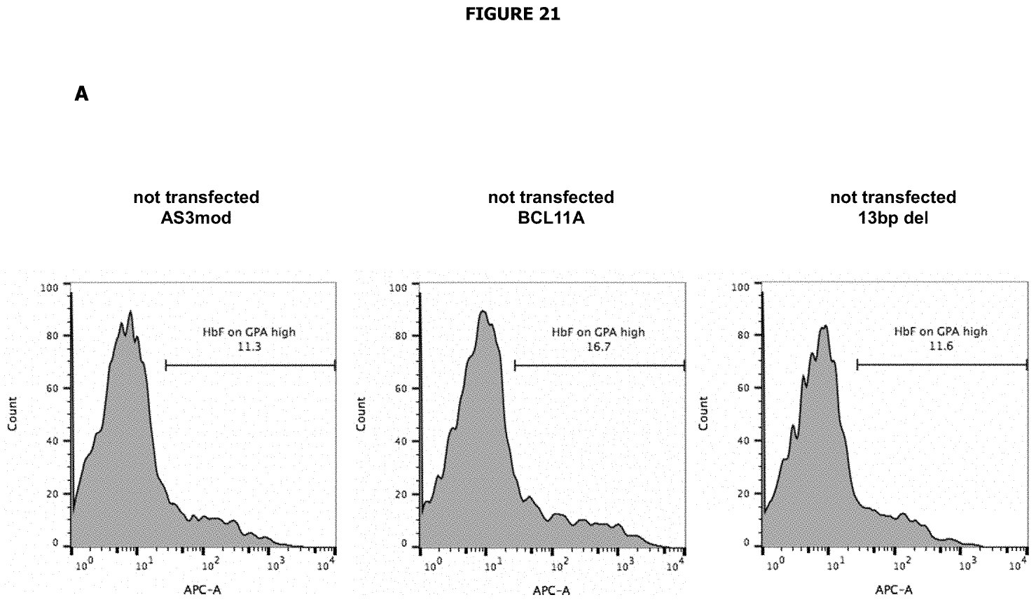

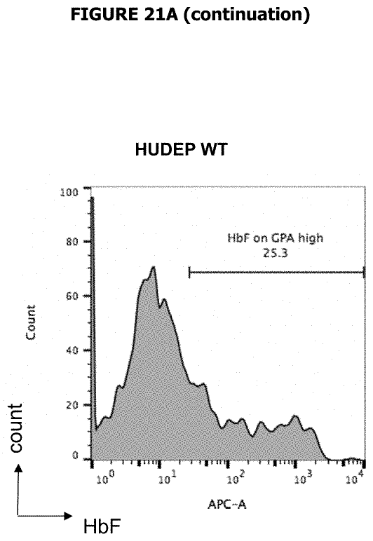

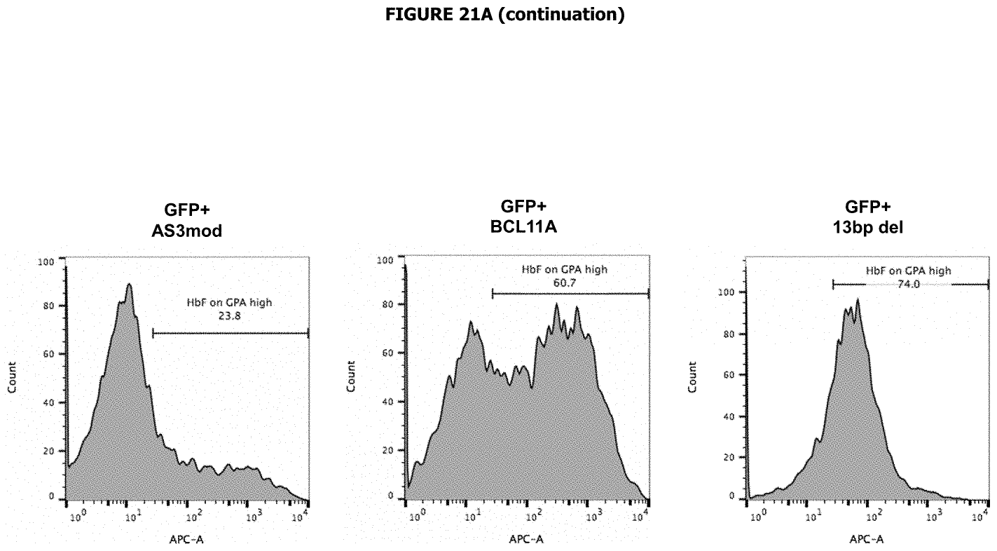

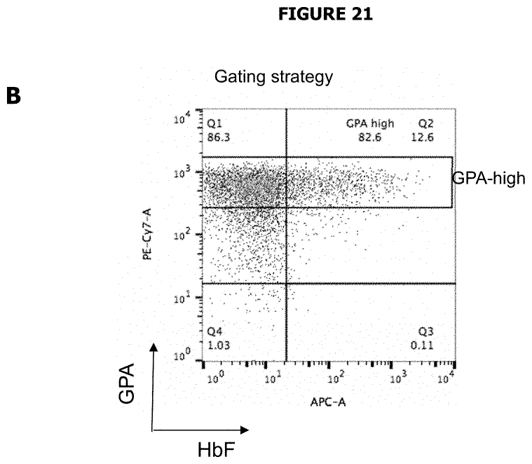

In mature erythroblasts derived from HUDEP-2 cells transduced with LV.AS3-beta-globin.gRNA-BCL11Aenhancer or LV.AS3-beta-globin.gRNA-13bpdel and transfected with Cas9-GFP plasmid, gamma-globin expression and HbF levels were significantly increased (FIGS. 18 and 20) compared to control samples and HbF expression pattern is close to be pan-cellular reaching 61% and 74% of F+ (HbF+) cells in mature erythroblasts derived from Cas9-expressing BCL11A and 13bpdel HUDEP-2, respectively (FIG. 21).

### Conclusions:

Transgene expression at mRNA and protein levels (FIGS. 18 and 20) are correlated and are not impaired by gRNA expression and Cas9 delivery. Transgene expression is correlated with VCN at both mRNA (FIG. 15) and protein levels (FIGS. 18 and 20).

In Cas9-GFP+D samples the knock-down of endogenous β-globin gene at mRNA level (FIG. 15) results in complete knock-out of endogenous β-globin protein expression (FIGS. 16 and 18) and absence of HbA tetramers (FIGS. 19 and 20). Hence a majority of anti-sickling tetramers (HbAS3) are observed in these cells.

The ratio between the expression of alpha-globin and non-alpha-globins (alpha/non-alpha ratio) is similar between all samples. The concomitant increase of anti-sickling globin expression (FIGS. 15-16), mainly AS3-β-globin (+60% in comparison with not-transfected D sample; FIGS. 16 and 19), compensates the observed robust endogenous β-globin downregulation. Hence, no modification in the balance between α- and other globin chain synthesis (FIGS. 18-19) is observed thereby avoiding generation of α-globin precipitates (FIGS. 19-20) which might be seen as a risk in the case of this therapeutic strategy.

Cas9 protein-mediated genome editing in “D” samples resulted in a clinically relevant switching between endogenous HbA tetramer (16%) and anti-sickling tetramers (HbAS3, HbF and HbA2; 84%) (FIGS. 19-20).

In mature erythroblasts derived from Cas9-GFP+13bpdel and BCL11A samples, a robust increase in γ-globin expression at both mRNA (FIG. 15) and protein (≈5 and ≈10 fold increase for 13bpdel and BCL11A, respectively; (FIG. 18)) levels in comparison with not-transfected 13bpdel and BCL11A samples was observed. Compared to matched non-transfected controls, in both 13bpdel and BCL11A samples an increased production of anti-sickling tetramers (+9% and +22% in 13bpdel and BCL11A, respectively; (FIGS. 19-20)) was observed, mainly associated with an enhanced generation of HbF tetramers. This finally resulted in ≈50% of HbA and ≈50% of HbAS3+HbF in 13bpdel sample, a condition resembling healthy heterozygous SCD carriers.

Relative amounts of HbA, HbA2, HbF and HbAS3 tetramers are shown in FIG. 20. Individuals with a level of anti-sickling Hb above 50% are considered healthy (i.e. HbAS3+HbF+HbA2), which is the case for erythroblasts derived from HUDEP-2 cells transduced with D or 13bpdel and transfected with Cas9.

All together these results showed the effectiveness of the integrative system as set up by the inventors in:
inactivating mutant beta-globin gene involved in SCD pathophysiology when gRNA D is used; and
expressing HbAS3 and, when gRNA BCL11A or gRNA 13bpdel are used instead of gRNAD, increasing expression of γ-globin chains, resulting in the production of an amount of antisickling hemoglobin tetramers sufficient to correct sickle cell disease and avoid alpha-globin precipitations.

1. A recombinant viral vector comprising in its genome:
(i) a nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder; and
(ii) a nucleotide sequence encoding a protein that has a therapeutic effect in said genetic disorder.

2. The recombinant viral vector according to claim 1, wherein the vector is a retroviral vector or an adeno-associated vector.

3. The recombinant viral vector according to claim 1, wherein the protein that has a therapeutic effect is an eukaryotic protein.

4. The recombinant viral vector according to claim 1, wherein the protein that has a therapeutic effect is selected from the group consisting of FGFR3, PBGD, SERPINA1, COL4A3, COL4A4, C9, f72, SOD1, TARDBP, FUS, ALS2, ANG, ATXN2, CHCHD10, CHMP2B, DCTN1, ERBB4, FIG4, HNRNPA1, MATR3, NEFH, OPTN, PFN1, PRPH, SETX, SIGMAR1, SMN1, SPG11, SQSTM1, TBK1, TRPM7, TUBA4A, UBQLN2, VAPB, VCP, CTLA4, NFKBIA, RHO, GNAT1, PDE6B, STAT3, PMP22, MPZ, LITAF, EGR2, NEFL, MFN2, KIF1B, RAB7A, LMNA, TRPV4, BSCL2, GARS, HSPB1, MPZ, GDAP1, HSPB8, DNM2, YARS, GJB1, PRPS1, STAT1, NFKB2, NFKB1, IKZF1, TNFRSF13B, ABCC8, KCNJ11, GLUD1, HADH, HNF1A, HNF4A, SLC16A1, UCP2, PTEN, SDHB, SDHD, KLLN, WT1, RHOA, TERC, THAP1, COL7A1, TOR1A, COL3A1, COL1A1, COL1A2, COL7A1, KRT5, KRT15, PLEC1, ITGB4, APC, BRCA1, RB1, FMR1, SLC40A1, ACVRL1, ENG, SMAD4, FH, BRCA1, BRCA2, HOXB13, REEP1, ATL1, SPAST, WASHC5, ANK1, EPB42, SLC4A1, SPTal, SPTB, HTT, STAT3, LDLR, APOB, PCSK9, SCN4A, CACNAlS, SCN4A, UNC119, PIK3CD, GATA2, IFNGR1, STAT1, STAT1, IRF8, PIK3R1, IFNAR2, BCL11B, TNFRSF13B, IKBKG, TWNK, p53, CHEK2, MLH1, MSH2, MSH6, PMS2, EPCAM, FBN1, HNF4A, GCK, HNF1A, PDX1, TCF2, NEURODI, KLF11, CEL, PAX4, INS, BLK, KCNJ11, APPL1, HIVEP2, MEN1, RET, CDKN1B, EXT1, EXT2, SGCE, DMPK, CNBP, NF1, NF2, ELANE, PTCH1, COL1A1, COL1A2, CRTAP, P3H1, STK11, PKD1, PKD2, ATP1A3, RHO, RP1, PRPH2RP9, IMPDH1, PRPF31, PRPF8, CA4, PRPF3, ABCA4, NRL, FSCN2, TOPORS, SNRNP200, SEMA4A, NR2E3, KLHL7, RGR, GUCA1B, BEST1, PRPF6, PRPF4, β-globin, γ-globi, δ-globin, β-globin harboring one Thr87Gln mutation, β-globin harboring three mutations Gly16Asp, Glu22Ala and Thr87Gln, γ-globin harboring two mutations Gly16Asp and Glu22Ala, δ-globin harboring one mutation Gly16Asp, VAPB, ATXN1, ATXN2, ATXN3, NOP56, CACNA1A, SC1, TSC2, VHL and VWF.

5. The recombinant viral vector according to claim 1, wherein the target gene is involved in the genetic disorder when said target gene is expressed in a patient.

6. The recombinant viral vector according to claim 1, wherein the target gene is selected from the group consisting of FGFR3, PBGD, SERPINA1, COL4A3, COL4A4, C9orf72, SOD1, TARDBP, FUS, ALS2, ANG, ATXN2, CHCHD10, CHMP2B, DCTN1, ERBB4, FIG4, HNRNPA1, MATR3, NEFH, OPTN, PFN1, PRPH, SETX, SIGMAR1, SMN1, SPG11, SQSTM1, TBK1, TRPM7, TUBA4A, UBQLN2, VAPB, VCP, CTLA4, NFKBIA, RHO, GNAT1, PDE6B, STAT3, PMP22, MPZ, LITAF, EGR2, NEFL, MFN2, KIF1B, RAB7A, LMNA, TRPV4, BSCL2, GARS, HSPB1, MPZ, GDAP1, HSPB8, DNM2, YARS, GJB1, PRPS1, STAT1, NFKB2, NFKB1, IKZF1, TNFRSF13B, ABCC8, KCNJ11, GLUD1, HADH, HNF1A, HNF4A, SLC16A1, UCP2, PTEN, SDHB, SDHD, KLLN, WT1, RHOA, TERC, THAP1, COL7A1, TOR1A, COL3A1, COL1A1, COL1A2, COL7A1, KRT5, KRT15, PLEC1, ITGB4, APC, BRCA1, RB1, FMR1, SLC40A1, ACVRL1, ENG, SMAD4, FH, BRCA1, BRCA2 or HOXB13, REEP1, ATL1, SPAST, WASHC5, ANK1, EPB42, SLC4A1, SPTal, SPTB, HTT, STAT3, LDLR, APOB, PCSK9, SCN4A, CACNAlS, SCN4A, UNC119, PIK3CD, GATA2, IFNGR1, STAT1, STAT1, IRF8, PIK3R1, IFNAR2, BCL11B, TNFRSF13B, IKBKG, TWNK, TP53, CHEK2, MLH1, MSH2, MSH6, PMS2, EPCAM, FBN1, HNF4A, GCK, HNF1A, PDX1, TCF2, NEURODI, KLF11, CEL, PAX4, INS, BLK, KCNJ11, APPL1, HIVEP2, MEN1, RET, CDKN1B, EXT1, EXT2, SGCE, DMPK, CNBP, NF1, NF2, ELANE, PTCH1, COL1A1, COL1A2, CRTAP, P3H1, STK11, PKD1, PKD2, ATP1A3, RHO, RP1, PRPH2RP9, IMPDH1, PRPF31, PRPF8, CA4, PRPF3, ABCA4, NRL, FSCN2, TOPORS, SNRNP200, SEMA4A, NR2E3, KLHL7, RGR, GUCA1B, BEST1, PRPF6, PRPF4, β-globin, VAPB, ATXN1, ATXN2, ATXN3, NOP56, CACNA1A, SC1, TSC2, VHL, BCL11A and VWF.

7. The recombinant viral vector according to claim 1, wherein the genetic disorder is selected from the group consisting of:
Achondroplasia
acute intermittent porphyria
Alpha-1 antitrypsin deficiency
Alport syndrome
Amyotrophic lateral sclerosis
autoimmune lymphoproliferative syndrome type V
autosomal dominant anhidrotic ectodermal dysplasia with T-cell
immunodeficiency
Autosomal dominant congenital stationary night blindness
Autosomal dominant hyper-IgE syndrome
Charcot-Marie-Tooth
Chronic Mucocutaneous Candidiasis
Common variable immune deficiency 10
Common variable immune deficiency 12
Common variable immune deficiency 13
Common variable immune deficiency 2
Congenital hyperinsulinism
Cowden syndrome
Denys-Drash syndrome
Diffuse-type gastric carcinoma
dyskeratosis congenita-1
Dystonia 6
dystrophic epidermolysis bullosa pruriginosa
Early-onset primary dystonia
Ehlers-Danlos syndrome type IV
Ehlers-Danlos syndrome type VII
epidermolysis bullosa dystrophica
epidermolysis bullosa simplex
Familial adenomatous polyposis
familial breast-ovarian cancer-1
familial retinoblastoma
Fragile X syndrome
Hereditary hemochromatosis type 4
Hereditary hemorrhagic telangiectasia
Hereditary leiomyomatosis and renal cell cancer
Hereditary prostate cancer
hereditary spastic paraplegia type 31
hereditary spastic paraplegia type 3A
hereditary spastic paraplegia type 4
hereditary spastic paraplegia type 8
Hereditary spherocytosis
Huntington disease
hyper-IgE recurrent infection syndrome
Hypercholesterolemia
Hyperkalemic periodic paralysis
Hypokalemic periodic paralysis
immunodeficiency-13
immunodeficiency-14
immunodeficiency-21
immunodeficiency-27B
immunodeficiency-31A
immunodeficiency-31C
immunodeficiency-32A
immunodeficiency-36
immunodeficiency-45
immunodeficiency-49
Immunoglobulin A (IgA) deficiency-2
Incontinentia pigmenti
Infantile-onset spinocerebellar ataxia
Li-Fraumeni syndrome
Lynch syndrome
Marfan syndrome
maturity-onset diabetes of the young
mental retardation-43
Multiple endocrine neoplasia
Multiple exostoses type I
Multiple exostoses type II
Myoclonus-dystonia
Myotonic dystrophy
Neurofibromatosis type 1
Neurofibromatosis type 2
neutropenia-1
nevoid basal cell carcinoma syndrome
Osteogenesis imperfecta
Peutz-Jeghers syndrome
Polycystic kidney disease
Rapid-onset dystonia parkinsonism
Retinitis pigmentosa
sickle cell disorder
Spinal muscular atrophy, lower extremity, dominant (SMA-LED) and
adult-onset form of spinal muscular atrophy
Spinocerebellar ataxia type 1
Spinocerebellar ataxia type 2
Spinocerebellar ataxia type 3
Spinocerebellar ataxia type 36
Spinocerebellar ataxia type 6
Tuberous sclerosis complex
Von Hippel-Lindau syndrome
Von Willebrand disease type I and II

8. A composition comprising a recombinant viral vector according to claim 1 or a plurality of said recombinant viral vectors.

9. A kit comprising:
a recombinant viral vector according to claim 1; and
a catalytically active Cas9 or Cpf1 protein or a nucleotide sequence encoding a catalytically active Cas9 or Cpf1 protein.

10. The recombinant viral vector according to claim 1 for introducing into a cell (i) nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder and (ii) a nucleotide sequence encoding a protein that has a therapeutic effect in said genetic disorder.

11. A method for modifying the genome of a cell in vitro or ex vivo, comprising the steps of:
a) contacting a cell with a recombinant viral vector of claim 1 to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

12. A method for preparing a genetically modified cell in vitro or ex vivo, comprising the steps of:
a) contacting a cell with a recombinant viral vector of claim 1 to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

13. The method according to claim 11, wherein the cell is an eukaryotic cell.

14. The method according to claim 11, wherein the cell is a stem cell, a progenitor cell or a differentiated cell.

15. A genetically modified cell obtainable by the method according to claim 11.

16. A medicament comprising a genetically modified cell obtainable by the method according to claim 11.

17. A method for treating a genetic disorder selected from the group consisting of:
Achondroplasia
acute intermittent porphyria
Alpha-1 antitrypsin deficiency
Alport syndrome
Amyotrophic lateral sclerosis
autoimmune lymphoproliferative syndrome type V
autosomal dominant anhidrotic ectodermal dysplasia with T-cell
immunodeficiency
Autosomal dominant congenital stationary night blindness
Autosomal dominant hyper-IgE syndrome
Charcot-Marie-Tooth
Chronic Mucocutaneous Candidiasis
Common variable immune deficiency 10
Common variable immune deficiency 12
Common variable immune deficiency 13
Common variable immune deficiency 2
Congenital hyperinsulinism
Cowden syndrome
Denys-Drash syndrome
Diffuse-type gastric carcinoma
dyskeratosis congenita-1
Dystonia 6
dystrophic epidermolysis bullosa pruriginosa
Early-onset primary dystonia
Ehlers-Danlos syndrome type IV
Ehlers-Danlos syndrome type VII
epidermolysis bullosa dystrophica
epidermolysis bullosa simplex
Familial adenomatous polyposis
familial breast-ovarian cancer-1
familial retinoblastoma
Fragile X syndrome
Hereditary hemochromatosis type 4
Hereditary hemorrhagic telangiectasia
Hereditary leiomyomatosis and renal cell cancer
Hereditary prostate cancer
hereditary spastic paraplegia type 31
hereditary spastic paraplegia type 3A
hereditary spastic paraplegia type 4
hereditary spastic paraplegia type 8
Hereditary spherocytosis
Huntington disease
hyper-IgE recurrent infection syndrome
Hypercholesterolemia
Hyperkalemic periodic paralysis
Hypokalemic periodic paralysis
immunodeficiency-13
immunodeficiency-14
immunodeficiency-21
immunodeficiency-27B
immunodeficiency-31A
immunodeficiency-31C
immunodeficiency-32A
immunodeficiency-36
immunodeficiency-45
immunodeficiency-49
Immunoglobulin A (IgA) deficiency-2
Incontinentia pigmenti
Infantile-onset spinocerebellar ataxia
Li-Fraumeni syndrome
Lynch syndrome
Marfan syndrome
maturity-onset diabetes of the young
mental retardation-43
Multiple endocrine neoplasia
Multiple exostoses type I
Multiple exostoses type II
Myoclonus-dystonia
Myotonic dystrophy
Neurofibromatosis type 1
Neurofibromatosis type 2
neutropenia-1
nevoid basal cell carcinoma syndrome
Osteogenesis imperfecta
Peutz-Jeghers syndrome
Polycystic kidney disease
Rapid-onset dystonia parkinsonism
Retinitis pigmentosa
sickle cell disorder
Spinal muscular atrophy, lower extremity, dominant (SMA-LED)
and adult-onset form of spinal muscular atrophy
Spinocerebellar ataxia type 1
Spinocerebellar ataxia type 2
Spinocerebellar ataxia type 3
Spinocerebellar ataxia type 36
Spinocerebellar ataxia type 6
Tuberous sclerosis complex
Von Hippel-Lindau syndrome
Von Willebrand disease type I and II
comprising administering a genetically modified cell obtainable by the method according to claim 11.

18. A method for treating sickle cell disorder (SCD) comprising administering a genetically modified cell obtainable by the method according to claim 11.

19. A kit comprising:
a composition according to claim 8; and
a catalytically active Cas9 or Cpf1 protein or a nucleotide sequence encoding a catalytically active Cas9 or Cpf1 protein.

20. The composition according to claim 8 for introducing into a cell (i) nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder and (ii) a nucleotide sequence encoding a protein that has a therapeutic effect in said genetic disorder.

21. The kit according to claim 9 for use in introducing into a cell (i) nucleotide sequence encoding a guide RNA (gRNA) that comprises a spacer adapted to bind to a target nucleotide sequence, said target nucleotide sequence is within the coding sequence of a target gene, within a transcribed non-coding sequence of a target gene or within a non-transcribed sequence, either upstream or downstream, of a target gene, said target gene is involved in a genetic disorder and (ii) a nucleotide sequence encoding a protein that has a therapeutic effect in said genetic disorder.

22. A method for modifying the genome of a cell in vitro or ex vivo, comprising the steps of:
a) contacting a cell with a composition of claim 8 to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

23. A method for preparing a genetically modified cell in vitro or ex vivo, comprising the steps of:
a) contacting a cell with a composition of claim 8 to obtain a transduced cell; and
b) introducing into the transduced cell a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced cell.

24. The method according to claim 12, wherein the cell is an eukaryotic cell.

25. The method according to claim 22, wherein the cell is an eukaryotic cell.

26. The method according to claim 23, wherein the cell is an eukaryotic cell.

27. The method according to claim 12, wherein the cell is a stem cell, a progenitor cell or a differentiated cell.

28. The method according to claim 22, wherein the cell is a stem cell, a progenitor cell or a differentiated cell.

29. The method according to claim 23, wherein the cell is a stem cell, a progenitor cell or a differentiated cell.

30. A genetically modified cell obtainable by the method according to claim 12.

31. A genetically modified cell obtainable by the method according to claim 22.

32. A genetically modified cell obtainable by the method according to claim 23.

33. A medicament comprising a genetically modified cell obtainable by the method according to claim 12.

34. A medicament comprising a genetically modified cell obtainable by the method according to claim 22.

35. A medicament comprising a genetically modified cell obtainable by the method according to claim 23.

36. New A method for treating a genetic disorder selected from the group consisting of:
Achondroplasia
acute intermittent porphyria
Alpha-1 antitrypsin deficiency
Alport syndrome
Amyotrophic lateral sclerosis
autoimmune lymphoproliferative syndrome type V
autosomal dominant anhidrotic ectodermal dysplasia with T-cell
immunodeficiency
Autosomal dominant congenital stationary night blindness
Autosomal dominant hyper-IgE syndrome
Charcot-Marie-Tooth
Chronic Mucocutaneous Candidiasis
Common variable immune deficiency 10
Common variable immune deficiency 12
Common variable immune deficiency 13
Common variable immune deficiency 2
Congenital hyperinsulinism
Cowden syndrome
Denys-Drash syndrome
Diffuse-type gastric carcinoma
dyskeratosis congenita-1
Dystonia 6
dystrophic epidermolysis bullosa pruriginosa
Early-onset primary dystonia
Ehlers-Danlos syndrome type IV
Ehlers-Danlos syndrome type VII
epidermolysis bullosa dystrophica
epidermolysis bullosa simplex
Familial adenomatous polyposis
familial breast-ovarian cancer-1
familial retinoblastoma
Fragile X syndrome
Hereditary hemochromatosis type 4
Hereditary hemorrhagic telangiectasia
Hereditary leiomyomatosis and renal cell cancer
Hereditary prostate cancer
hereditary spastic paraplegia type 31
hereditary spastic paraplegia type 3A
hereditary spastic paraplegia type 4
hereditary spastic paraplegia type 8
Hereditary spherocytosis
Huntington disease
hyper-IgE recurrent infection syndrome
Hypercholesterolemia
Hyperkalemic periodic paralysis
Hypokalemic periodic paralysis
immunodeficiency-13
immunodeficiency-14
immunodeficiency-21
immunodeficiency-27B
immunodeficiency-31A
immunodeficiency-31C
immunodeficiency-32A
immunodeficiency-36
immunodeficiency-45
immunodeficiency-49
Immunoglobulin A (IgA) deficiency-2
Incontinentia pigmenti
Infantile-onset spinocerebellar ataxia
Li-Fraumeni syndrome
Lynch syndrome
Marfan syndrome
maturity-onset diabetes of the young
mental retardation-43
Multiple endocrine neoplasia
Multiple exostoses type I
Multiple exostoses type II
Myoclonus-dystonia
Myotonic dystrophy
Neurofibromatosis type 1
Neurofibromatosis type 2
neutropenia-1
nevoid basal cell carcinoma syndrome
Osteogenesis imperfecta
Peutz-Jeghers syndrome
Polycystic kidney disease
Rapid-onset dystonia parkinsonism
Retinitis pigmentosa
sickle cell disorder
Spinal muscular atrophy, lower extremity, dominant (SMA-LED)
and adult-onset form of spinal muscular atrophy
Spinocerebellar ataxia type 1
Spinocerebellar ataxia type 2
Spinocerebellar ataxia type 3
Spinocerebellar ataxia type 36
Spinocerebellar ataxia type 6
Tuberous sclerosis complex
Von Hippel-Lindau syndrome
Von Willebrand disease type I and II
comprising administering a genetically modified cell obtainable by the method according to claim 12.

37. A method for treating a genetic disorder selected from the group consisting of:
Achondroplasia
acute intermittent porphyria
Alpha-1 antitrypsin deficiency
Alport syndrome
Amyotrophic lateral sclerosis
autoimmune lymphoproliferative syndrome type V
autosomal dominant anhidrotic ectodermal dysplasia with T-cell
immunodeficiency
Autosomal dominant congenital stationary night blindness
Autosomal dominant hyper-IgE syndrome
Charcot-Marie-Tooth
Chronic Mucocutaneous Candidiasis
Common variable immune deficiency 10
Common variable immune deficiency 12
Common variable immune deficiency 13
Common variable immune deficiency 2
Congenital hyperinsulinism
Cowden syndrome
Denys-Drash syndrome
Diffuse-type gastric carcinoma
dyskeratosis congenita-1
Dystonia 6
dystrophic epidermolysis bullosa pruriginosa
Early-onset primary dystonia
Ehlers-Danlos syndrome type IV
Ehlers-Danlos syndrome type VII
epidermolysis bullosa dystrophica
epidermolysis bullosa simplex
Familial adenomatous polyposis
familial breast-ovarian cancer-1
familial retinoblastoma
Fragile X syndrome
Hereditary hemochromatosis type 4
Hereditary hemorrhagic telangiectasia
Hereditary leiomyomatosis and renal cell cancer
Hereditary prostate cancer
hereditary spastic paraplegia type 31
hereditary spastic paraplegia type 3A
hereditary spastic paraplegia type 4
hereditary spastic paraplegia type 8
Hereditary spherocytosis
Huntington disease
hyper-IgE recurrent infection syndrome
Hypercholesterolemia
Hyperkalemic periodic paralysis
Hypokalemic periodic paralysis
immunodeficiency-13
immunodeficiency-14
immunodeficiency-21
immunodeficiency-27B
immunodeficiency-31A
immunodeficiency-31C
immunodeficiency-32A
immunodeficiency-36
immunodeficiency-45
immunodeficiency-49
Immunoglobulin A (IgA) deficiency-2
Incontinentia pigmenti
Infantile-onset spinocerebellar ataxia
Li-Fraumeni syndrome
Lynch syndrome
Marfan syndrome
maturity-onset diabetes of the young
mental retardation-43
Multiple endocrine neoplasia
Multiple exostoses type I
Multiple exostoses type II
Myoclonus-dystonia
Myotonic dystrophy
Neurofibromatosis type 1
Neurofibromatosis type 2
neutropenia-1
nevoid basal cell carcinoma syndrome
Osteogenesis imperfecta
Peutz-Jeghers syndrome
Polycystic kidney disease
Rapid-onset dystonia parkinsonism
Retinitis pigmentosa
sickle cell disorder
Spinal muscular atrophy, lower extremity, dominant (SMA-LED)
and adult-onset form of spinal muscular atrophy
Spinocerebellar ataxia type 1
Spinocerebellar ataxia type 2
Spinocerebellar ataxia type 3
Spinocerebellar ataxia type 36
Spinocerebellar ataxia type 6
Tuberous sclerosis complex
Von Hippel-Lindau syndrome
Von Willebrand disease type I and II
comprising administering a genetically modified cell obtainable by the method according to claim 22.

38. A method for treating a genetic disorder selected from the group consisting of:
Achondroplasia
acute intermittent porphyria
Alpha-1 antitrypsin deficiency
Alport syndrome
Amyotrophic lateral sclerosis
autoimmune lymphoproliferative syndrome type V
autosomal dominant anhidrotic ectodermal dysplasia with T-cell
immunodeficiency
Autosomal dominant congenital stationary night blindness
Autosomal dominant hyper-IgE syndrome
Charcot-Marie-Tooth
Chronic Mucocutaneous Candidiasis
Common variable immune deficiency 10
Common variable immune deficiency 12
Common variable immune deficiency 13
Common variable immune deficiency 2
Congenital hyperinsulinism
Cowden syndrome
Denys-Drash syndrome
Diffuse-type gastric carcinoma
dyskeratosis congenita-1
Dystonia 6
dystrophic epidermolysis bullosa pruriginosa
Early-onset primary dystonia
Ehlers-Danlos syndrome type IV
Ehlers-Danlos syndrome type VII
epidermolysis bullosa dystrophica
epidermolysis bullosa simplex
Familial adenomatous polyposis
familial breast-ovarian cancer-1
familial retinoblastoma
Fragile X syndrome
Hereditary hemochromatosis type 4
Hereditary hemorrhagic telangiectasia
Hereditary leiomyomatosis and renal cell cancer
Hereditary prostate cancer
hereditary spastic paraplegia type 31
hereditary spastic paraplegia type 3A
hereditary spastic paraplegia type 4
hereditary spastic paraplegia type 8
Hereditary spherocytosis
Huntington disease
hyper-IgE recurrent infection syndrome
Hypercholesterolemia
Hyperkalemic periodic paralysis
Hypokalemic periodic paralysis
immunodeficiency-13
immunodeficiency-14
immunodeficiency-21
immunodeficiency-27B
immunodeficiency-31A
immunodeficiency-31C
immunodeficiency-32A
immunodeficiency-36
immunodeficiency-45
immunodeficiency-49
Immunoglobulin A (IgA) deficiency-2
Incontinentia pigmenti
Infantile-onset spinocerebellar ataxia
Li-Fraumeni syndrome
Lynch syndrome
Marfan syndrome
maturity-onset diabetes of the young
mental retardation-43
Multiple endocrine neoplasia
Multiple exostoses type I
Multiple exostoses type II
Myoclonus-dystonia
Myotonic dystrophy
Neurofibromatosis type 1
Neurofibromatosis type 2
neutropenia-1
nevoid basal cell carcinoma syndrome
Osteogenesis imperfecta
Peutz-Jeghers syndrome
Polycystic kidney disease
Rapid-onset dystonia parkinsonism
Retinitis pigmentosa
sickle cell disorder
Spinal muscular atrophy, lower extremity, dominant (SMA-LED)
and adult-onset form of spinal muscular atrophy
Spinocerebellar ataxia type 1
Spinocerebellar ataxia type 2
Spinocerebellar ataxia type 3
Spinocerebellar ataxia type 36
Spinocerebellar ataxia type 6
Tuberous sclerosis complex
Von Hippel-Lindau syndrome
Von Willebrand disease type I and II
comprising administering a genetically modified cell obtainable by the method according to claim 23.

39. A method for treating sickle cell disorder (SCD) comprising administering a genetically modified cell obtainable by the method according to claim 12.

40. A method for treating sickle cell disorder (SCD) comprising administering a genetically modified cell obtainable by the method according to claim 22.

41. A method for treating sickle cell disorder (SCD) comprising administering a genetically modified cell obtainable by the method according to claim 23.


SEQUENCE LISTING (94 sequences)

SEQ ID NO: 1 - DNA, 830 bp, Artificial Sequence
  Beta AS1 (T87Q) (not modified)
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcacca

SEQ ID NO: 2 - DNA, 831 bp, Artificial Sequence
  Beta AS3 (not modified)
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccat

SEQ ID NO: 3 - DNA, 848 bp, Artificial Sequence
  Gamma-beta hybrid
tcagtggtatctggaggacagggcactggccactgcagtcaccatcttctgccaggaagcctgcacctcaggggtgaattctttgccgaaatggattgccaaaacggtcaccagcacatttcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcacgggtcccatagactcaccttgaagttctcaggatccacatgcagcttgtcacagtgcagttcactcagctgggcaaaggtgcccttgagatcatccaggtgctttgtggcatctcccaaggaagtcagcaccttcttgccatgtgccttgactttggggttgcccatgatggcagaggcagaggacaggttgccaaagctgtcaaagaacctctgggtccatgggtagacaaccaggagcctaagggtgggaaaatagaccaataggcagagagagtcagtgcctatcagaaacccaagagtcttctctgtctccacatgcccagtttctattggtctccttaaacctgtcttgtaaccttgataccaaccttcccagggtttctcctccagcatcttccacattcaccttgccccacaggcttgtgatagtagccttgtcctcctctgtgaaatgacccat

SEQ ID NO: 4 - DNA, 848 bp, Artificial Sequence
  Gamma-beta hybrid AS2 (G16D and D22A)
tcagtggtatctggaggacagggcactggccactgcagtcaccatcttctgccaggaagcctgcacctcaggggtgaattctttgccgaaatggattgccaaaacggtcaccagcacatttcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcacgggtcccatagactcaccttgaagttctcaggatccacatgcagcttgtcacagtgcagttcactcagctgggcaaaggtgcccttgagatcatccaggtgctttgtggcatctcccaaggaagtcagcaccttcttgccatgtgccttgactttggggttgcccatgatggcagaggcagaggacaggttgccaaagctgtcaaagaacctctgggtccatgggtagacaaccaggagcctaagggtgggaaaatagaccaataggcagagagagtcagtgcctatcagaaacccaagagtcttctctgtctccacatgcccagtttctattggtctccttaaacctgtcttgtaaccttgataccaaccttcccagggtttctcctccagcggcttccacattcaccttgtcccacaggcttgtgatagtagccttgtcctcctctgtgaaatgacccat

SEQ ID NO: 5 - DNA, 831 bp, Artificial Sequence
  Delta-beta hybrid
tcaatggtacttgtgagccagggcattagccacaccagccaccaccttctgataggcagcctgcatttgtggggtgaattccttgccaaagttgcgggccagcacacacaccagcacattgcccaagagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgagaaaaagtgcccttgaggttgtccaggtgagccaggccatcactaaaggcacctagcaccttcttgccatgagccttcaccttagggttgcccataacagcatcaggagaggacagatccccaaaggactcaaagaacctctgggtccaagggtagaccaccagtaatctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcactttgccccacagggcattgacagcagtcttctcctcaggagtcagatgcaccat

SEQ ID NO: 6 - DNA, 848 bp, Artificial Sequence
  Delta-beta hybrid AS1 (G16D)
tcaatggtacttgtgagccagggcattagccacaccagccaccaccttctgataggcagcctgcatttgtggggtgaattccttgccaaagttgcgggccagcacacacaccagcacattgcccaagagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcacgggtcccatagactcaccctgaagttctcaggatccacgtgcagcttgtcacagtgcagctcactcagctgagaaaaagtgcccttgaggttgtccaggtgagccaggccatcactaaaggcacctagcaccttcttgccatgagccttcaccttagggttgcccataacagcatcaggagaggacagatccccaaaggactcaaagaacctctgggtccaagggtagaccaccagtaatctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcactttgtcccacagggcattgacagcagtcttctcctcaggagtcagatgcaccat

SEQ ID NO: 7 - DNA, 831 bp, Artificial Sequence
  Beta AS1 (T87Q) (modified to avoid targeting by gRNA D)
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccagagagcggtcacagcggacttctcctcaggagtcaggtgcaccat

SEQ ID NO: 8 - DNA, 831 bp, Artificial Sequence
  Beta AS3 (modified to avoid targeting by gRNA D)
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccat

SEQ ID NO: 9 - DNA, 20 bp, Artificial Sequence
  Primer HBBex1 F
cagcatcaggagtggacaga

SEQ ID NO: 10 - DNA, 20 bp, Artificial Sequence
  Primer HBBex1 R
agtcagggcagagccatcta

SEQ ID NO: 11 - DNA, 22 bp, Artificial Sequence
  Primer HBB F
gcaaggtgaacgtggatgaagt

SEQ ID NO: 12 - DNA, 23 bp, Artificial Sequence
  Primer HBB R
taacagcatcaggagtggacaga

SEQ ID NO: 13 - DNA, 21 bp, Artificial Sequence
  Primer HBA1 F
cggtcaacttcaagctcctaa

SEQ ID NO: 14 - DNA, 20 bp, Artificial Sequence
  Primer HBA1 R
acagaagccaggaacttgtc

SEQ ID NO: 15 - DNA, 24 bp, Artificial Sequence
  Oligo FOR-Opt_gRNA B
caccgtaacggcagacttctcctc

SEQ ID NO: 16 - DNA, 24 bp, Artificial Sequence
  Oligo REV-Opt_gRNA B
aaacgaggagaagtctgccgttac

SEQ ID NO: 17 - DNA, 24 bp, Artificial Sequence
  Oligo FOR-Opt_gRNA D
caccgtctgccgttactgccctgt

SEQ ID NO: 18 - DNA, 24 bp, Artificial Sequence
  Oligo REV-Opt_gRNA D
aaacacagggcagtaacggcagac

SEQ ID NO: 19 - DNA, 25 bp, Artificial Sequence
  Oligo FOR-Opt_gRNA E
caccgaaggtgaacgtggatgaagt

SEQ ID NO: 20 - DNA, 25 bp, Artificial Sequence
  Oligo REV-Opt_gRNA E
aaacacttcatccacgttcaccttc

SEQ ID NO: 21 - DNA, 18 bp, Artificial Sequence
  Primer HBB-AS3 F
aagggcacctttgcccag

SEQ ID NO: 22 - DNA, 22 bp, Artificial Sequence
  Primer HBB-AS3 R
gccaccactttctgataggcag

SEQ ID NO: 23 - DNA, 20 bp, Artificial Sequence
  gRNA A spacer
cttgccccacagggcagtaa

SEQ ID NO: 24 - DNA, 20 bp, Artificial Sequence
  gRNA B spacer
gtaacggcagacttctcctc

SEQ ID NO: 25 - DNA, 19 bp, Artificial Sequence
  gRNA D spacer
tctgccgttactgccctgt

SEQ ID NO: 26 - DNA, 20 bp, Artificial Sequence
  gRNA E spacer
aaggtgaacgtggatgaagt

SEQ ID NO: 27 - DNA, 20 bp, Artificial Sequence
  gRNA F spacer
gtgtggcaaaggtgcccttg

SEQ ID NO: 28 - DNA, 20 bp, Artificial Sequence
  gRNA G spacer
acagtgcagctcactcagtg

SEQ ID NO: 29 - DNA, 20 bp, Artificial Sequence
  gRNA H spacer
tctgccgttactgccctgtg

SEQ ID NO: 30 - DNA, 20 bp, Artificial Sequence
  gRNA I spacer
cgttactgccctgtggggca

SEQ ID NO: 31 - DNA, 20 bp, Artificial Sequence
  gRNA J spacer
gtctgccgttactgccctgt

SEQ ID NO: 32 - DNA, 20 bp, Artificial Sequence
  gRNA K spacer
cagctcactcagtgtggcaa

SEQ ID NO: 33 - DNA, 20 bp, Artificial Sequence
  gRNA L spacer
tcccacccttaggctgctgg

SEQ ID NO: 34 - DNA, 20 bp, Artificial Sequence
  gRNA M spacer
ggctgctggtggtctaccct

SEQ ID NO: 35 - DNA, 20 bp, Artificial Sequence
  gRNA N spacer
ccccacagggcagtaacggc

SEQ ID NO: 36 - DNA, 20 bp, Artificial Sequence
  gRNA O spacer
taacggcagacttctccaca

SEQ ID NO: 37 - DNA, 27 bp, Artificial Sequence
  Oligo FOR-gRNA A
acaccgcttgccccacagggcagtaag

SEQ ID NO: 38 - DNA, 27 bp, Artificial Sequence
  Oligo REV-gRNA A
aaaacttactgccctgtggggcaagcg

SEQ ID NO: 39 - DNA, 26 bp, Artificial Sequence
  Oligo FOR-gRNA B
acaccgtaacggcagacttctcctcg

SEQ ID NO: 40 - DNA, 26 bp, Artificial Sequence
  Oligo REV-gRNA B
aaaacgaggagaagtctgccgttacg

SEQ ID NO: 41 - DNA, 26 bp, Artificial Sequence
  Oligo FOR-gRNA D
acaccgtctgccgttactgccctgtg

SEQ ID NO: 42 - DNA, 26 bp, Artificial Sequence
  Oligo REV-gRNA D
aaaacacagggcagtaacggcagacg

SEQ ID NO: 43 - DNA, 27 bp, Artificial Sequence
  Oligo FOR-gRNA E
acaccgaaggtgaacgtggatgaagtg

SEQ ID NO: 44 - DNA, 27 bp, Artificial Sequence
  Oligo REV-gRNA E
aaaacacttcatccacgttcaccttcg

SEQ ID NO: 45 - DNA, 10195 bp, Artificial Sequence
  LV.GLOBE.betaAS3-GLOBIN
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 46 - DNA, 10195 bp, Artificial Sequence
  LV.GLOBE.betaAS3-GLOBIN Sal1 (with restriction site for Sal1)
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccaaatggcggccgtacctttaagaccaatgacttacaaggcagctgtcgaccttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 47 - DNA, 10578 bp, Artificial Sequence
  LV.GLOBE.betaAS3-GLOBIN.gRNAD-OPTIMIZED (harboring the expression cassette for grna D - dang scaffold - U6 promoter)
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccaaatggcggccgtacctttaagaccaatgacttacaaggcagctgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctgccgttactgccctgtggtttcagagctatgctggaaacagcatagcaagttgaaataaggctagtccgttatcaacttgaaaaagtggcaccgagtcggtgctttttttgtcgaccttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 48 - DNA, 18 bp, Artificial Sequence
  gRNA against gamma-delta intergenic region
ggtgctctatacttccta

SEQ ID NO: 49 - DNA, 20 bp, Artificial Sequence
  gRNA A spacer inverted sequence
gaacggggtgtcccgtcatt

SEQ ID NO: 50 - DNA, 20 bp, Artificial Sequence
  HBG genes inverted sequence OFF-TARGET gRNA A
gaacggggtgtccgaacact

SEQ ID NO: 51 - DNA, 20 bp, Artificial Sequence
  HBD gene inverted sequence OFF-TARGET gRNA A
aaacggggtgtcccgtaact

SEQ ID NO: 52 - DNA, 20 bp, Artificial Sequence
  gRNA B spacer inverted sequence
cattgccgtctgaagaggag

SEQ ID NO: 53 - DNA, 20 bp, Artificial Sequence
  HBG genes inverted sequence OFF-TARGET gRNA B
tatcatcggaacaggaggag

SEQ ID NO: 54 - DNA, 20 bp, Artificial Sequence
  HBD gene inverted sequence OFF-TARGET gRNA B
aactgtcgtcagaagaggag

SEQ ID NO: 55 - DNA, 19 bp, Artificial Sequence
  gRNA D spacer inverted sequence
agacggcaatgacgggaca

SEQ ID NO: 56 - DNA, 19 bp, Artificial Sequence
  HBG genes inverted sequence OFF-TARGET gRNA D
cgatgatagtgttcggaca

SEQ ID NO: 57 - DNA, 19 bp, Artificial Sequence
  HBD gene inverted sequence OFF-TARGET gRNA D
tgacgacagttacgggaca

SEQ ID NO: 58 - DNA, 20 bp, Artificial Sequence
  gRNA E spacer inverted sequence
ttccacttgcacctacttca

SEQ ID NO: 59 - DNA, 20 bp, Artificial Sequence
  HBG genes inverted sequence OFF-TARGET gRNA E
ttccacttacaccttctacg

SEQ ID NO: 60 - DNA, 20 bp, Artificial Sequence
  HBD gene inverted sequence OFF-TARGET gRNA E
tttcacttgcacctacgtca

SEQ ID NO: 61 - RNA, 101 nt, Artificial Sequence
  ORIGINAL gRNA SCAFFOLD sequence
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuu

SEQ ID NO: 62 - RNA, 112 nt, Artificial Sequence
  OPTIMIZED gRNA SCAFFOLD sequence
  misc_feature (1)..(20): n is a, c, g, or u
nnnnnnnnnnnnnnnnnnnnguuucagagcuaugcuggaaacagcauagcaaguugaaauaaggcuaguccguuaucaacuugaaaaaguggcaccgagucggugcuuuuuu

SEQ ID NO: 63 - RNA, 101 nt, Artificial Sequence
  ORIGINAL SCAFFOLD gRNA B sequence
guaacggcagacuucuccucguuuuagagcuagaaauagcaaguuaaaauaaggcuaguccguuaucaacuugaaaaaguggcaccgagucggugcuuuuu

SEQ ID NO: 64 - RNA, 100 nt, Artificial Sequence
  ORIGINAL SCAFFOLD gRNA D sequence
ucugccguuacugcccuguguuuuagagcuagaaauagcaaguuaaaauaaggcuaguccguuaucaacuugaaaaaguggcaccgagucggugcuuuuu

SEQ ID NO: 65 - RNA, 101 nt, Artificial Sequence
  ORIGINAL SCAFFOLD gRNA E sequence
aaggugaacguggaugaaguguuuuagagcuagaaauagcaaguuaaaauaaggcuaguccguuaucaacuugaaaaaguggcaccgagucggugcuuuuu

SEQ ID NO: 66 - RNA, 113 nt, Artificial Sequence
  OPTIMIZED SCAFFOLD gRNA B sequence
guaacggcagacuucuccucgguuucagagcuaugcuggaaacagcauagcaaguugaaauaaggcuaguccguuaucaacuugaaaaaguggcaccgagucggugcuuuuuu

SEQ ID NO: 67 - RNA, 112 nt, Artificial Sequence
  OPTIMIZED SCAFFOLD gRNA D sequence
ucugccguuacugcccugugguuucagagcuaugcuggaaacagcauagcaaguugaaauaaggcuaguccguuaucaacuugaaaaaguggcaccgagucggugcuuuuuu

SEQ ID NO: 68 - RNA, 114 nt, Artificial Sequence
  OPTIMIZED SCAFFOLD gRNA E sequence
gaaggugaacguggaugaagugguuucagagcuaugcuggaaacagcauagcaaguugaaauaaggcuaguccguuaucaacuugaaaaaguggcaccgagucggugcuuuuuu

SEQ ID NO: 69 - DNA, 34 bp, Artificial Sequence
  HBB gene SCD sequence
cctgtggagaagtctgccgttactgccctgtggg

SEQ ID NO: 70 - protein, 11 aa, Artificial Sequence
  Beta-S globin protein sequence
PVEKSAVTALW

SEQ ID NO: 71 - DNA, 34 bp, Artificial Sequence
  HBB-AS3 modified sequence
cctgaggagaagtccgctgtgaccgctctctggg

SEQ ID NO: 72 - protein, 11 aa, Artificial Sequence
  Beta-AS3 globin sequence
PEEKSAVTALW

SEQ ID NO: 73 - DNA, 20 bp, Artificial Sequence
  gRNA 13bp-del spacer
cttgtcaaggctattggtca

SEQ ID NO: 74 - DNA, 20 bp, Artificial Sequence
  gRNA BCL11A spacer
cacaggctccaggaagggtt

SEQ ID NO: 75 - DNA, 10584 bp, Artificial Sequence
  LV.GLOBE-AS3modified.gRNA-BCL11Aenhancer
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcacaggctccaggaagggttgtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 76 - DNA, 10584 bp, Artificial Sequence
  LV.GLOBE-AS3modified.gRNA-13bp-del
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttgtcaaggctattggtcagtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 77 - DNA, 20 bp, Artificial Sequence
  BCL11A-TIDE FORWARD
tggacagcccgacagatgaa

SEQ ID NO: 78 - DNA, 20 bp, Artificial Sequence
  BCL11A-TIDE REVERSE
aaaagcgatacagggctggc

SEQ ID NO: 79 - DNA, 30 bp, Artificial Sequence
  13bp-del-TIDE FORWARD
aaaaacggctgacaaaagaagtcctggtat

SEQ ID NO: 80 - DNA, 30 bp, Artificial Sequence
  13bp-del-TIDE REVERSE
ataacctcagacgttccagaagcgagtgtg

SEQ ID NO: 81 - DNA, 19 bp, Artificial Sequence
  HBG1 + HBG2   FORWARD
cctgtcctctgcctctgcc

SEQ ID NO: 82 - DNA, 19 bp, Artificial Sequence
  HBG1 + HBG2 REVERSE
ggattgccaaaacggtcac

SEQ ID NO: 83 - DNA, 18 bp, Artificial Sequence
  HBB FORWARD
aagggcacctttgccaca

SEQ ID NO: 84 - DNA, 22 bp, Artificial Sequence
  HBB REVERSE
gccaccactttctgataggcag

SEQ ID NO: 85 - DNA, 19 bp, Artificial Sequence
  HBD FORWARD
caagggcactttttctcag

SEQ ID NO: 86 - DNA, 19 bp, Artificial Sequence
  HBD REVERSE
aattccttgccaaagttgc

SEQ ID NO: 87 - DNA, 20 bp, Artificial Sequence
  BCL11A FORWARD
aaccccagcacttaagcaaa

SEQ ID NO: 88 - DNA, 20 bp, Artificial Sequence
  BCL11A REVERSE
ggaggtcatgatccccttct

SEQ ID NO: 89 - DNA, 20 bp, Artificial Sequence
  BCL11AXL FORWARD
atgcgagctgtgcaactatg

SEQ ID NO: 90 - DNA, 20 bp, Artificial Sequence
  BCL11AXL REVERSE
gtaaacgtccttccccacct

SEQ ID NO: 91 - DNA, 26 bp, Artificial Sequence
  GAPDH FORWARD
cttcattgacctcaactacatggttt

SEQ ID NO: 92 - DNA, 22 bp, Artificial Sequence
  GAPDH REVERSE
tgggatttccattgatgacaag

SEQ ID NO: 93 - DNA, 10584 bp, Artificial Sequence
  LV.GLOBE-AS3modified.gRNA-luciferase
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttcgaaatgtccgttcggtgtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 94 - DNA, 10583 bp, Artificial Sequence
  LV.GLOBE-AS3modified.gRNAD
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctgccgttactgccctgtgtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg